SEQUENCE LISTING (80 sequences)

SEQ ID NO: 1 - DNA, 954 bp, Rattus sp. F12
atggaatcagggaacagcacaagaagattttcaagtttttttcttcttggatttacagaaaacccacaacttcacttcctcatttttgcactattcctgtccatgtacctggtaacagtgcttgggaacctgcttatcattatggccatcatcacacagtctcatttgcatacacccatgtactttttccttgctaacctatcctttgtggacatctgtttcacctccaccaccatcccaaagatgttggtaaatatatacacccagagcaagagcatcacctatgaagactgtattagccagatgtgtgtcttcttggttttcgcagaattgggcaactttctcctggctgtgatggcctatgaccgatatgtggctaactgtcacccactgtgttacacagtcattgtgaaccaccggctctgtatcctgctgcttctgctgtcctgggttatcagcattttccatgccttcatacagagcttaattgtgctacagttgaccttctgtggagatgtgaaaatccctcacttcttctgtgaacttaatcagctgtcccaactcacctgttcagacaactttccaagtcacctcataatgaatcttgtacctgttatgttggcagccatttccttcagtggcatcctttactcttatttcaagatagtatcctccatacattctatctccacagttcaggggaagtacaaggcattttctacttgtgcctctcacctttccattgtctccttattttatagtacaggcctcggagtgtacgtcagttctgctgtggtccaaagctcacattctgctgcaagtgcttcggtcatgtatactgtggtcacccccatgctgaaccccttcatttatagtctaaggaataaagatgtgaagagagctctggaaagactgttagaaggaaactgtaaagtgcatcattggactggatga

SEQ ID NO: 2 - DNA, 1002 bp, Rattus sp. F3
atggactcaagcaacaggacaagagtttcagaatttcttcttcttggatttgtagaaaacaaagacctacaaccccttatttatggtctttttctctctatgtacctggttactgtcattggaaacatatccattattgtggctatcatttcagatccctgtctgcacacccccatgtatttcttcctctctaacctgtcctttgtggacatctgtttcatttcaaccactgttccaaagatgttagtgaacatccagacccaaaacaatgtcatcacctatgcaggatgcattacccagatatactttttcttgctctttgtagaattggacaacttcttgctgactatcatggcctatgaccgttacgtagccatctgtcaccccatgcactacacagttatcatgaactacaagctctgtggatttctggttctggtatcttggattgtaagtgttctgcatgccttgtttcaaagcttgatgatgttggcgctgcccttctgcacacatctggaaatcccacactacttctgtgaacctaatcaggtgattcaactcacctgttctgatgcatttcttaatgatcttgtgatatattttacacttgtgctgctggctactgttcctcttgctggcatcttctattcttacttcaagatagtgtcctccatatgtgctatatcgtcagttcatgggaagtacaaagcattctccacctgtgcatctcacctttcagtcgtgtctttattttactgcacaggactaggagtgtacctcagttctgctgcaaacaacagctcacaggcaagtgccacagcctcagtcatgtacactgtagttacccctatggtgaacccttttatctatagtcttaggaataaagatgttaagagtgttctgaaaaaaactctttgtgaggaagttataaggagtccaccttccctacttcatttcttcctagtgttatgtcatctcccttgttttattttttgttattaa

SEQ ID NO: 3 - DNA, 942 bp, Rattus sp. F5
atgagcagcaccaaccagtccagtgtcaccgagttcctcctcctgggactctccaggcagccccagcagcagcagctcctcttcctgctcttcctcatcatgtacctggccactgtcctgggaaacctgctcatcatcctggctattggcacagactcccgcctgcacacccccatgtacttcttcctcagtaacctgtcctttgtggatgtctgcttctcctctaccactgtccctaaagttctggccaaccatatacttgggagtcaggccatttccttctctgggtgtctcacccagctgtattttctcgctgtgtttggtaacatggacaatttcctgctggctgtgatgtcctatgaccgatttgtggccatatgccaccctttacactacacaacaaagatgacccgtcagctctgtgtcctgcttgttgtggggtcatgggttgtagccaacatgaattgtctgttgcacatactgctcatggctcgactctccttctgtgcagacaacatgatcccccacttcttctgtgatggaactcccctcctgaaactctcctgctcagacacacatctcaatgagctgatgattcttacagagggagctgtggtcatggtcaccccatttgtctgcatcctcatctcctacatccacatcacctgtgctgtcctcagagtctcatcccccaggggaggatggaaatccttctccacctgtggctcccacctggctgtggtctgcctcttctatggcaccgtcatcgctgtgtatttcaacccatcatcctctcacttagctgggagggacatggcagctgcagtgatgtatgcagtggtgaccccaatgctgaaccctttcatctatagcctgaggaacagcgacatgaaagcagctttaaggaaagtgctcgccatgagatttccatctaagcagtaa

SEQ ID NO: 4 - DNA, 936 bp, Rattus sp. F6
atggcttggagtactggccagaacctgtccacaccaggaccattcatcttgctgggcttcccagggccaaggagcatgcgcattgggctcttcctgcttttcctggtcatgtatctgcttacggtagttggaaacctagccatcatctccctggtaggtgcccacagatgcctacagacacccatgtacttcttcctctgcaacctctccttcctggagatctggttcaccacagcctgcgtacccaagaccctggccacatttgcgcctcggggtggagtcatttccttggctggctgtgccacacagatgtactttgtcttttctttgggctgtaccgagtacttcctgctggctgtgatggcttatgaccgctacctggccatctgcctgccactgcgctatggtggcatcatgactcctgggctggcgatgcggttggccctgggatcctggctgtgtgggttttctgcaatcacagttcctgctaccctcattgcccgcctctctttctgtggctcacgtgtcatcaaccacttcttctgtgacatttcgccctggatagtgctttcctgcaccgacacgcaggtggtggaactggtgtcctttggcattgccttctgtgttattctgggctcgtgtggtatcacactagtctcctatgcttacatcatcactaccatcatcaagattccctctgcccggggccggcaccgcgccttctcaacctgctcatcccatctcactgtggtgctgatttggtatggctccaccatcttcttgcatgtgaggacctcggtagagagctccttggacctcaccaaagctatcacagtgctcaacaccattgtcacacctgtgctgaaccctttcatatatactctgaggaacaaggatgtcaaggaagctctgcgcaggacggtgaaggggaagtga

SEQ ID NO: 5 - DNA, 939 bp, Rattus sp. I14
atgactggaaataaccaaactttgatcttggagttcctcctcctgggtctgcccatcccatcagagtatcatctcctgttctatgccctgttcctggccatgtacctcaccatcatcctgggaaacctgctaatcattgtccttgttcgactggactctcatctccacatgcccatgtacttgtttctcagcaacttgtccttctctgacctctgcttttcctctgtcacaatgcccaaattgcttcagaacatgcagagccaagtaccatctatatcctatacaggctgcctgacacagctgtacttctttatggtttttggagatatggagagcttccttcttgtggtcatggcctatgaccgctatgtggccatttgctttcctttgcgttacaccaccatcatgagcaccaagttctgtgcttcactagtgctacttctgtggatgctgacgatgacccatgccctgctgcataccctactcattgctagattgtctttttgtgagaagaatgtgattcttcactttttctgtgacatttctgctcttctgaagttgtcctgctcagacatttatgttaatgagctgatgatatatatcttgggtggactcatcattattatcccattcctattaattgttatgtcctatgttagaattttcttctccattttgaagtttccatctattcaggacatctacaaggtattctcaacctgtggttcccatctgtctgtggtgaccttgttttatgggacaatttttggtatctacttatgtccatcaggtaataattctactgtgaaggagattgccatggctatgatgtacacagtggtgactcccatgctgaatcccttcatctacagcctgaggaacagagacatgaaaagggccctaataagagttatctgcactaagaaaatctctctgtaa

SEQ ID NO: 6 - DNA, 945 bp, Rattus sp. I15
atgacagaagagaaccaaactgtgatctcccagttccttctccttttcctgcccatcccctcagagcaccagcacgtgttctacgccctgttcctgtccatgtacctcaccactgtcctggggaacctcatcatcatcatcctcattcacctggactcccatctccacacacccatgtacttgtttctcagcaacttgtccttctctgatctctgcttttcctctgttacgatgcccaagttgttgcagaacatgcagagccaagttccatccatcccctttgcaggctgcctgacacaattatacttttacctgtattttgcagaccttgagagcttcctgcttgtggccatggcctatgaccgctatgtggccatctgcttcccccttcattacatgagcatcatgagccccaagctctgtgtgagtctggtggtgctgtcctgggtgctgaccaccttccatgccatgctgcacaccctgctcatggccagattgtcattctgtgcggacaatatgatcccccactttttctgtgatatatctcctttattgaaactgtcctgctctgacacgcatgttaatgagttggtgatatttgtcatgggagggcttgttattgtcattccatttgtgctcatcattgtatcttatgcacgagttgtcgcctccattcttaaagtcccttctgtccgaggcatccacaagatcttctccacctgcggctcccatctgtctgtggtgtcactgttctatgggacaatcattggtctctacttatgtccgtcagctaataactctactgtgaaggagactgtcatggccatgatgtacacagtggtgacccccatgctgaaccccttcatctacagcctgaggaacagagacatgaaagaggcactgataagagtcctttgtaaaaagaaaattaccttctgtctatga

SEQ ID NO: 7 - DNA, 933 bp, Rattus sp. I3
atgaacaatcaaactttcatcacccaattccttctcctgggactgcccatccctgaagaacatcagcacctgttctatgccttgttcctggtcatgtacctcaccaccatcttgggaaacttgctaatcattgtacttgttcaactggactcccagctccacacacctatgtatttgtttctcagcaatttgtctttctctgatctatgtttttcctctgtcacaatgcccaagctgctgcagaacatgaggagccaggacacatccattccctatggaggctgcctggcacaaacatacttctttatggtttttggagatatggagagtttccttcttgtggccatggcctatgaccgctatgtggccatctgcttccctctgcattacaccagcatcatgagccccaagctctgtacttgtctagtgctgttattgtggatgctgacgacatcccatgccatgatgcacacactgcttgcagcaagattgtctttttgtgagaacaatgtggtcctcaacttcttctgtgacctatttgttctcctaaagctggcctgctcagacacttatattaatgagttgatgatatttatcatgagtacactcctcattattattccattcttcctcattgttatgtcctatgcaaggatcatatcctctattcttaaggttccatctacccaaggcatctgcaaggtcttctctacctgtggttcccatctgtctgtagtatcactgttctatgggacaattattggtctctacttatgtccagcaggtaataattccactgtaaaagagatggtcatggccatgatgtacactgtggtgacccccatgctgaatcccttcatctacagcctaaggaatagagatatgaagagggccctaataagagttatctgtagtatgaaaatcactctgtaa

SEQ ID NO: 8 - DNA, 984 bp, Rattus sp. I7
atggagcgaaggaaccacagtgggagagtgagtgaatttgtgttgctgggtttcccagctcctgccccactgcgagtactactatttttcctttctcttctggactatgtgttggtgttgactgaaaacatgctcatcattatagcaattaggaaccacccaaccctccacaaacccatgtattttttcttggctaatatgtcatttctggagatttggtatgtcactgttacgattcctaagatgctcgctggcttcattggttccaaggagaaccatggacagctgatctcctttgaggcatgcatgacacaactctactttttcctgggcttgggttgcacagagtgtgtccttcttgctgtgatggcctatgaccgctatgtggctatctgtcatccactccactaccccgtcattgtcagtagccggctatgtgtgcagatggcagctggatcctgggctggaggttttggtatctccatggttaaagttttccttatttctcgcctgtcttactgtggccccaacaccatcaaccactttttctgtgatgtgtctccattgctcaacctgtcatgcactgacatgtccacagcagagcttacagactttgtcctggccatttttattctgctgggaccgctctctgtcactggggcatcctacatggccatcacaggtgctgtgatgcgcatcccctcagctgctggccgccataaagccttttcaacctgtgcctcccacctcactgttgtgatcatcttctatgcagccagtattttcatctatgccaggcctaaggcactctcagcttttgacaccaacaagctggtctctgtactctacgctgtcattgtaccgttgttcaatcccatcatctactgcttgcgcaaccaagatgtcaaaagagcgctacgtcgcacgctgcacctggcccaggaccaggaggccaataccaacaaaggcagcaaaattggttag

SEQ ID NO: 9 - DNA, 939 bp, Rattus sp. I8
atgaacaacaaaactgtcatcacccatttcctcctcctgggattgcccatccccccagagcaccagcaactgttctttgccctgttcctgatcatgtacctcaccacctttctgggaaacctgctaattgttgtccttgttcaactggactctcatctccacacacccatgtacttgtttctcagcaacttgtccttctctgatctctgcttttcctctgttacaatgctgaaattgctgcaaaatatacagagccaagtaccatctatatcctatgcaggatgcctgacacagatattcttctttttgttgtttggctaccttgggaatttccttcttgtagccatggcctatgaccgctatgtggccatctgcttccctctgcattataccaacatcatgagccataagctctgtacttgtctcctgctggtattttggataatgacatcatctcatgccatgatgcacaccctgcttgcagcaagattgtctttttgtgagaacaatgtactcctcaactttttctgtgacctgtttgttctcctaaagttggcctgctcagacacttatgttaatgagttgatgatacatatcatgggcgtgatcatcattgttattccattcgtgctcattgttatatcctatgccaagatcatctcctccattcttaaggttccatctactcaaagcattcacaaggtcttctccacttgtggttctcatctctctgtggtgtctctgttctacgggacaattattggtctctatttatgtccatcaggtgataattttagtctaaaggggtctgccatggctatgatgtacacagtggtaactccaatgctgaacccgttcatctacagcctaagaaacagagacatgaagcaggccctaataagagttacctgtagcaagaaaatctctctgccatggtag

SEQ ID NO: 10 - DNA, 945 bp, Rattus sp. I9
atgactagaagaaaccaaactgccatctctcagttcttccttctgggcctgccattccccccagagtaccaacacctgttctatgccctgttcctggccatgtacctcaccactctcctggggaacctcatcatcatcatcctcattctactggactcccatctccacacacccatgtacttgtttctcagcaatttatcctttgccgacctctgtttttcctctgtcacaatgcccaagttgttgcagaacatgcagagccaagttccatccatcccctatgcagggtgcctggcacagatatacttctttctgttttttggagaccttggaaacttcctgcttgtggccatggcctatgaccgctatgtggccatctgcttcccccttcattacatgagcatcatgagccccaagctctgtgtgagtctggtggtgctgtcctgggtgctgactaccttccatgccatgctgcacaccctgctcatggccagattgtcattctgtgaggacagtgtgatccctcactatttctgtgatatgtctactctgctgaaagtggcttgttctgacacccatgataatgaattagcaatatttatcttagggggccctatagttgtactacctttccttctcatcattgtttcttatgcaagaattgtttcctccatcttcaaggtcccttcttctcaaagcatccataaagccttctccacctgtggctcccacctgtctgtggtgtcactgttctatgggacagtcattggtctctacttatgtccttcagctaataactccactgtgaaggagactgtcatgtctttgatgtacacaatggtgacacccatgctgaaccccttcatctacagcctaagaaacagagacataaaagatgcattagaaaaaataatgtgcaaaaagcaaattccctcctttctatga

SEQ ID NO: 11 - DNA, 645 bp, Homo Sapiens H5
  misc_feature ()..(): n = unknown
atctgttttgtgtctaccactgtcccaaagcagctggtgaacatccagacacagagcagagtcatcacctatgcagactgcatcacccagatgtgcttttttatactctttgtagtgttggacagcttactcctgactgtgatggcctatgaccggtttgtggccatctgtcaccccctgcactacacagtcattatgagctcctggctctgtggactgctggttctggtgtcctggatcgtgagcatcctatattctctgttacaaagcataatggcattgcagctgtccttctgtacagaactgaaaatccctcaatttttctgtgaacttaatcaggtcatccaccttgcctgttccgacacttttattaatgacatgatgatgaattttacaagtgtgctgctgggtgggggatgcctcgctggaatattttactnntactttaagatactttgttgcatatgttcgatctcatcagctcaggggatgaataaagcactttccacctgtgcatctcacctctcagttgtctccttattttattgtacaggcgtaggtgtgtaccttagttctgctgcaacccataactcactctcaaatgctgcagcctcggtgatgtacactgtggtcacctccatgctg

SEQ ID NO: 12 - protein, 215 aa, Homo Sapiens H5
  UNSURE (147)..(147): x = unknown
ICFVSTTVPKQLVNIQTQSRVITYADCITQMCFFILFVVLDSLLLTVMAYDRFVAICHPLHYTVIMSSWLCGLLVLVSWIVSILYSLLQSIMALQLSFCTELKIPQFFCELNQVIHLACSDTFINDMMMNFTSVLLGGGCLAGIFYXYFKILCCICSISSAQGMNKALSTCASHLSVVSLFYCTGVGVYLSSAATHNSLSNAAASVMYTVVTSML

SEQ ID NO: 13 - DNA, 640 bp, Rattus sp. J1
  misc_feature ()..(): n = unknown
catctgctttacttctgctagcatcccaaagatgctagtgaatatacagacgaagaacaaggtgatcacctatgaaggctgcatctcccaagtatacttttcatactctttggagttttggacaactttcttctcgactgtgatggcctatgaccgatatgtggccatctgtcacccatctnactacacaggtcatcatgaaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttattcttactctaagatagtttcctccatacgagaaatctcatcatcacagggaaagtacaagnnattctccacctgtgcatcccacctctcagttgtttcattattctattctacacttttgggtgtgtaccttagttcttcttttacccaaaactcacactcaactgcacgggcatctgttatgtacagtgtggtcacccccatgttg

SEQ ID NO: 14 - protein, 213 aa, Rattus sp. J1
  UNSURE (61)..(165): x = unknown
ICFTSASIPKMLVNIQTKNKVITYEGCISQVYFSYSLEFWTTFFSTVMAYDRYVAICHPSXYTGHHEXXXXXXXXXXXXXXXXXXXXXXXXXXXXXXXXXXXXXXXXXXXXXXXXXXXXXXXXXXXXXXXXXXXXXXXXXXXXYSYSKIVSSIREISSSQGKYKXFSTCASHLSVVSLFYSTLLGVYLSSSFTQNSHSTARASVMYSVVTPML

SEQ ID NO: 15 - DNA, 636 bp, Rattus sp. J2
acctccaccaccatcccaaagatgctggtaaatatacacacccagagcaatactatcacctatgaagactgtatttcccagatgtttgtactcttggtttttggagaactggacaactttctcctggctgtgatggcctatgatcgatatgtggctatctgtcacccactgtattacacagtcattgtgaaccaccgactctgtatcctgctgcttctgctgtcctgggttgtcagcattttacatgccttcttacagagcttaattgtactacagttgaccttctgtggagatgtgaaaatccctcacttcttctgtgagctcaatcagctgtcccaactcacatgttcagacaactttccaagtcacctcacaatgcatcttgtacctgttatatttgcagctatttccctcagtggtatcctttactcttatttcaagatagtgtcttccatacgttctatgtcctcagttcaagggaagtacaaggcattttctacatgtgcctctcacctttccattgtctccttattttatagtacaggcctcggggtgtacgtcagttctgctgtgatccgaagctcacactcctctgcaagtgcttcggtcatgtatactgtggtcacccccatgttg

SEQ ID NO: 16 - protein, 212 aa, Rattus sp. J2
TSTTIPKMLVNIHTQSNTITYEDCISQMFVLLVFGELDNFLLAVMAYDRYVAICHPLYYTVIVNHRLCILLLLLSWVVSILHAFLQSLIVLQLTFCGDVKIPHFFCELNQLSQLTCSDNFPSHLTMHLVPVIFAAISLSGILYSYFKIVSSIRSMSSVQGKYKAFSTCASHLSIVSLFYSTGLGVYVSSAVIRSSHSSASASVMYTVVTPML

SEQ ID NO: 17 - DNA, 646 bp, Rattus sp. J4
cataggctattcatcttctgtcacacccaatatgcttgtcaacttccttataaagcaaaataccatctcataccttggatgttctatacagtttggctcagctgctttgtttggaggtcttgaatgcttccttctggctgccatggcgtatgatcgttttgtagcaatctgcaacccactgctttattcaacgaaaatgtccacacaagtctgtgtccagttggttgtgggatcttatatagggggatttcttaatgcctcctcttttaccctttcctttttttccttgtccttctgtggaccaaatagaatcaatcacttttactgtgattttgctccgttagtagaactttcttgctctgatgtcagtgttcctgatgctgttacctcattttctgctgcctcagttactatgctcacagtgtttatcatagccatctcctatacctatatcctcatcaccatcctgaagatgcgttccactgagggtcgacagaaagcattctctacctgcacttcccacctcactgcagtcactctgtgctatggaaccatcacattcatctatgtgatgcccaagtccagctactccacagaccagaacaaggtggtgtctgtgttttatatggtggtgatccccatgttg

SEQ ID NO: 18 - protein, 215 aa, Rattus sp. J4
IGYSSSVTPNMLVNFLIKQNTISYLGCSIQFGSAALFGGLECFLLAAMAYDRFVAICNPLLYSTKMSTQVCVQLVVGSYIGGFLNASSFTLSFFSLSFCGPNRINHFYCDFAPLVELSCSDVSVPDAVTSFSAASVTMLTVFIIAISYTYILITILKMRSTEGRQKAFSTCTSHLTAVTLCYGTITFIYVMPKSSYSTDQNKVVSVFYMVVIPML

SEQ ID NO: 19 - DNA, 481 bp, Rattus sp. J7
catctgcaagcccctgcactacaccaccatcatgaataaccgagtgtgcacagttctagtcctctcctgttggtttgctggcctgttgatcatcctcccacctcttggtcatggcctccagctggagttctgtgactccaatgtgattgatcattttggctgtgatgcctctccaattctgcagataacctgctcagacacggtatttatagagaaaattgtcttggcttttgccatactgacactcatcattactctggtatgtgttgttctctcctacacatacatcatcaagaccattttaaagtttccttctgctcaacaaagaaaaaaggccttttctacatgttcttcccacatgattgtggtttccatcacctatgggagctgtattttcatctacatcaaaccttcagcgaaggaaggggtagccatcaataaggttgtatctgtgctcacaacatcagtcgcccctttgctc

SEQ ID NO: 20 - protein, 160 aa, Rattus sp. J7
ICKPLHYTTIMNNRVCTVLVLSCWFAGLLIILPPLGHGLQLEFCDSNVIDHFGCDASPILQITCSDTVFIEKIVLAFAILTLIITLVCVVLSYTYIIKTILKFPSAQQRKKAFSTCSSHMIVVSITYGSCIFIYIKPSAKEGVAINKVVSVLTTSVAPLL

SEQ ID NO: 21 - DNA, 481 bp, Rattus sp. J8
  misc_feature ()..(): n = unknown
catctgccacccgctccactactctcttctcatgagtcctgacaactgtgctgctctggtaacagtctcctgggtgacaggggtgggcacgggcttcctgccttccctcctgatttctaagttggacttctgtgggcccaaccgcatcaaccatttcttctgtgacctccctccattaatccagctgtcctgctccagcgtctttgtgacagaaatggccatctttgtcctgtccatcgctgtgctctgcatctgtttcctcctaacccnnnnntcctacattttcatagtgtcctccattctgagaatcccttccactaccggcaggatgaagacattttctacatgtggctcccacctggccgtggtcaccatctactatgggaccatgatctccatgtatgtcggcccaaatgcgcatctgtccccggagctcaacaaggtcatttctgtcttctacactgtgatcaccccactactg

SEQ ID NO: 22 - protein, 160 aa, Rattus sp. J8
  UNSURE (90)..(91): x = unknown
ICHPLHYSLLMSPDNCAALVTVSWVTGVGTGFLPSLLISKLDFCGPNRINHFFCDLPPLIQLSCSSVFVTEMAIFVLSIAVLCICFLLTXXSYIFIVSSILRIPSTTGRMKTFSTCGSHLAVVTIYYGTMISMYVGPNAHLSPELNKVISVFYTVITPLL

SEQ ID NO: 23 - DNA, 646 bp, Rattus sp. J11
  misc_feature ()..(): n = unknown
ngtctgcttctcctccaccactgtccccaaggtactggctaaccacatactcagtagtcaggccatttccttctctgggtgtctaactcagctgtattttctctgtgtgtctgtgaatatggacaatttcctgctggctgtgatggcctatgacagatttgtggccatatgccaccctttgtactacacaacaaagatgacccaccagctctgtgtcttgctggtgtctggatcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtgatcatggtcaccccatttgtctgcatcctcatctcttacatctacatcaccaatgcagtcctcagagtctcatcctttaggggaggatggaaagccttctccacctgtggctcacacctggctgtggtctgcctcttctatggcaccatcattgctgtgtatttcaatcctgtatcttcccattcatctgagaaggacactgcagcaactgtgctatacacagtggtgactcccatgttg

SEQ ID NO: 24 - protein, 215 aa, Rattus sp. J11
  UNSURE (79)..(134): x = unknown
VCFSSTTVPKVLANHILSSQAISFSGCLTQLYFLCVSVNMDNFLLAVMAYDRFVAICHPLYYTTKMTHQLCVLLVSGSXXXXXXXXXXXXXXXXXXXXXXXXXXXXXXXXXXXXXXXXXXXXXXXXXXXXXXXXVIMVTPFVCILISYIYITNAVLRVSSFRGGWKAFSTCGSHLAVVCLFYGTIIAVYFNPVSSHSSEKDTAATVLYTVVTPML

SEQ ID NO: 25 - DNA, 646 bp, Rattus sp. J14
  misc_feature ()..(): n = unknown
tgtctgcttctcctccaccactgtccccaaggtactggctaaccacatactcagtagtcaggccatttccttctctgggtgtctaactcagctgtattttctctgtgtgtctgtgaatatggacaatttcctgctggctgtgatggcctatgacagatttgtggccatatgccaccctttgtactacacaacaccgatgacccaccagctctgtgtcttgctggtgtctggatcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtgatcatggtcaccccatttgtctgcatcctcatctcttacatctacatcaccaatgcagtcctcagagtctcatcctttaggggaggatggaaagccttctccacctgtggctcacacctggctgtggtctgcctcttctatggcaccatcattgctgtgtatttcaatcctgtatcttcccattcatctgagaaggacactgcagcaactgtgctatacacagtggtgactcccatgttg

SEQ ID NO: 26 - protein, 215 aa, Rattus sp. J14
  UNSURE (79)..(134): x = unknown
VCFSSTTVPKVLANHILSSQAISFSGCLTQLYFLCVSVNMDNFLLAVMAYDRFVAICHPLYYTTPMTHQLCVLLVSGSXXXXXXXXXXXXXXXXXXXXXXXXXXXXXXXXXXXXXXXXXXXXXXXXXXXXXXXXVIMVTPFVCILISYIYITNAVLRVSSFRGGWKAFSTCGSHLAVVCLFYGTIIAVYFNPVSSHSSEKDTAATVLYTVVTPML

SEQ ID NO: 27 - DNA, 481 bp, Rattus sp. J15
  misc_feature ()..(): x = unknown
tatctgcaaccctctgcgctacccagtgctcatgagcggccgggtgtgcctgctcatggtcgtggcctcctggttgggaggatccctcaacgcctccattcagacttctctgacccttcagttcccctactgtggatcacggaagatctcccacttcttctgtgaggtgccctcgctgctgannntggcctgtgcagacactgaagcctatgagcaggtactatttgtgacaggcgtggtggtcctcctggtgcccattacattcattactgcctcttatgccctcatcctggctgctgtgctccgaatgcactctgcggaggggagtcagaaggccctagccacatgctcctctcacctgacagtcgtcaatctcttctatgggccccttgtctacacctacatgttacctgcttcctatcactcaccaggccaagacgacatagtatccgtcttttacaccgttctcacacccatgctt

SEQ ID NO: 28 - protein, 160 aa, Rattus sp. J15
  UNSURE (61)..(62): x = unknown
ICNPLRYPVLMSGRVCLLMVVASWLGGSLNASIQTSLTLQFPYCGSRKISHFFCEVPSLLXXACADTEAYEQVLFVTGVVVLLVPITFITASYALILAAVLRMHSAEGSQKALATCSSHLTVVNLFYGPLVYTYMLPASYHSPGQDDIVSVFYTVLTPML

SEQ ID NO: 29 - DNA, 481 bp, Rattus sp. J16
catctgtaggcctcttcactatcctaccctcatgacccagacactgtgtgccaagattgccactggttgctggttgggaggcttggctgggccagtggtagaaatttccttggtgtctcgtctccttttttgtggccccaatcacattcaacacatcttttgtgatttcccacctgtgctgagcttggcttgtactgatacatcagtgaatgtcctggtagattttattataaacctctgcaagatcctggccaccttcctgctgatcctgagctcctacttgcagataatccgcacagtgctcaagattccttcagctgcaggcaagaagaaagcattctcgacttgtgcctcccatctcactgtggttctcatcttctatgggagcatccttttcatgtatgtgcggctgaagaagacttactcccttgactacgacagagccttggcagtagtctactccgtggttacccctttcctg

SEQ ID NO: 30 - protein, 160 aa, Rattus sp. J16
ICRPLHYPTLMTQTLCAKIATGCWLGGLAGPVVEISLVSRLLFCGPNHIQHIFCDFPPVLSLACTDTSVNVLVDFIINLCKILATFLLILSSYLQIIRTVLKIPSAAGKKKAFSTCASHLTVVLIFYGSILFMYVRLKKTYSLDYDRALAVVYSVVTPFL

SEQ ID NO: 31 - DNA, 481 bp, Rattus sp. J17
  misc_feature ()..(): n =  unknown
aatctgcaacccactgctttattccaccaaaatgtccacacaagtctgtatccagttggttgcaggatcttatatagggggttttcttaatacttgcctcatcatgttttactttttctcttttctcttctgtgggccaaatatagttgatcattttttctgtgattttgctcctttnntggaactttcgtgctctgatgtgagtgtctctgtagttgttatgtcattttctgctggctcagttactatgatcacagtgtttatcatagccatctcctattcttacatcctcatcaccatcctgaagatgtcctcaactgagggccgtcacaaggctttctccacatgtacctcccacctcactgcagtcactctctactatggcaccattaccttcatttatgtgatgcccaagtccacatactctacagaccagaacaaggtggtgtctgtgttttacatggtggtgatcccaatgttg

SEQ ID NO: 32 - protein, 160 aa, Rattus sp. J17
  UNSURE (59)..(60): x = unknown
ICNPLLYSTKMSTQVCIQLVAGSYIGGFLNTCLIMFYFFSFLFCGPNIVDHFFCDFAPXXELSCSDVSVSVVVMSFSAGSVTMITVFIIAISYSYILITILKMSSTEGRHKAFSTCTSHLTAVTLYYGTITFIYVMPKSTYSTDQNKVVSVFYMVVIPML

SEQ ID NO: 33 - DNA, 479 bp, Rattus sp. J19
tatctgccaccctctgaagtacacagttatcatgaatcactatttttgtgtgatgctgctgctcttctctgtgttcgttagcattgcacatgcgttgttccacattttaatggtgttgatactgactttcagcacaaaaactgaaatccctcactttttctgtgagctggctcatatcatcaaacttacctgttccgataattttatcaactatctgctgatatacacagagtctgtcttattttttggtgttcatattgtagggatcattttgtcttatatttacactgtatcctcagttttaagaatgtcattattgggaggaatgtataaagccttttcaacatgtggatctcatttgtcggttgtctctgttttatggcacaggttttggggtacacataagctctccacttactgactctccaaggaagactgtagtggcttcagtgatgtacactgtggttactcagatgctg

SEQ ID NO: 34 - protein, 139 aa, Rattus sp. J19
ICHPLKYTVIMNHYFCVMLLLFSVFVSIAHALFHILMVLILTFSTKTEIPHFFCELAHIIKLTCSDNFINYLLIYTESVLFFGVHIVGIILSYIYTVSSVLRMSLLGGMYKAFSTCGSHLSVVSVLWHRFWGTHKLSTY

SEQ ID NO: 35 - DNA, 480 bp, Rattus sp. J20
  misc_feature ()..(): n = unknown
aatctgctacccactgaggtaccttctcatcatgagctgggtggtgtgcacagcactgtccgtggcaatctgggtcataggcttttgtgcctccgttatacctctctgcttcacgatcctcccactctgtggtccttacgtcgttgattatcttttctgcgagctgcccatccttctgcacctgttctgcacagatacatctctgctggagnnnnnnnnnnnnnnnnnnnnnnnnnnnnnnnnnnnnnnnnncccttcctcctgattgttctctcctaccttcgcatcctggtggctgtgataagaatagactcagctgagggcagaaaaaaggccttttcaacttgtgcttcacacttggctgtggtgaccatctactatggaacagggctgatcaggtacttgaggcccaagtccctttattccgctgagggagacagactgatctctgtgttctatgcagtcattggccctgcactg

SEQ ID NO: 36 - protein, 160 aa, Rattus sp. J2O
  UNSURE (71)..(84): x = unknown
ICYPLRYLLIMSWVVCTALSVAIWVIGFCASVIPLCFTILPLCGPYVVDYLFCELPILLHLFCTDTSLLEXXXXXXXXXXXXXXPFLLIVLSYLRILVAVIRIDSAEGRKKAFSTCASHLAVVTIYYGTGLIRYLRPKSLYSAEGDRLISVFYAVIGPAL

SEQ ID NO: 37 - DNA, 35 bp, artificial - primer A1
  modified_base (9)..(9): i
aantnnatnntnntnaanntngcngtngcngcnga

SEQ ID NO: 38 - DNA, 32 bp, artificial - primer A2
  misc_feature (3)..(3): n = c or t
aantanttnntnntnaanctngcnntngcnga

SEQ ID NO: 39 - DNA, 32 bp, artificial - primer A3
  misc_feature (3)..(4): n = c or t
aannnnttnntnatnncnctngcntnngcnga

SEQ ID NO: 40 - DNA, 32 bp, artificial - primer A4
  misc_feature (1)..(1): n = c or a
ngnttnntnatgtgnaanctnnnnttngcnga

SEQ ID NO: 41 - DNA, 32 bp, artificial - primer A5
  modified_base (3)..(3): i
acngtntanatnacncanntnnnnatngcnga

SEQ ID NO: 42 - DNA, 33 bp, artificial - primer B1
  modified_base (4)..(4): i
ctgnnnnttcatnannnnntanannannggntt

SEQ ID NO: 43 - DNA, 31 bp, artificial - primer B2
  misc_feature (1)..(1): n =  g or t
nntnnttnagncancantanatnatnggntt

SEQ ID NO: 44 - DNA, 32 bp, artificial - primer B3
  modified_base (3)..(3): i
tcnatnttnaangtngtntanatnatnggntt

SEQ ID NO: 45 - DNA, 32 bp, artificial - primer B4
  misc_feature (3)..(3): n = c or t
gcnttngtnaanatngcntanagnaanggntt

SEQ ID NO: 46 - DNA, 32 bp, artificial - primer B5
  misc_feature (3)..(3): n = a or g
aantcnggnnnncgnnantanatnannggntt

SEQ ID NO: 47 - DNA, 32 bp, artificial - primer B6
  misc_feature (1)..(1): n = g or c
nnnnnnccnacnaanaantanatnaanggntt

SEQ ID NO: 48 - DNA, 23 bp, artificial - primer P1
  modified_base (6)..(6): i
atggcntanganngntangtngc

SEQ ID NO: 49 - DNA, 29 bp, artificial - primer P4
  modified_base (3)..(3): i
aanannnnnacnannnnnanntgnnnnnc

SEQ ID NO: 50 - protein, 6 aa, artificial - motif
KIVSSI

SEQ ID NO: 51 - protein, 6 aa, artificial - motif
RIVSSI

SEQ ID NO: 52 - protein, 6 aa, artificial - motif
HITCAV

SEQ ID NO: 53 - protein, 6 aa, artificial - motif
HITWAV

SEQ ID NO: 54 - protein, 19 aa, Rattus sp.
LSKEDCSGFSDVHCGYSDA

SEQ ID NO: 55 - protein, 9 aa, Artificial - motif
  UNSURE (2)..(7): x = unknown
LXXPMYXFL

SEQ ID NO: 56 - protein, 9 aa, Artificial - motif
  VARIANT (2)..(2): X = H or Q
LXXPMYXFL

SEQ ID NO: 57 - protein, 10 aa, Artificial - motif
  UNSURE (2)..(7): X = UNKNOWN
MXYDRXXAIC

SEQ ID NO: 58 - protein, 10 aa, Artificial - motif
  VARIANT (2)..(2): X = A OR S
MXYDRXXAIC

SEQ ID NO: 59 - protein, 7 aa, Artificial - motif
  UNSURE (3)..(4): X = Unknown
DRXXAIC

SEQ ID NO: 60 - protein, 7 aa, Artificial - motif
  VARIANT (3)..(3): X = F or Y
DRXXAIC

SEQ ID NO: 61 - protein, 9 aa, Artificial - motif
  UNSURE (2)..(7): X = Unknown
XXFSTCXSH

SEQ ID NO: 62 - protein, 9 aa, Artificial - motif
  VARIANT (1)..(1): X = K or R
XXFSTCXSH

SEQ ID NO: 63 - protein, 7 aa, Artificial - motif
  UNSURE (5)..(5): X = Unknown
FSTCXSH

SEQ ID NO: 64 - protein, 7 aa, Artificial - motif
  VARIANT (5)..(5): X = A or G or S
FSTCXSH

SEQ ID NO: 65 - protein, 12 aa, Artificial - motif
  UNSURE (2)..(9): X = Unknown
PXXNPXIYXLRN

SEQ ID NO: 66 - protein, 12 aa, Artificial - motif
  VARIANT (2)..(2): X = M or L or V
PXXNPXIYXLRN

SEQ ID NO: 67 - protein, 8 aa, Artificial - motif
  UNSURE (2)..(6): X = Unknown
PXXNPXIY

SEQ ID NO: 68 - protein, 8 aa, Artificial - motif
  VARIANT (2)..(2): X = M or L or V
PXXNPXIY

SEQ ID NO: 69 - protein, 9 aa, Artificial - motif
  UNSURE (3)..(6): X = Unknown
NPXIYXLRN

SEQ ID NO: 70 - protein, 9 aa, Artificial - motif
  VARIANT (3)..(3): X = F or I
NPXIYXLRN

SEQ ID NO: 71 - protein, 333 aa, Rattus sp. F3
MDSSNRTRVSEFLLLGFVENKDLQPLIYGLFLSMYLVTVIGNISIIVAIISDPCLHTPMYFFLSNLSFVDICFISTTVPKMLVNIQTQNNVITYAGCITQIYFFLLFVELDNFLLTIMAYDRYVAICHPMHYTVIMNYKLCGFLVLVSWIVSVLHALFQSLMMLALPFCTHLEIPHYFCEPNQVIQLTCSDAFLNDLVIYFTLVLLATVPLAGIFYSYFKIVSSICAISSVHGKYKAFSTCASHLSVVSLFYCTGLGVYLSSAANNSSQASATASVMYTVVTPMVNPFIYSLRNKDVKSVLKKTLCEEVIRSPPSLLHFFLVLCHLPCFIFCY

SEQ ID NO: 72 - protein, 313 aa, Rattus sp. F5
MSSTNQSSVTEFLLLGLSRQPQQQQLLFLLFLIMYLATVLGNLLIILAIGTDSRLHTPMYFFLSNLSFVDVCFSSTTVPKVLANHILGSQAISFSGCLTQLYFLAVFGNMDNFLLAVMSYDRFVAICHPLHYTTKMTRQLCVLLVVGSWVVANMNCLLHILLMARLSFCADNMIPHFFCDGTPLLKLSCSDTHLNELMILTEGAVVMVTPFVCILISYIHITCAVLRVSSPRGGWKSFSTCGSHLAVVCLFYGTVIAVYFNPSSSHLAGRDMAAAVMYAVVTPMLNPFIYSLRNSDMKAALRKVLAMRFPSKQ

SEQ ID NO: 73 - protein, 311 aa, Rattus sp. F6
MAWSTGQNLSTPGPFILLGFPGPRSMRIGLFLLFLVMYLLTVVGNLAIISLVGAHRCLQTPMYFFLCNLSFLEIWFTTACVPKTLATFAPRGGVISLAGCATQMYFVFSLGCTEYFLLAVMAYDRYLAICLPLRYGGIMTPGLAMRLALGSWLCGFSAITVPATLIARLSFCGSRVINHFFCDISPWIVLSCTDTQVVELVSFGIAFCVILGSCGITLVSYAYIITTIIKIPSARGRHRAFSTCSSHLTVVLIWYGSTIFLHVRTSVESSLDLTKAITVLNTIVTPVLNPFIYTLRNKDVKEALRRTVKGK

SEQ ID NO: 74 - protein, 317 aa, Rattus sp. F12
MESGNSTRRFSSFFLLGFTENPQLHFLIFALFLSMYLVTVLGNLLIIMAIITQSHLHTPMYFFLANLSFVDICFTSTTIPKMLVNIYTQSKSITYEDCISQMCVFLVFAELGNFLLAVMAYDRYVANCHPLCYTVIVNHRLCILLLLLSWVISIFHAFIQSLIVLQLTFCGDVKIPHFFCELNQLSQLTCSDNFPSHLIMNLVPVMLAAISFSGILYSYFKIVSSIHSISTVQGKYKAFSTCASHLSIVSLFYSTGLGVYVSSAVVQSSHSAASASVMYTVVTPMLNPFIYSLRNKDVKRALERLLEGNCKVHHWTG

SEQ ID NO: 75 - protein, 310 aa, Rattus sp. I3
MNNQTFITQFLLLGLPIPEEHQHLFYALFLVMYLTTILGNLLIIVLVQLDSQLHTPMYLFLSNLSFSDLCFSSVTMPKLLQNMRSQDTSIPYGGCLAQTYFFMVFGDMESFLLVAMAYDRYVAICFPLHYTSIMSPKLCTCLVLLLWMLTTSHAMMHTLLAARLSFCENNVVLNFFCDLFVLLKLACSDTYINELMIFIMSTLLIIIPFFLIVMSYARIISSILKVPSTQGICKVFSTCGSHLSVVSLFYGTIIGLYLCPAGNNSTVKEMVMAMMYTVVTPMLNPFIYSLRNRDMKRALIRVICSMKITL

SEQ ID NO: 76 - protein, 327 aa, Rattus sp. I7
MERRNHSGRVSEFVLLGFPAPAPLRVLLFFLSLLDYVLVLTENMLIIIAIRNHPTLHKPMYFFLANMSFLEIWYVTVTIPKMLAGFIGSKENHGQLISFEACMTQLYFFLGLGCTECVLLAVMAYDRYVAICHPLHYPVIVSSRLCVQMAAGSWAGGFGISMVKVFLISRLSYCGPNTINHFFCDVSPLLNLSCTDMSTAELTDFVLAIFILLGPLSVTGASYMAITGAVMRIPSAAGRHKAFSTCASHLTVVIIFYAASIFIYARPKALSAFDTNKLVSVLYAVIVPLFNPIIYCLRNQDVKRALRRTLHLAQDQEANTNKGSKIG

SEQ ID NO: 77 - protein, 312 aa, Rattus sp. I8
MNNKTVITHFLLLGLPIPPEHQQLFFALFLIMYLTTFLGNLLIVVLVQLDSHLHTPMYLFLSNLSFSDLCFSSVTMLKLLQNIQSQVPSISYAGCLTQIFFFLLFGYLGNFLLVAMAYDRYVAICFPLHYTNIMSHKLCTCLLLVFWIMTSSHAMMHTLLAARLSFCENNVLLNFFCDLFVLLKLACSDTYVNELMIHIMGVIIIVIPFVLIVISYAKIISSILKVPSTQSIHKVFSTCGSHLSVVSLFYGTIIGLYLCPSGDNFSLKGSAMAMMYTVVTPMLNPFIYSLRNRDMKQALIRVTCSKKISLPW

SEQ ID NO: 78 - protein, 314 aa, Rattus sp. I9
MTRRNQTAISQFFLLGLPFPPEYQHLFYALFLAMYLTTLLGNLIIIILILLDSHLHTPMYLFLSNLSFADLCFSSVTMPKLLQNMQSQVPSIPYAGCLAQIYFFLFFGDLGNFLLVAMAYDRYVAICFPLHYMSIMSPKLCVSLVVLSWVLTTFHAMLHTLLMARLSFCEDSVIPHYFCDMSTLLKVACSDTHDNELAIFILGGPIVVLPFLLIIVSYARIVSSIFKVPSSQSIHKAFSTCGSHLSVVSLFYGTVIGLYLCPSANNSTVKETVMSLMYTMVTPMLNPFIYSLRNRDIKDALEKIMCKKQIPSFL

SEQ ID NO: 79 - protein, 312 aa, Rattus sp. I14
MTGNNQTLILEFLLLGLPIPSEYHLLFYALFLAMYLTIILGNLLIIVLVRLDSHLHMPMYLFLSNLSFSDLCFSSVTMPKLLQNMQSQVPSISYTGCLTQLYFFMVFGDMESFLLVVMAYDRYVAICFPLRYTTIMSTKFCASLVLLLWMLTMTHALLHTLLIARLSFCEKNVILHFFCDISALLKLSCSDIYVNELMIYILGGLIIIIPFLLIVMSYVRIFFSILKFPSIQDIYKVFSTCGSHLSVVTLFYGTIFGIYLCPSGNNSTVKEIAMAMMYTVVTPMLNPFIYSLRNRDMKRALIRVICTKKISL

SEQ ID NO: 80 - protein, 314 aa, Rattus sp. I15
MTEENQTVISQFLLLFLPIPSEHQHVFYALFLSMYLTTVLGNLIIIILIHLDSHLHTPMYLFLSNLSFSDLCFSSVTMPKLLQNMQSQVPSIPFAGCLTQLYFYLYFADLESFLLVAMAYDRYVAICFPLHYMSIMSPKLCVSLVVLSWVLTTFHAMLHTLLMARLSFCADNMIPHFFCDISPLLKLSCSDTHVNELVIFVMGGLVIVIPFVLIIVSYARVVASILKVPSVRGIHKIFSTCGSHLSVVSLFYGTIIGLYLCPSANNSTVKETVMAMMYTVVTPMLNPFIYSLRNRDMKEALIRVLCKKKITFCL

(19) United States
(12) Patent Application Publication
(10) Pub. No.: US 2002/0064817 A1
(43) Pub. Date: May 30, 2002

(54) Odorant receptors and uses thereof

(76) Inventors: Linda B. Buck, Boston, MA (US); Richard Axel, New York, NY (US)

Correspondence Address: John P. White, Cooper & Dunham LLP, 1185 Avenue of the Americas, New York, NY, 10036, US

(21) Appl. No.: 09/771,209

(22) Filed: Jan. 26, 2001

Related U.S. Application Data
(63) Continuation-in-part of application No. 08/129,079, filed on Oct. 5, 1993.

(30) Foreign Application Priority Data
Apr. 6, 1992 (US) PCT/US92/02741

(51) Int. Cl. C12P 21/02; C12N 5/06; C07H 21/04; C07K 14/705

(52) U.S. Cl. 435/69.1; 435/325; 530/350; 536/23.5; 514/12

(57) ABSTRACT

The invention provides an isolated nucleic acid molecule encoding an odorant receptor. The invention also provides expression vectors containing such nucleic acid, purified odorant receptor proteins encoded by the nucleic acid, and transfected cells expressing the receptor proteins. The invention further provides methods of identifying odorant ligands and odorant receptors, of developing fragrances, of identifying appetite suppressant compounds, of controlling appetite, of controlling pest populations, of promoting and inhibiting fertility, and of detecting odors.

This application is a continuation-in-part of U.S. Ser. No. 08/129,079, filed Oct. 5, 1993, a national stage filing of PCT International Application No. PCT/US92/02741, filed Apr. 6, 1992, which claims priority of and is a continuation-in-part of U.S. Ser. No. 07/681,880, filed Apr. 5, 1991, now abandoned, the contents of all of which are hereby incorporated by reference.

The invention disclosed herein was made with Government support under grant number RO1-CA 23767 from the National Institutes of Health, U.S. Department of Health and Human Services. Accordingly, the U.S. Government has certain rights in this invention.

### BACKGROUND OF THE INVENTION

Throughout this application, various publications are referenced by Arabic numerals within parentheses. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein.

In vertebrate sensory systems, peripheral neurons respond to environmental stimuli and transmit these signals to higher sensory centers in the brain where they are processed to allow the discrimination of complex sensory information. The delineation of the peripheral mechanisms by which environmental stimuli are transduced into neural information can provide insight into the logic underlying sensory processing. Our understanding of color vision, for example, emerged only after the observation that the discrimination of hue results from the blending of information from only three classes of photoreceptors (1, 2, 3, 4). The basic logic underlying olfactory sensory perception, however, has remained elusive. Mammals possess an olfactory system of enormous discriminatory power (5, 6). Humans, for example, are thought to be capable of distinguishing among thousands of distinct odors. The specificity of odor recognition is emphasized by the observation that subtle alterations in the molecular structure of an odorant can lead to profound changes in perceived odor.

The detection of chemically distinct odorant presumably results from the association of odorous ligands with specific receptors on olfactory neurons which reside in a specialized epithelium in the nose. Since these receptors have not been identified, it has been difficult to determine how odor discrimination might be achieved. It is possible that olfaction, by analogy with color vision, involves only a few odor receptors, each capable of interaction with multiple odorant molecules. Alternatively, the sense of smell may involve a large number of distinct receptors each capable of associating with one or a small number of odorant. In either case, the brain must distinguish which receptors or which neurons have been activated to allow the discrimination between different odorant stimuli. Insight into the mechanisms underlying olfactory perception is likely to depend upon the isolation of the odorant receptors, and the characterization of their diversity, specificity, and patterns of expression.

The primary events in odor detection occur in a specialized olfactory neuroepithelium located in the posterior recesses of the nasal cavity. Three cell types dominate this epithelium (FIG. 1A): the olfactory sensory neuron, the sustentacular or supporting cell, and the basal cell which is a stem cell that generates olfactory neurons throughout life (7, 8). The olfactory sensory neuron is bipolar: a dendritic process extends to the mucosal surface where it gives rise to a number of specialized cilia which provide an extensive, receptive surface for the interaction of odors with olfactory sensory neurons. The olfactory neuron also gives rise to an axon which projects to the olfactory bulb of the brain, the first relay in the olfactory system. The axons of the olfactory bulb neurons, in turn, project to subcortical and cortical regions where higher level processing of olfactory information allows the discrimination of odors by the brain.

The initial events in odor discrimination are thought to involve the association of odors with specific receptors on the cilia of olfactory neurons. Selective removal of the cilia results in the loss of olfactory response (9). Moreover, in fish, whose olfactory system senses amino acids as odors, the specific binding of amino acids to isolated cilia has been demonstrated (10, 11). The cilia are also the site of olfactory signal transduction. Exposure of isolated cilia from rat olfactory epithelium to numerous odorant leads to the rapid stimulation of adenylyl cyclase and elevations in cyclic AMP (an elevation in IP3 in response to one odorant has also been observed) (12, 13, 14, 15). The activation of adenylyl cyclase is dependent on the presence of GTP and is therefore likely to be mediated by receptor-coupled GTP binding proteins (G-proteins) (16). Elevations in cyclic AMP, in turn, are thought to elicit depolarization of olfactory neurons by direct activation of a cyclic nucleotide-gated, cation permeable channel (17, 18). This channel is opened upon binding of cyclic nucleotides to its cytoplasmic domain, and can therefore transduce changes in intracellular levels of cyclic AMP into alterations in the membrane potential.

These observations suggest a pathway for olfactory signal transduction (FIG. 1B) in which the binding of odors to specific surface receptors activates specific G-proteins. The G-proteins then initiate a cascade of intracellular signaling events leading to the generation of an action potential which is propagated along the olfactory sensory axon to the brain. A number of neurotransmitter and hormone receptors which transduce intracellular signals by activation of specific G-proteins have been identified. Gene cloning has demonstrated that each of these receptors is a member of a large superfamily of surface receptors which traverse the membrane seven times (19, 20). The pathway of olfactory signal transduction (FIG. 1B) predicts that the odorant receptors might also be members of this superfamily of receptor proteins. The detection of odors in the periphery is therefore likely to involve signaling mechanisms shared by other hormone or neurotransmitter systems, but the vast discriminatory power of the olfactory system will require higher order neural processing to permit the perception of individual odors. This invention address the problem of olfactory perception at a molecular level. Eighteen different members of an extremely large multigene family have been cloned and characterized which encode seven transmembrane domain proteins whose expression is restricted to the olfactory epithelium. The members of this novel gene family encode the individual odorant receptors.

### SUMMARY OF THE INVENTION

The invention provides an isolated nucleic acid, e.g. a DNA and cDNA molecule, encoding an odorant receptor. The invention further provides expression vectors containing such nucleic acid. Also provided by the invention is a purified odorant receptor protein encoded by the isolated nucleic acid. The invention further provides a method of transforming cells which comprises transfecting a suitable host cell with a suitable expression vector containing the nucleic acid encoding the odorant receptor.

The invention also provides methods of identifying odorant ligands and of identifying odorant receptors. The invention further provides methods of developing fragrances, of identifying appetite suppressant compounds, and of controlling appetite. The invention also provides methods of controlling animal populations. The invention additionally provides a method of detecting odors such as the vapors emanating from cocaine, marijuana, heroin, hashish, angel dust, gasoline, decayed human flesh, alcohol, gun powder explosives, plastic explosives, firearms, poisonous or harmful smoke, or natural gas.

### DESCRIPTION OF THE FIGURES

FIGS. 1A-B. The Olfactory Neuroepithelium and a Pathway for Olfactory Signal Transduction.

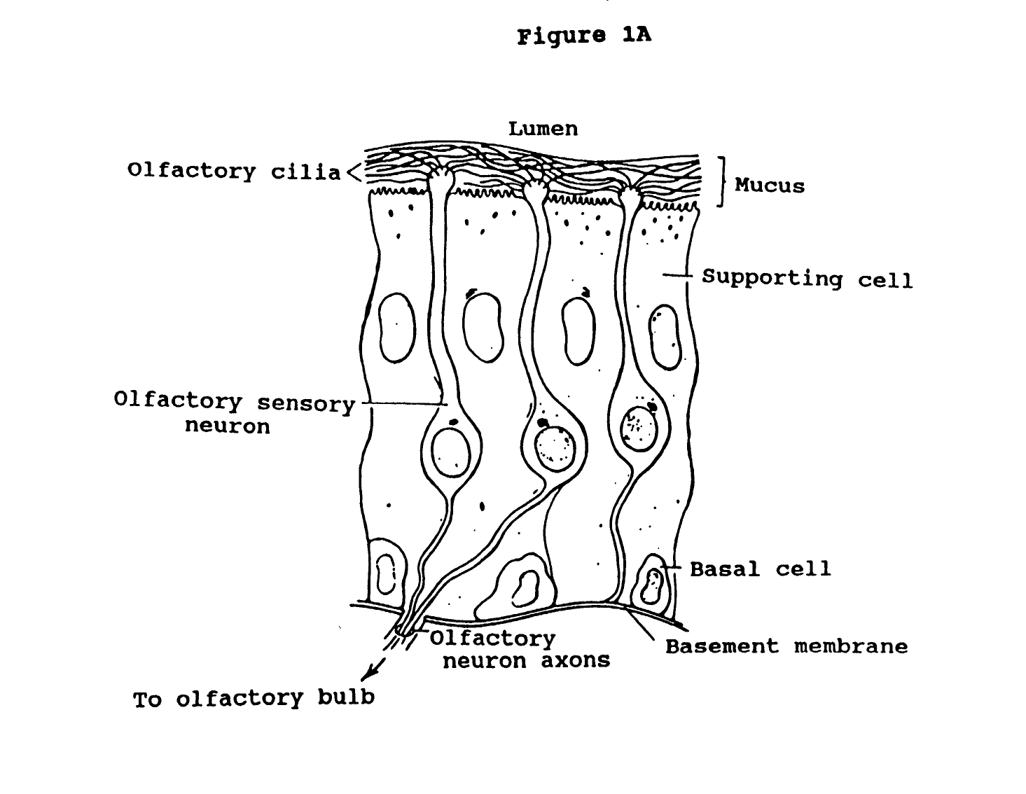

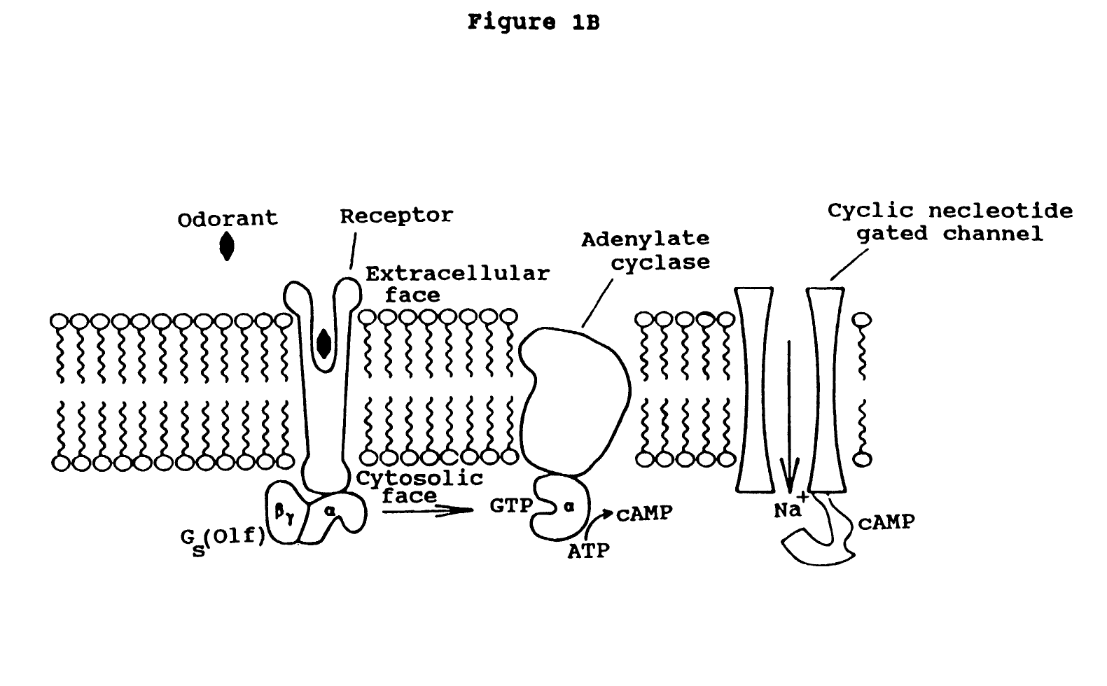

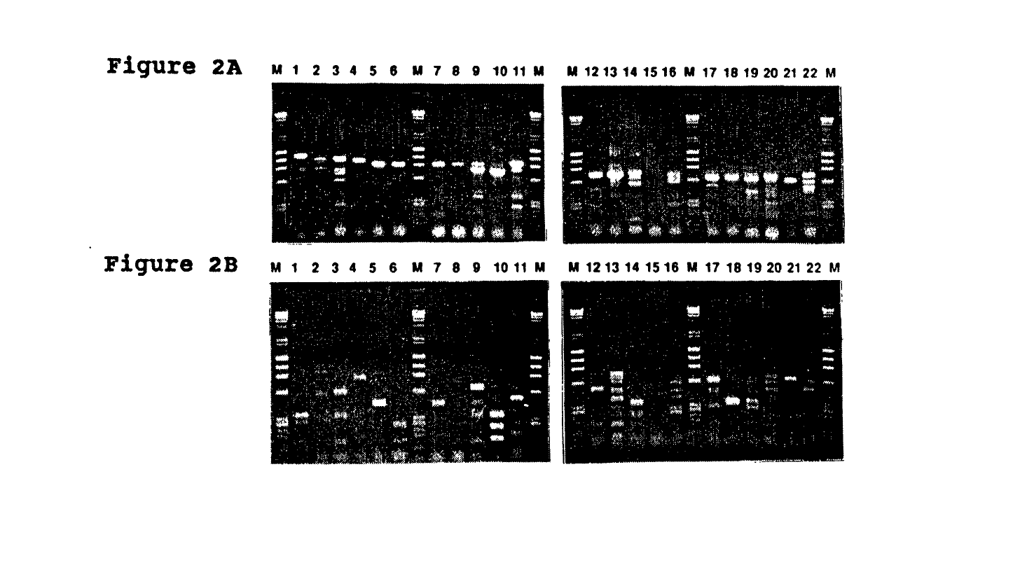

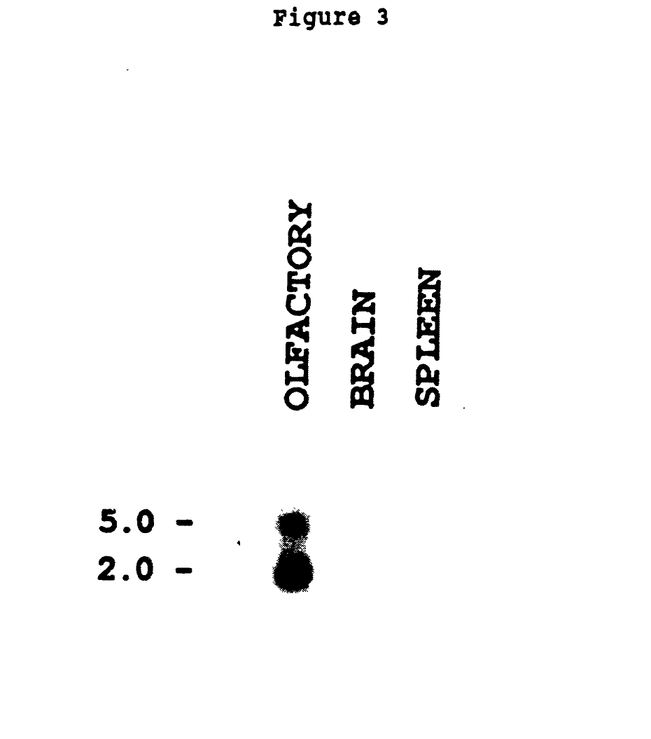

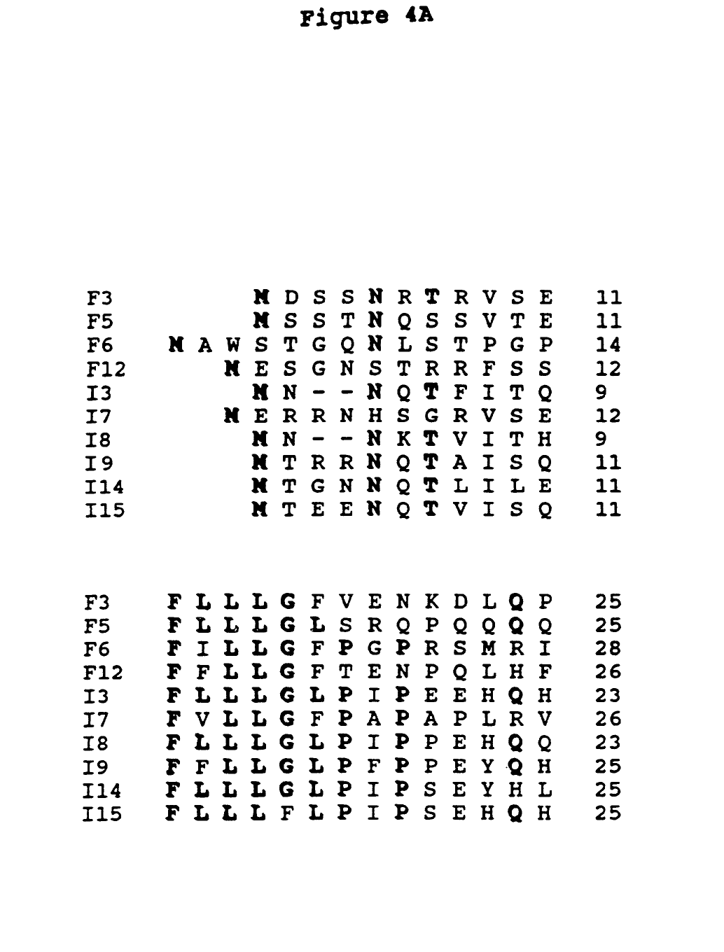

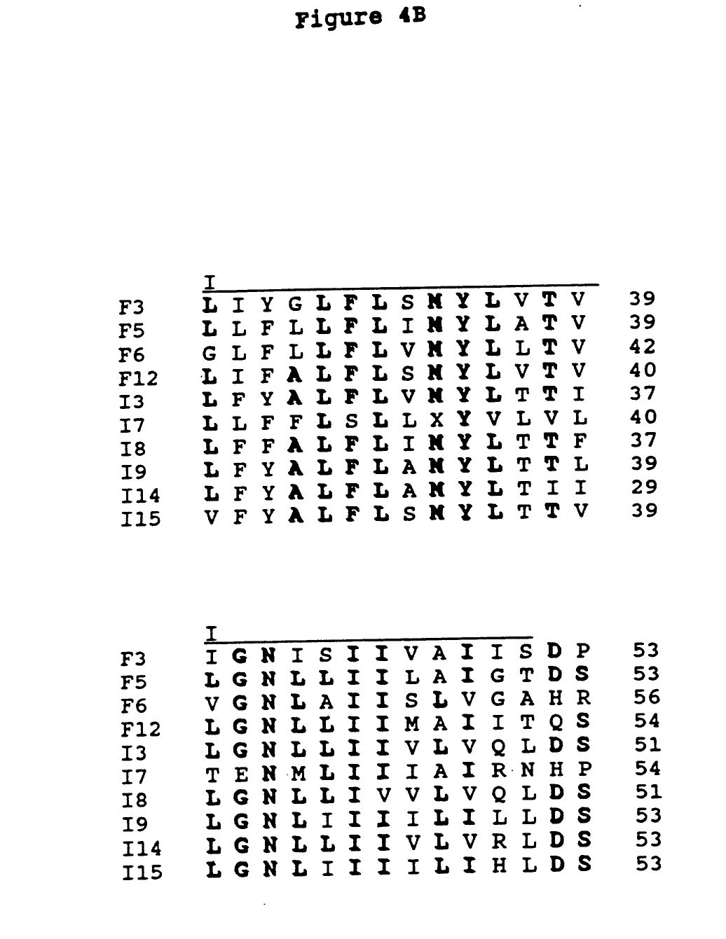

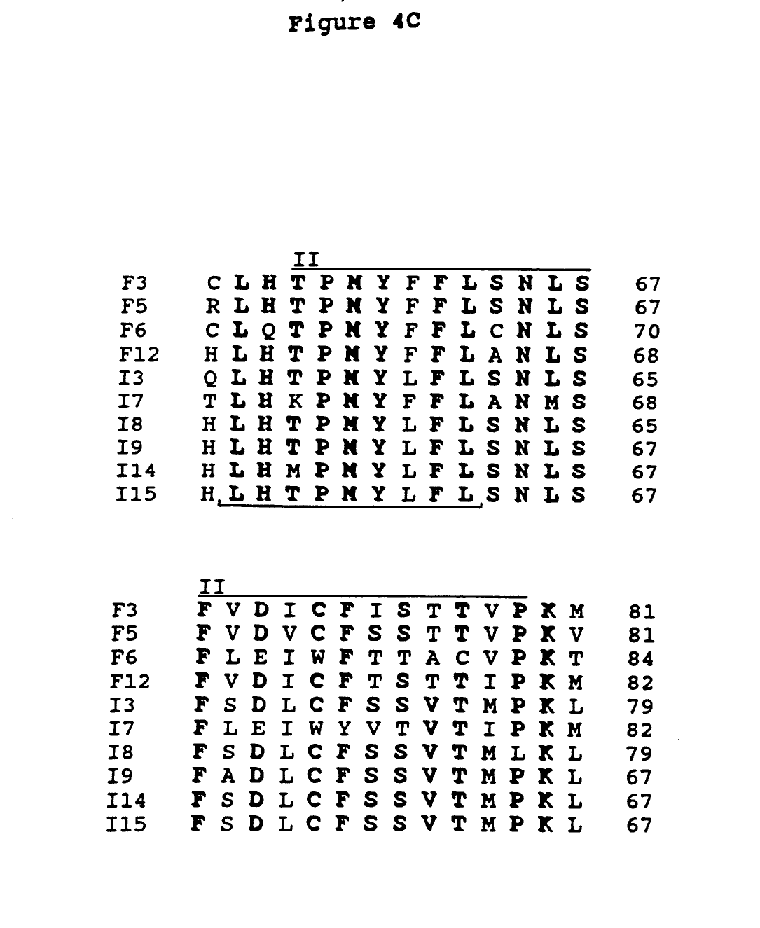

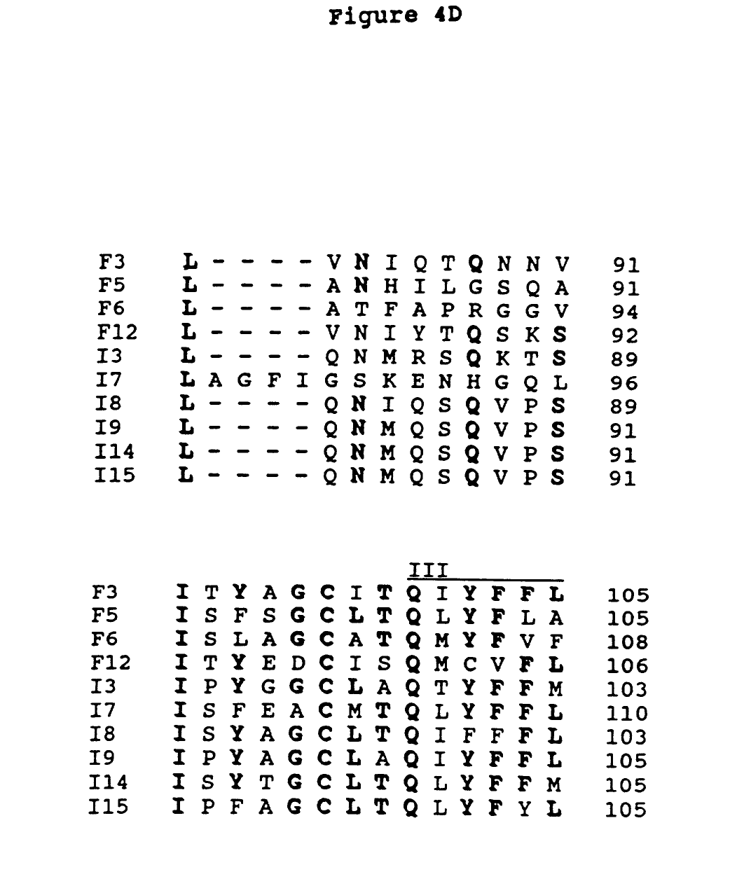

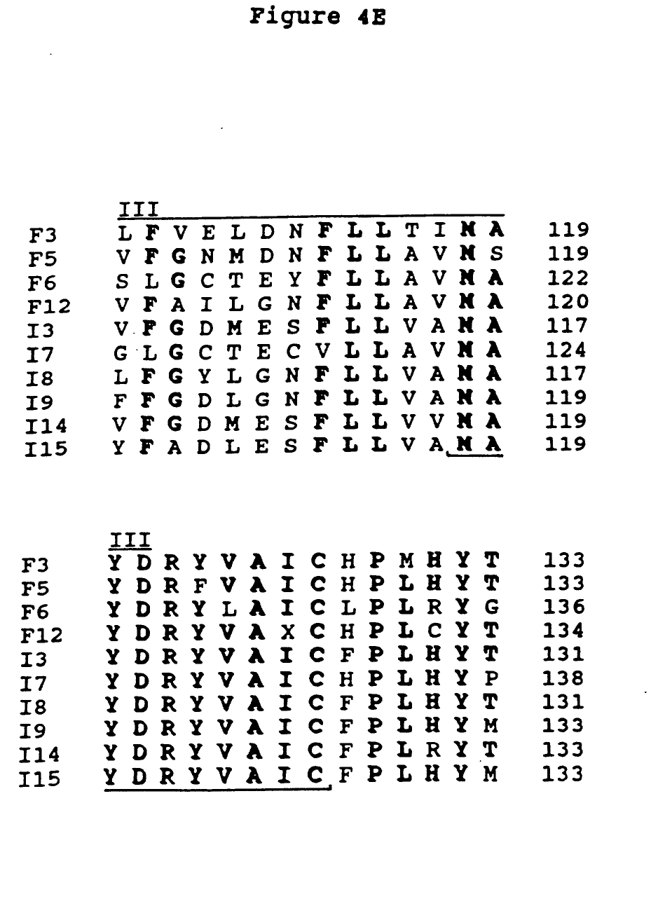

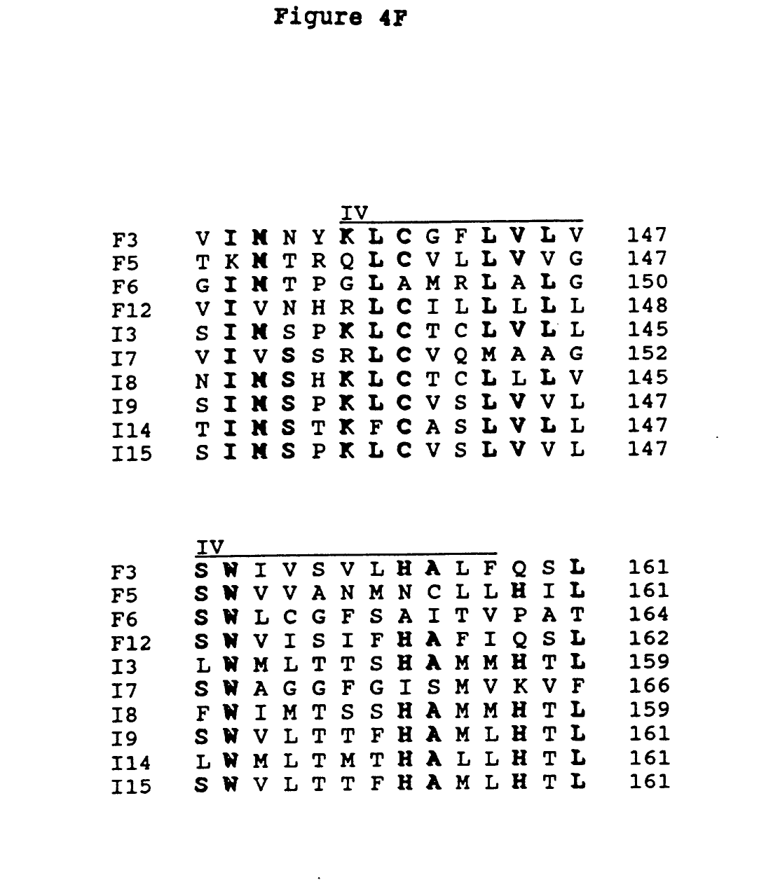

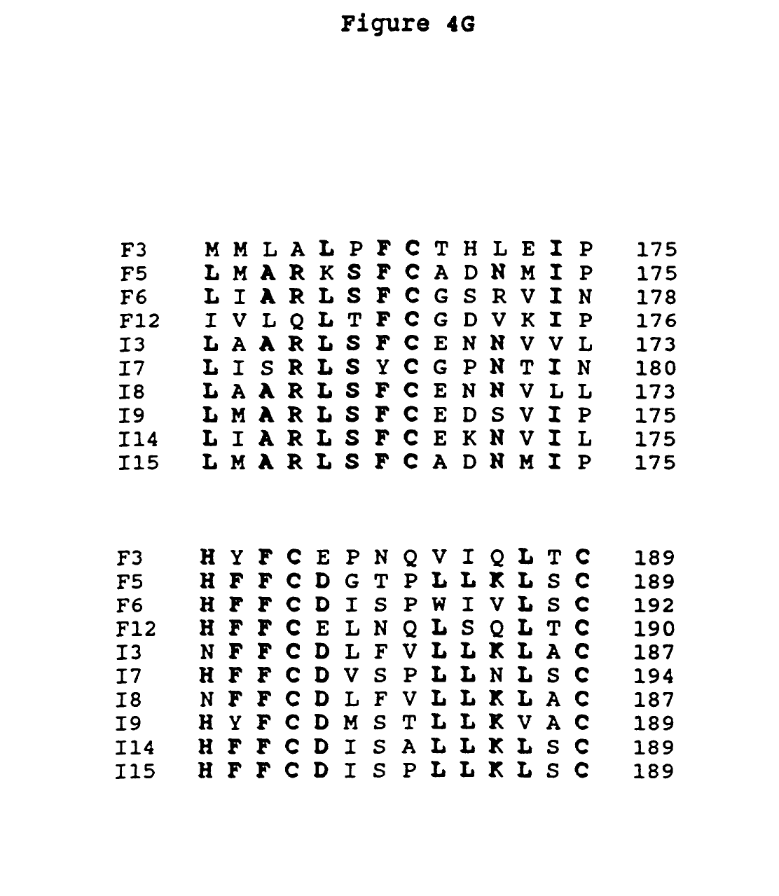

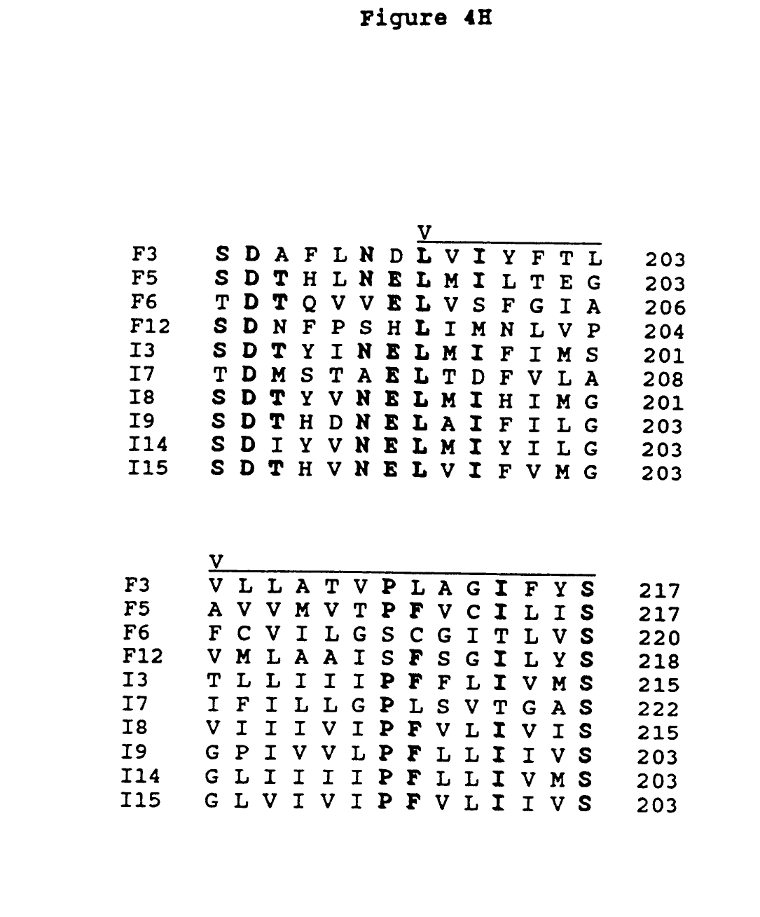

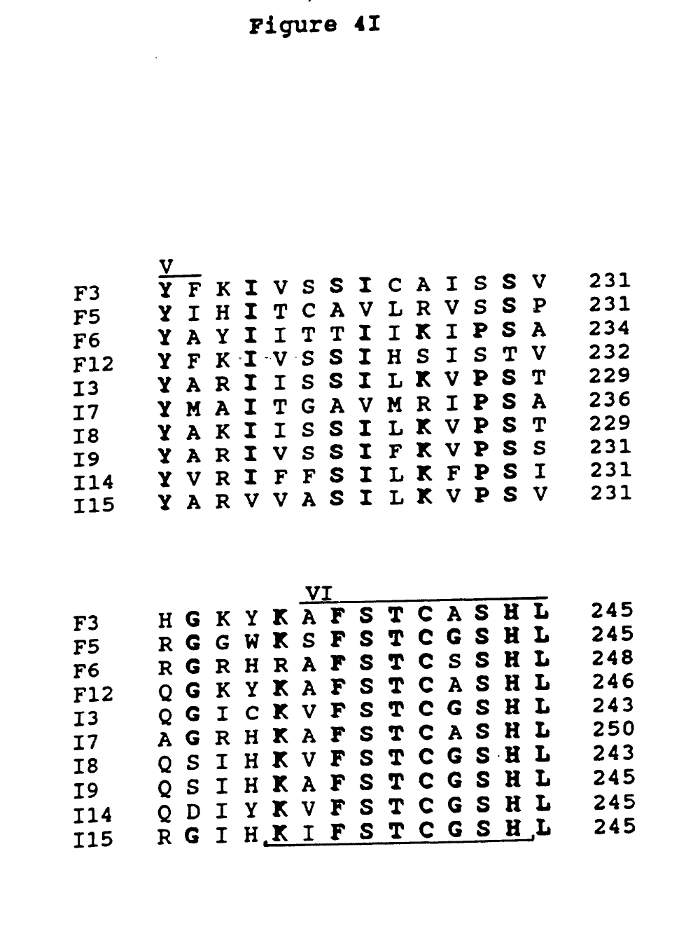

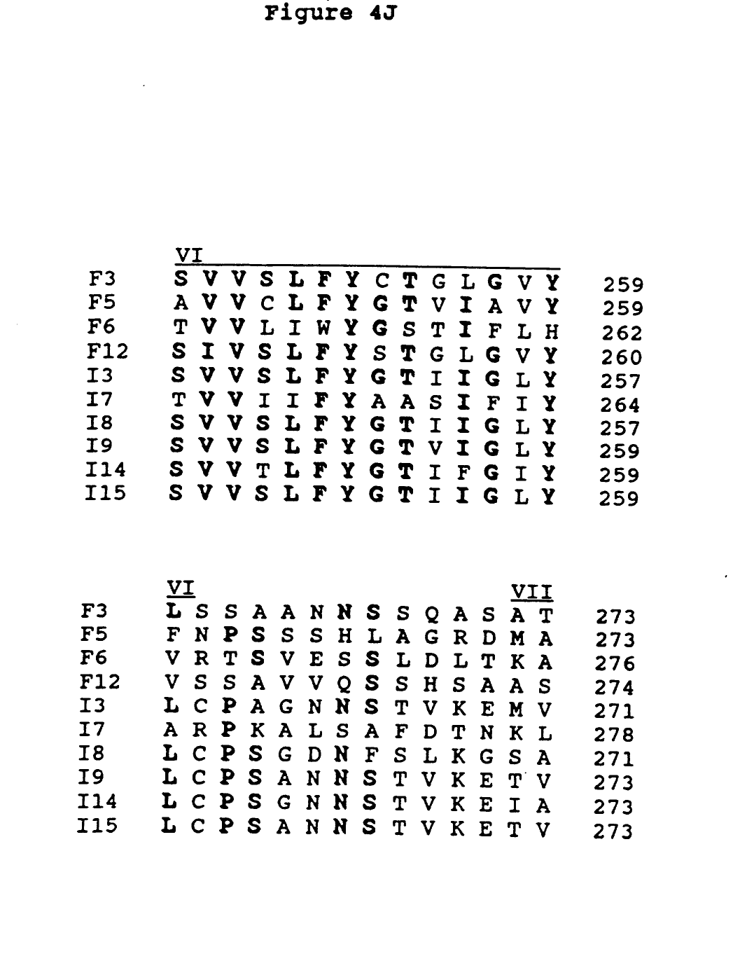

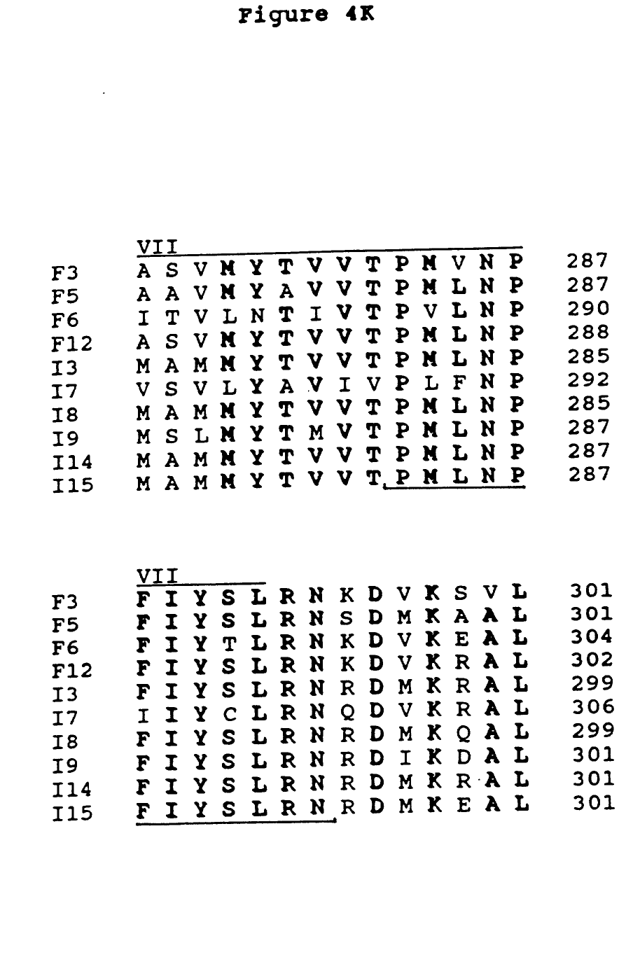

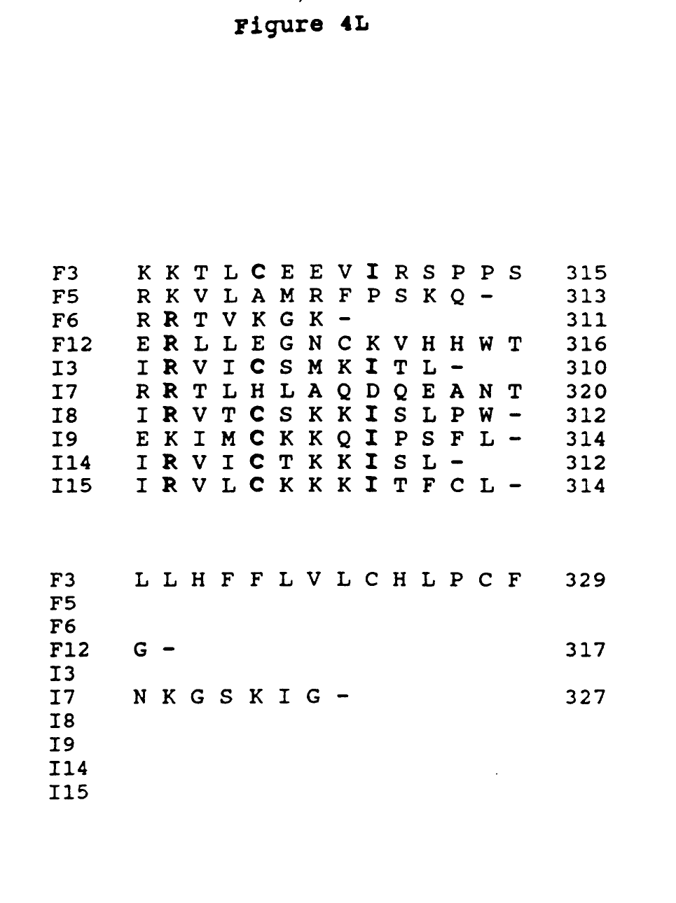

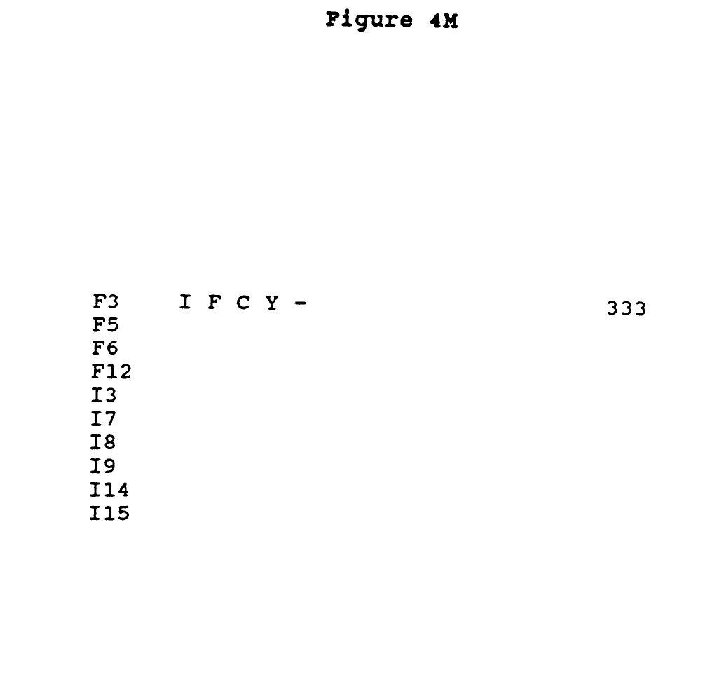

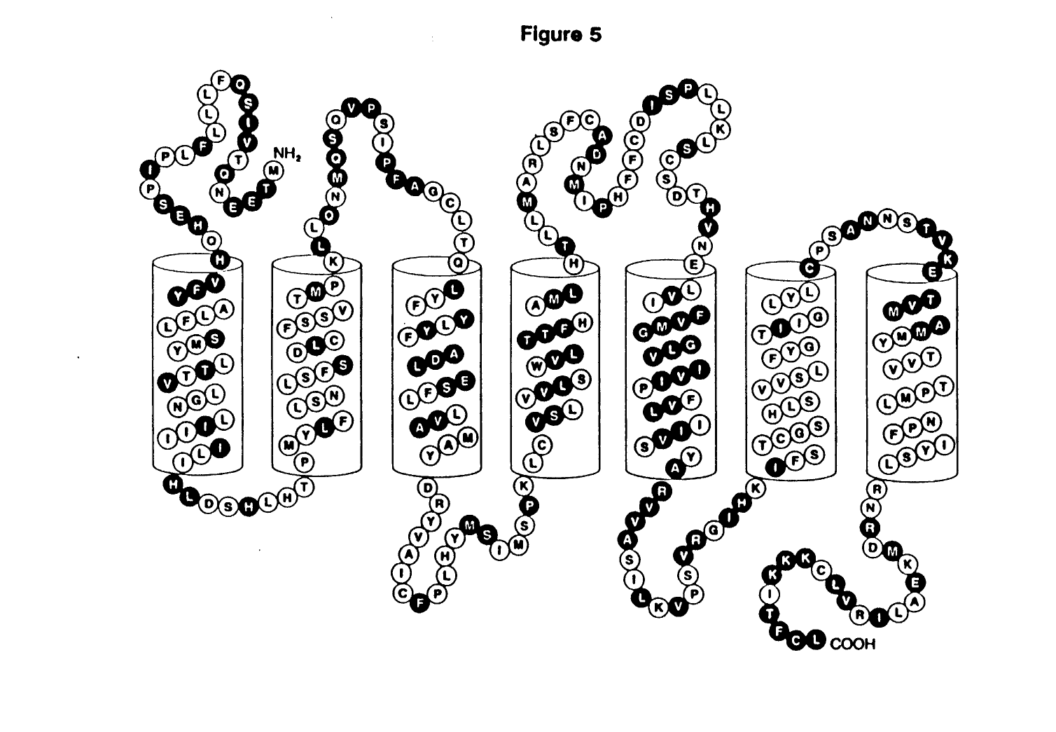

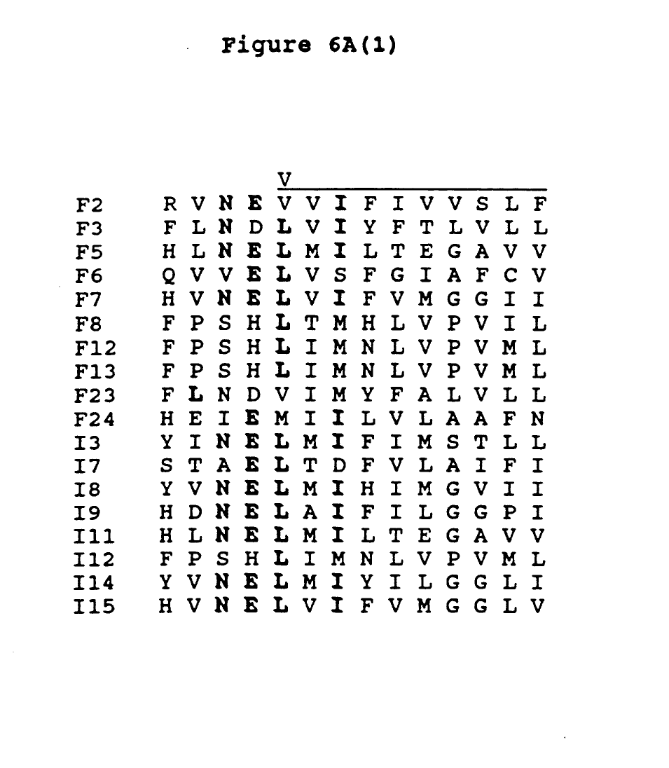

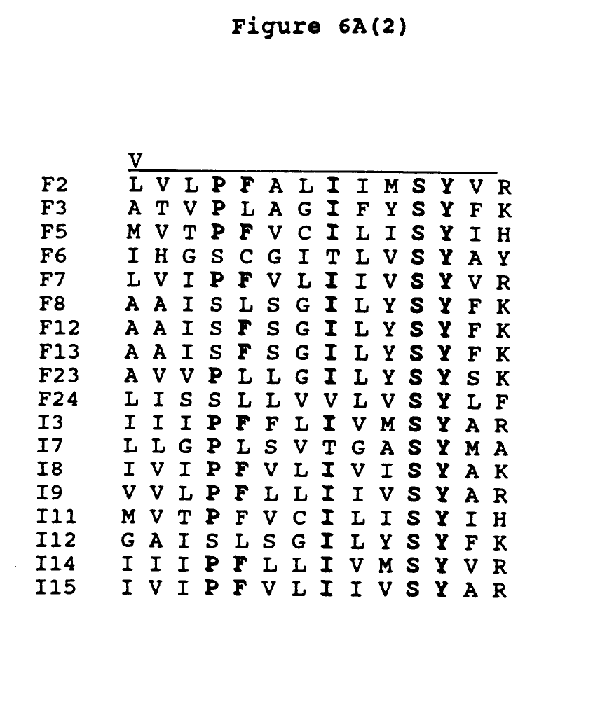

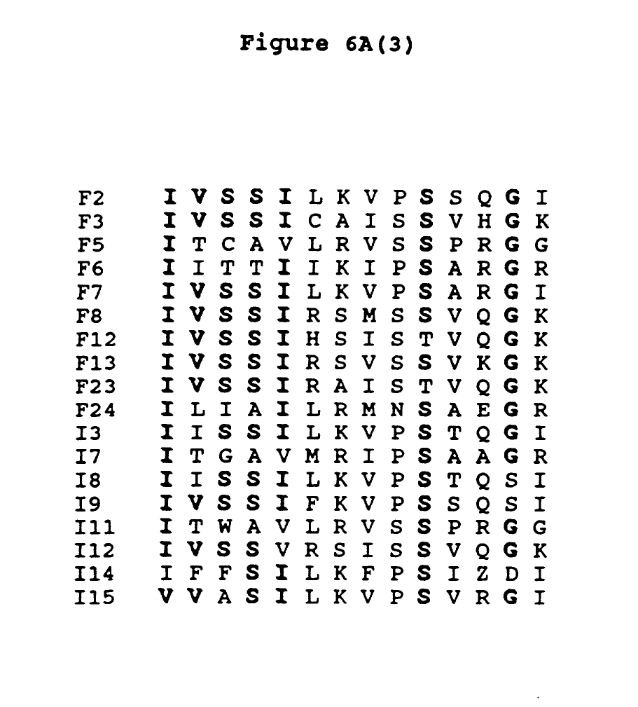

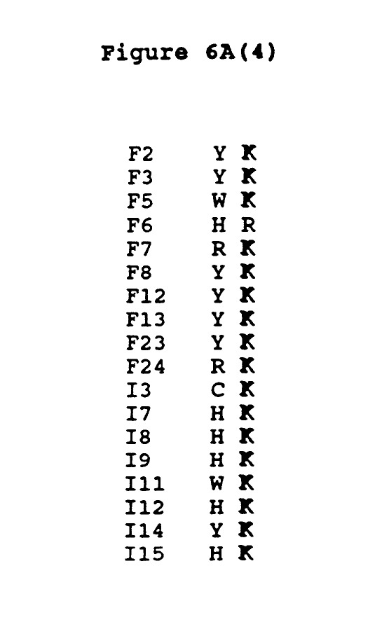

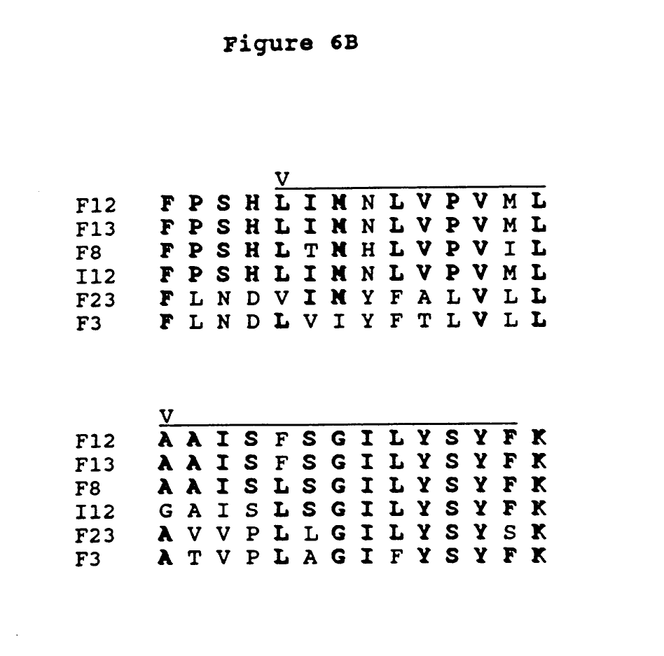

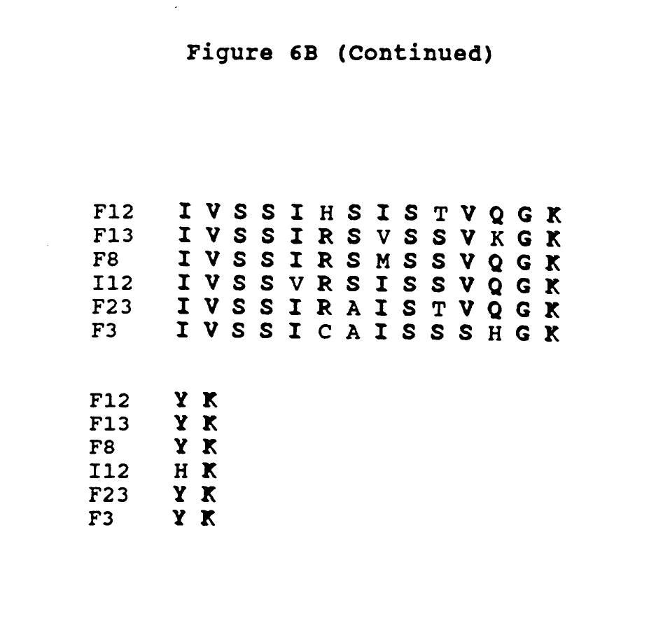

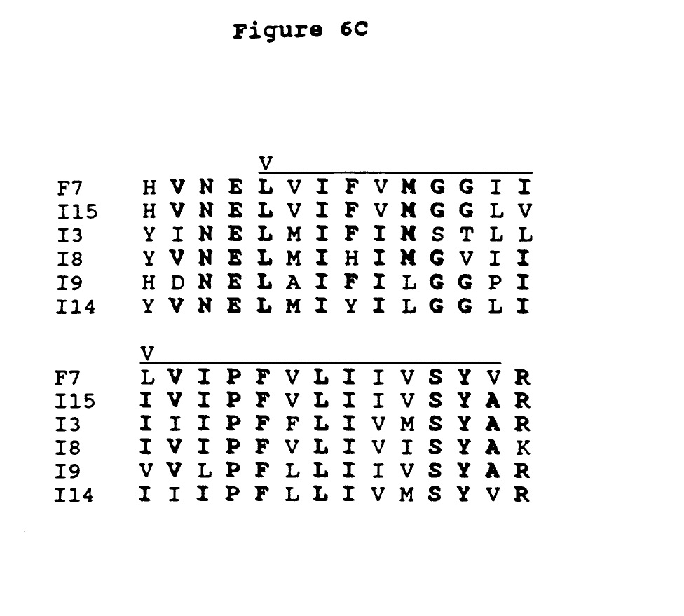

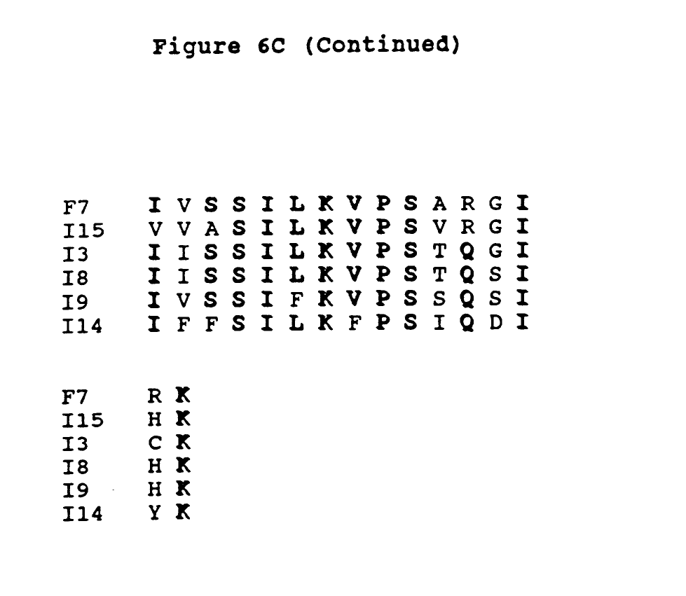

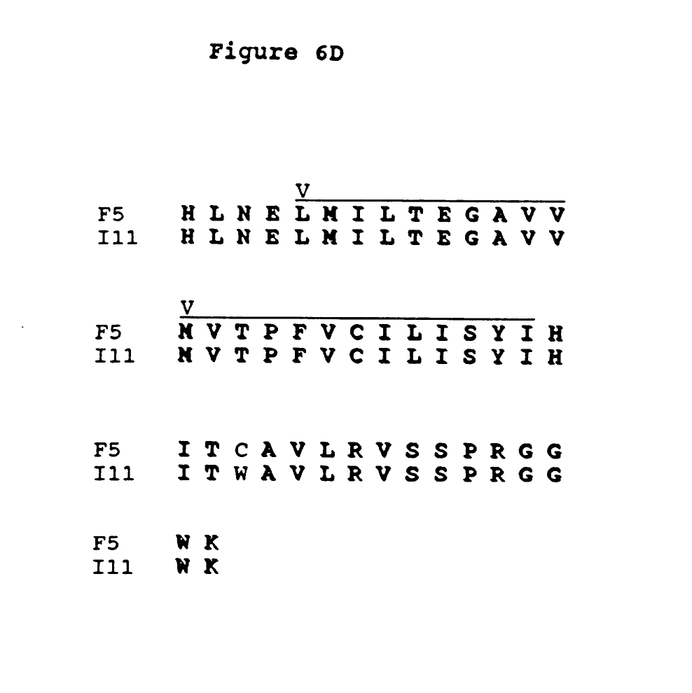

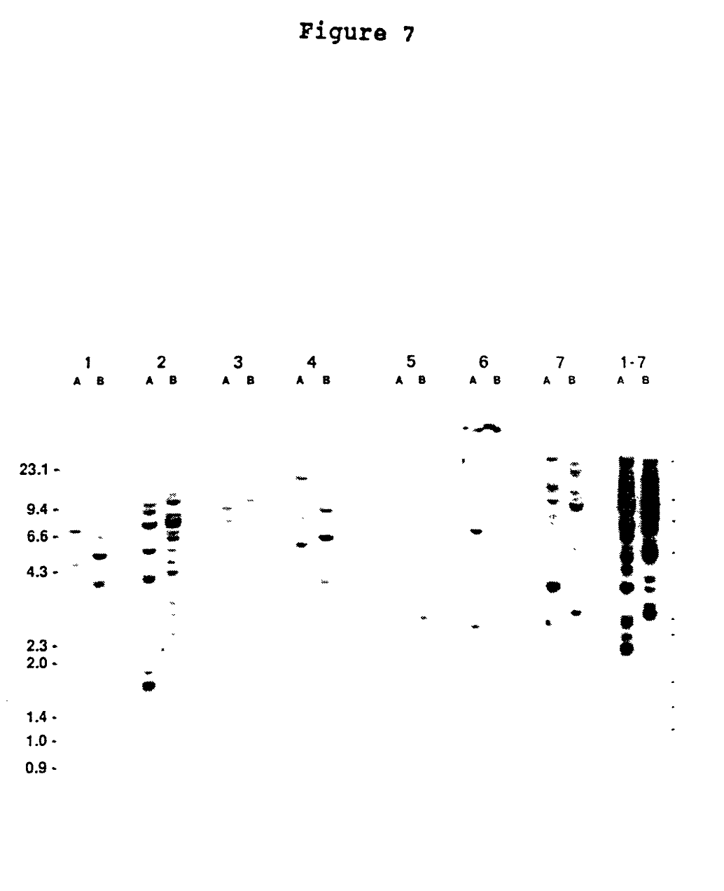

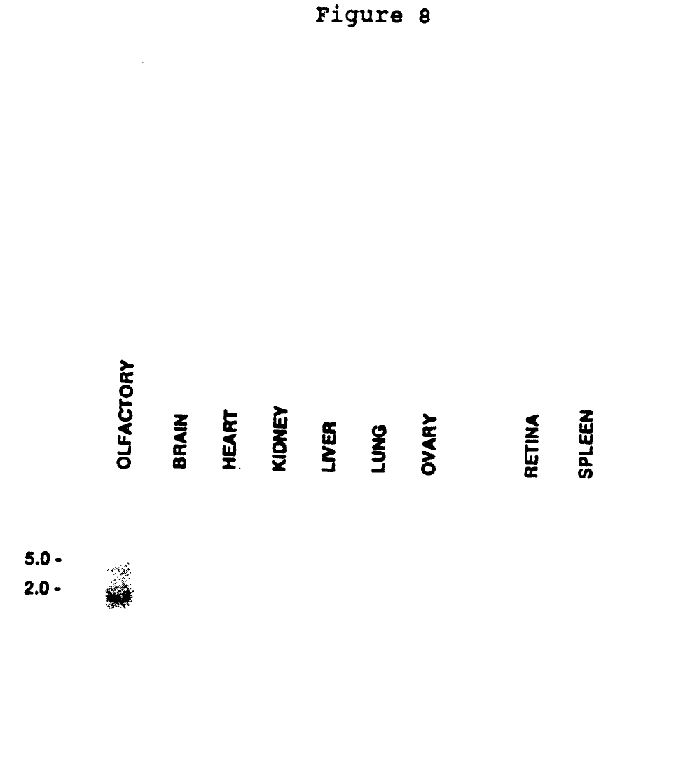

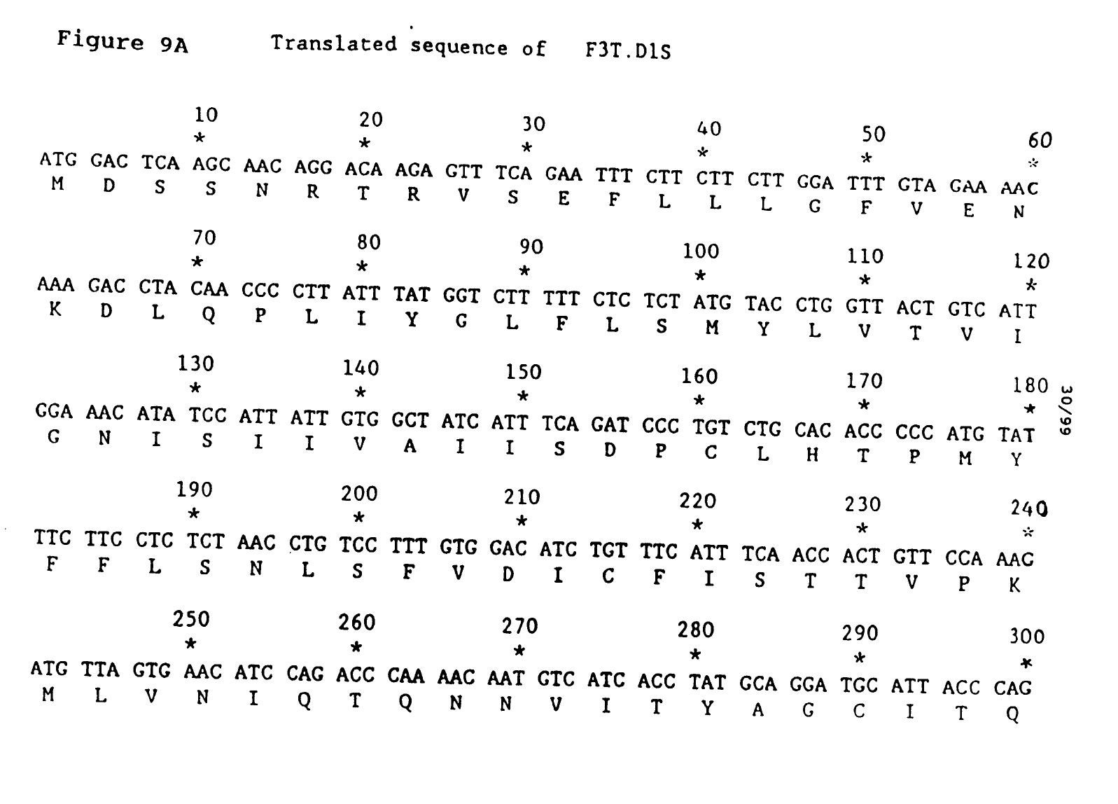

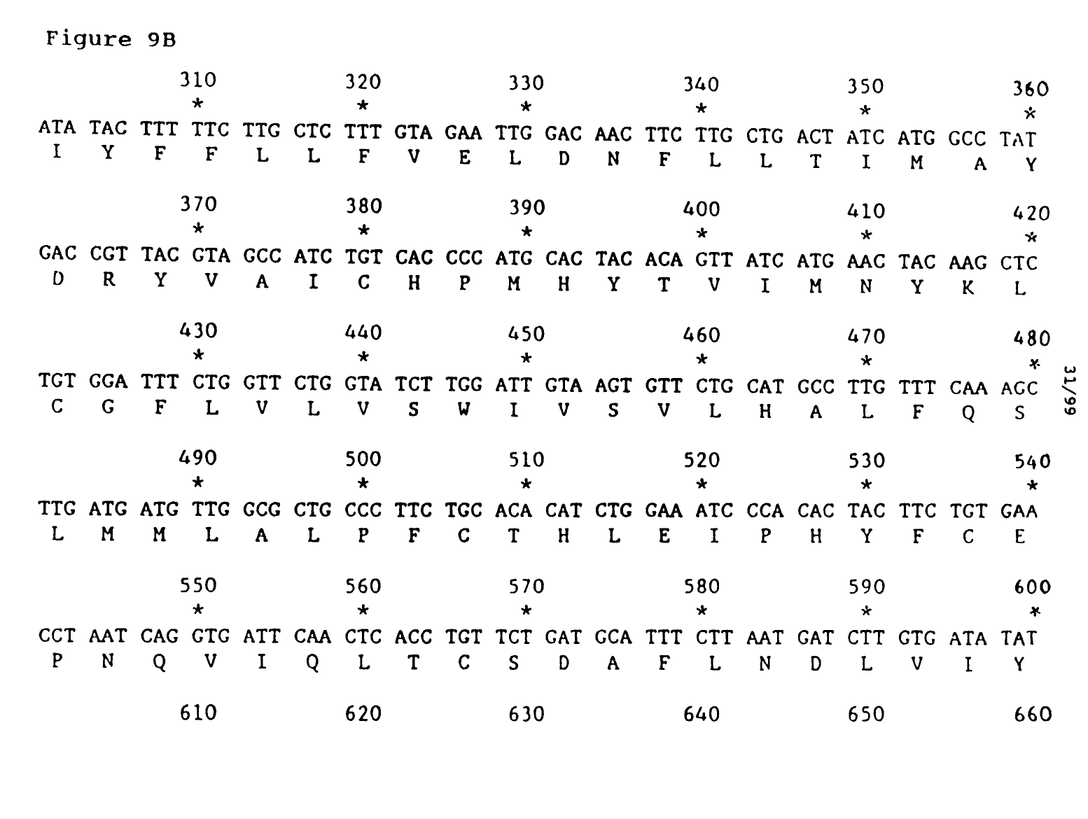

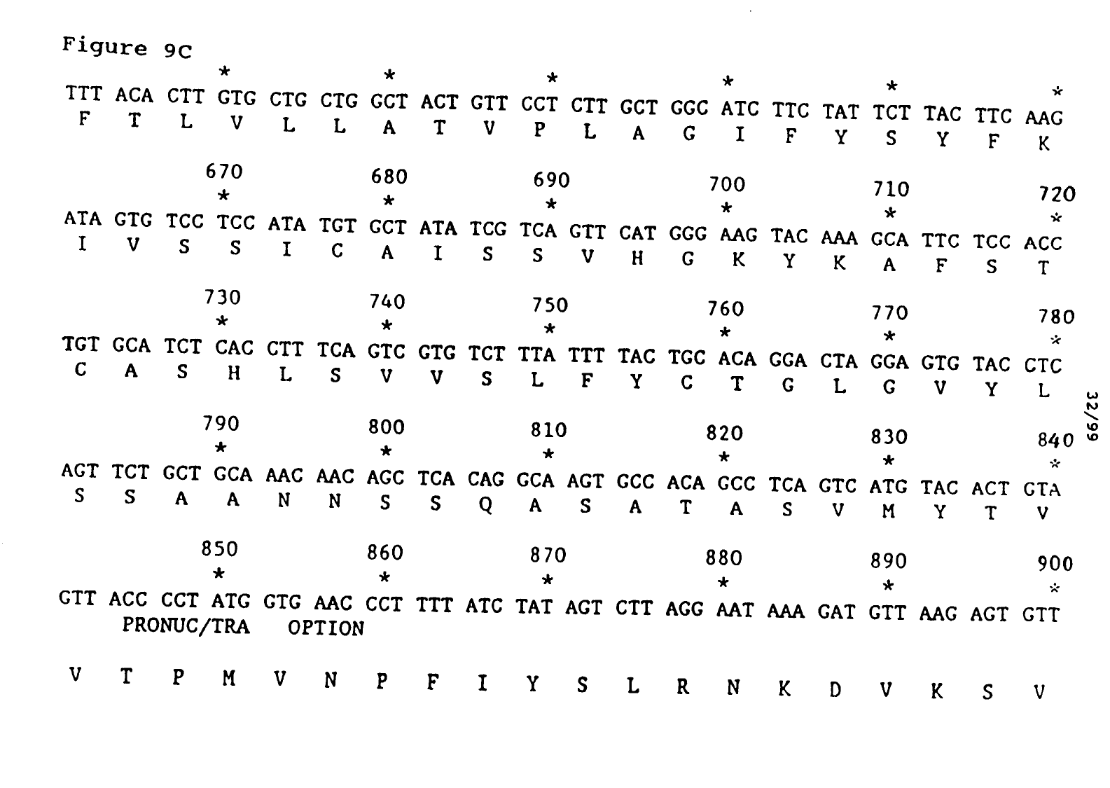

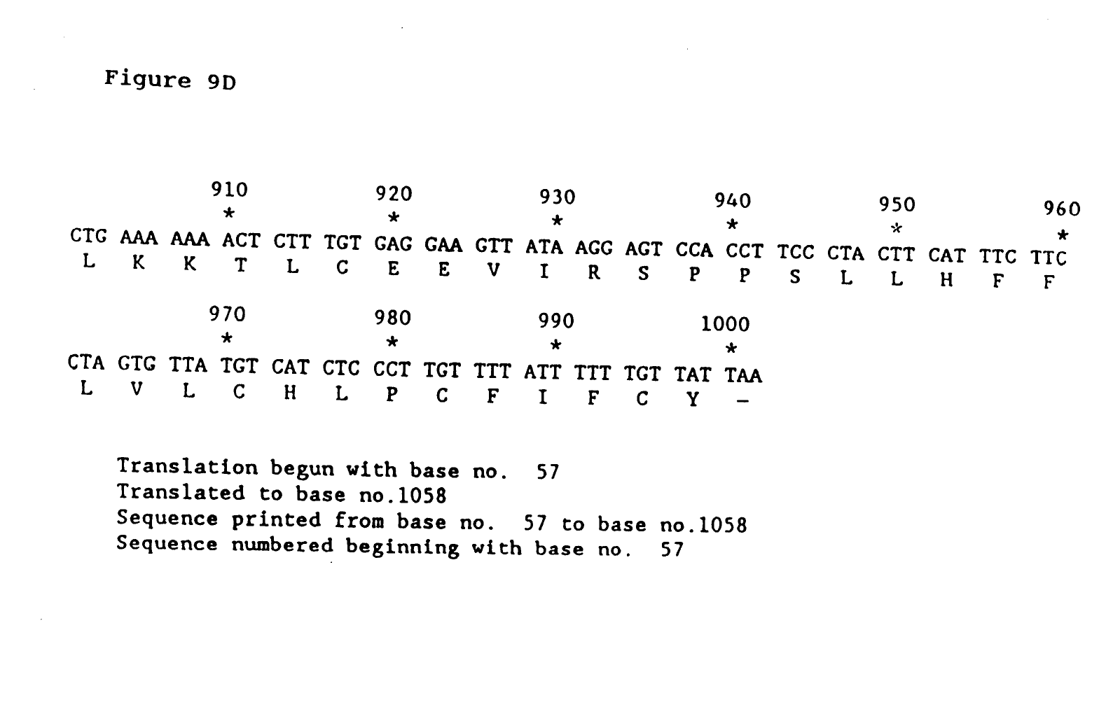

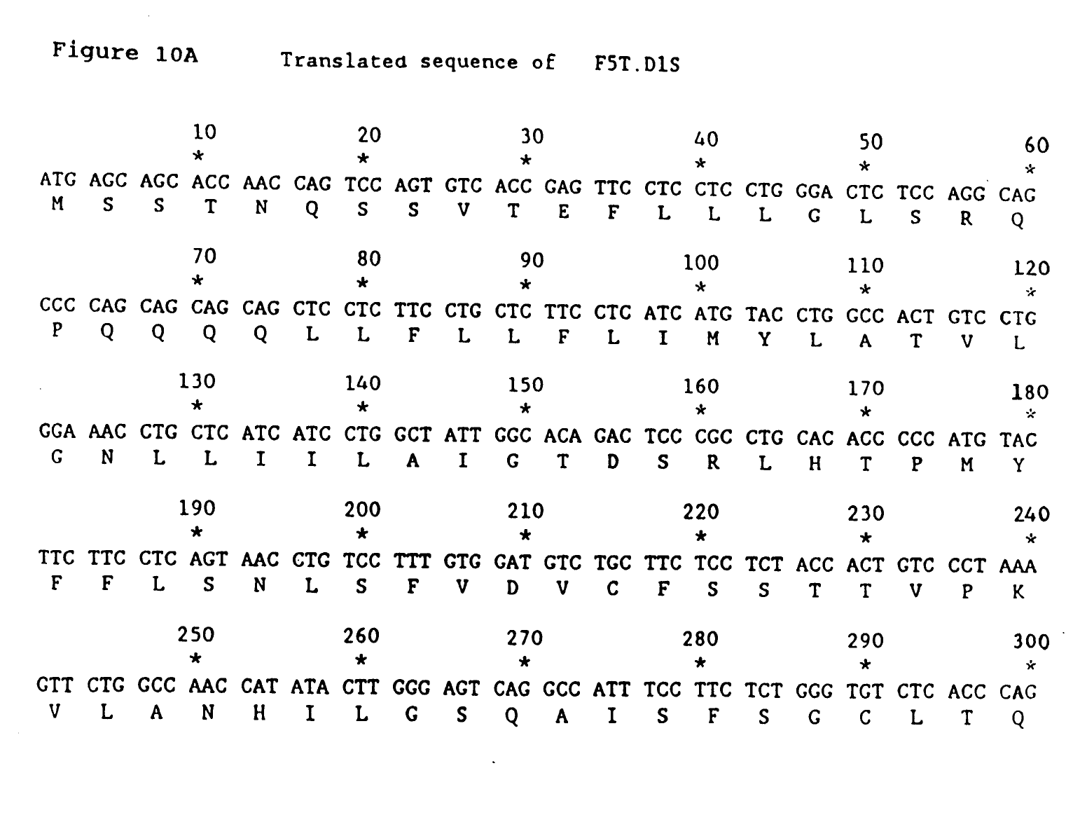

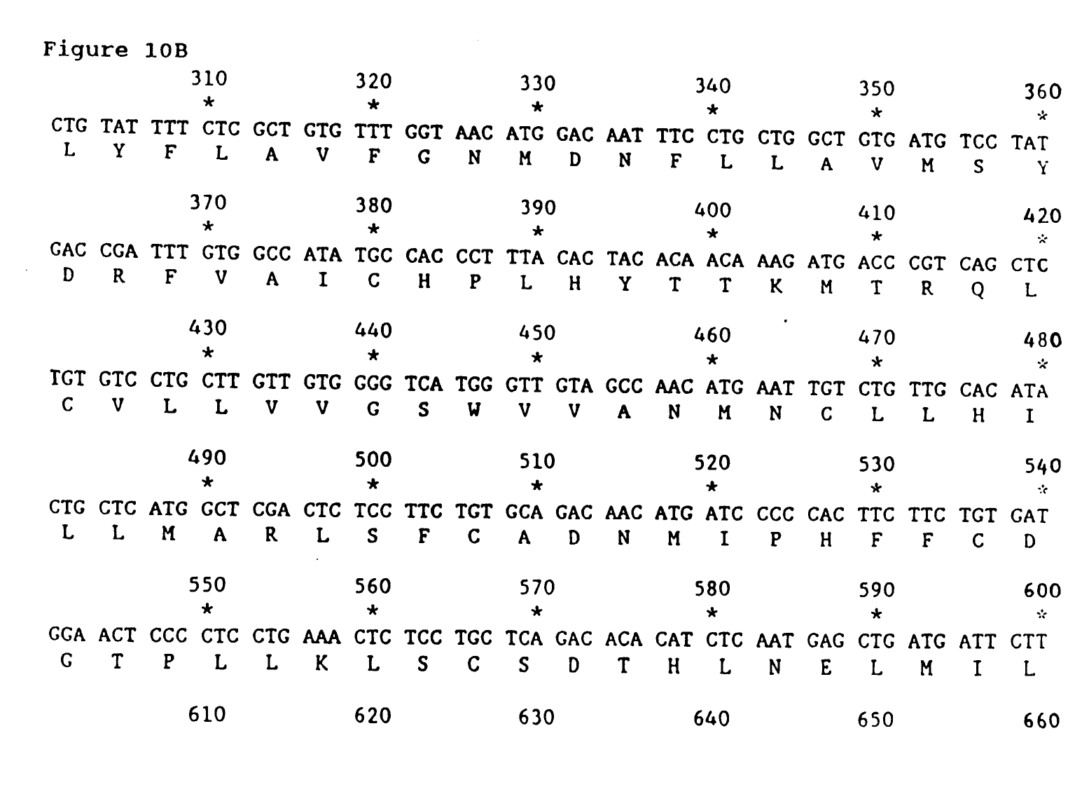

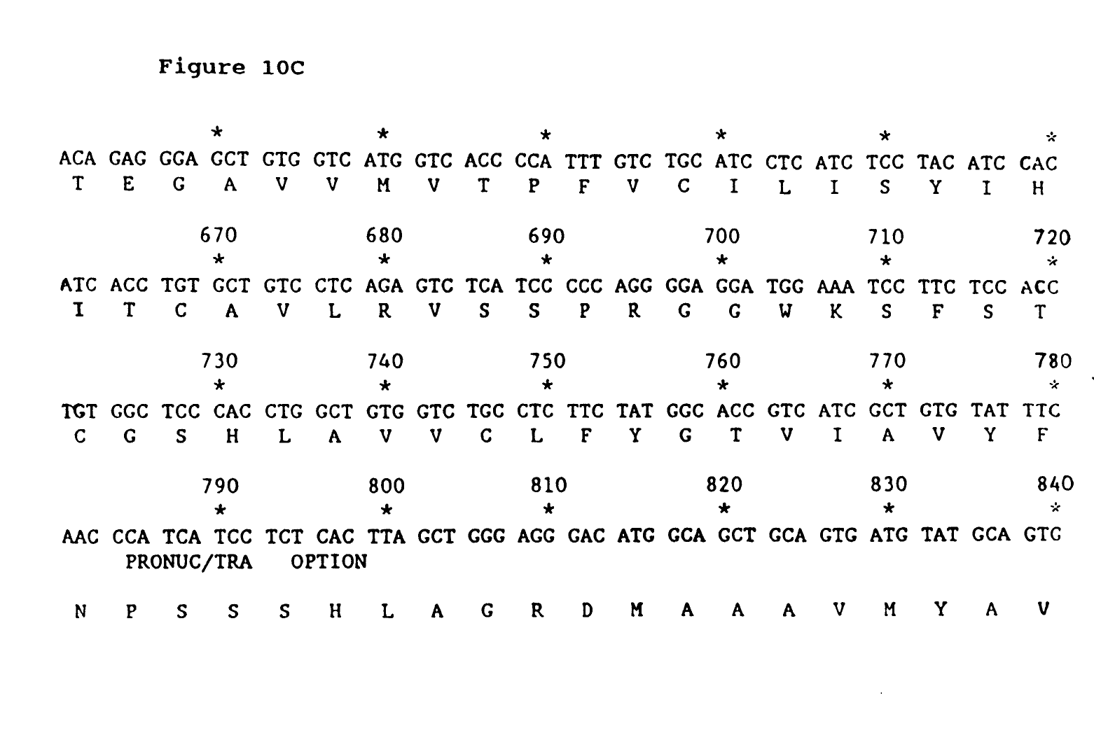

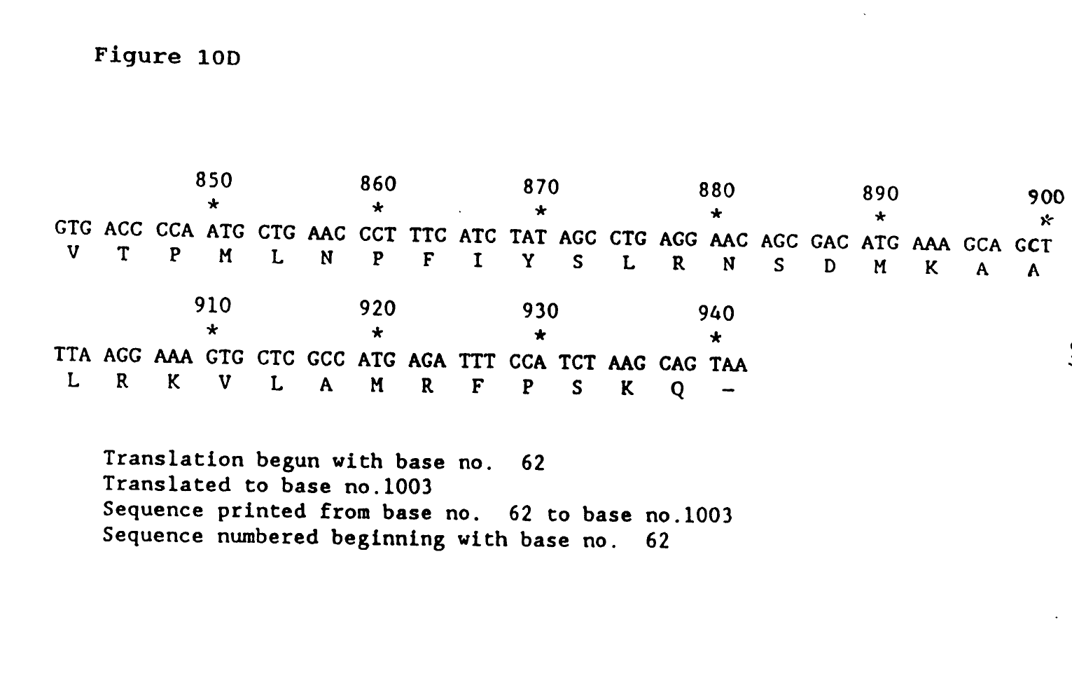

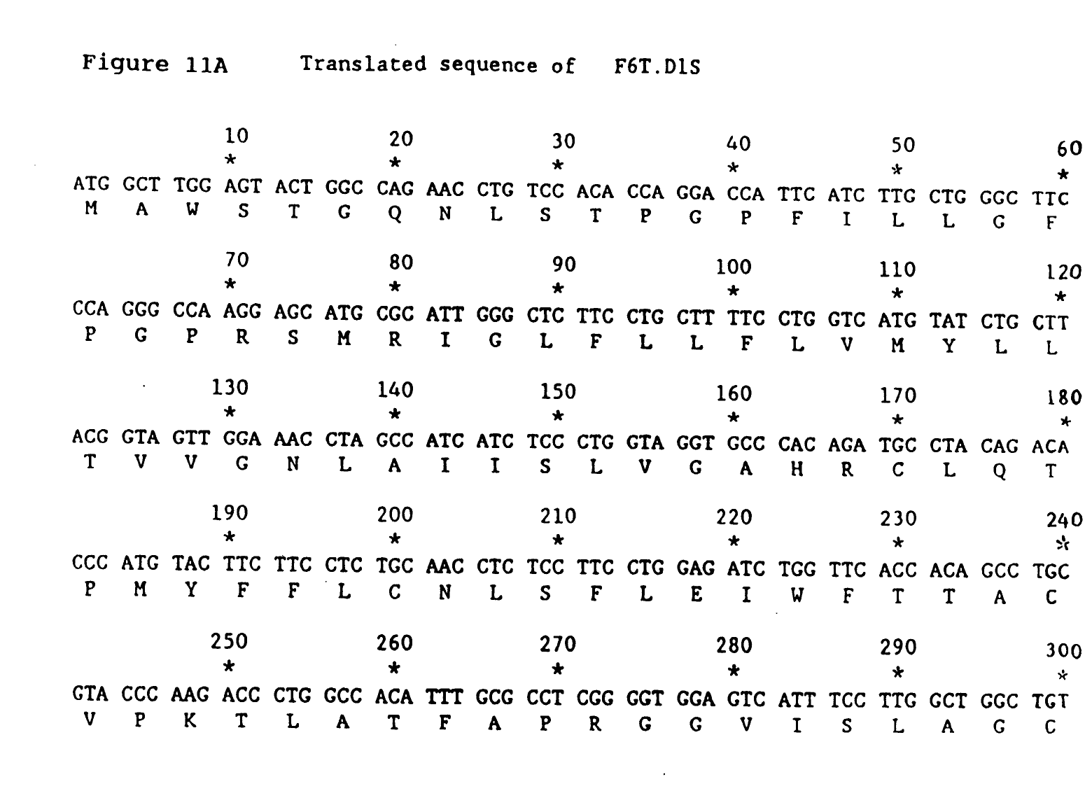

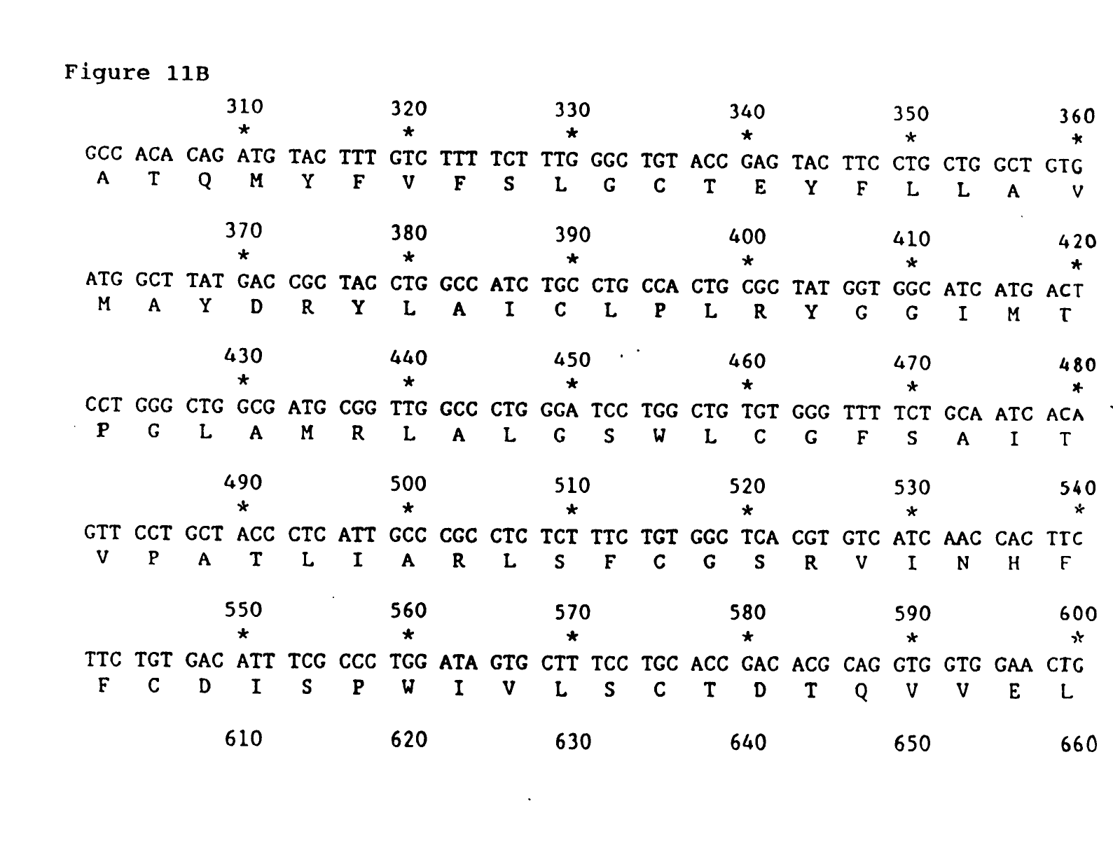

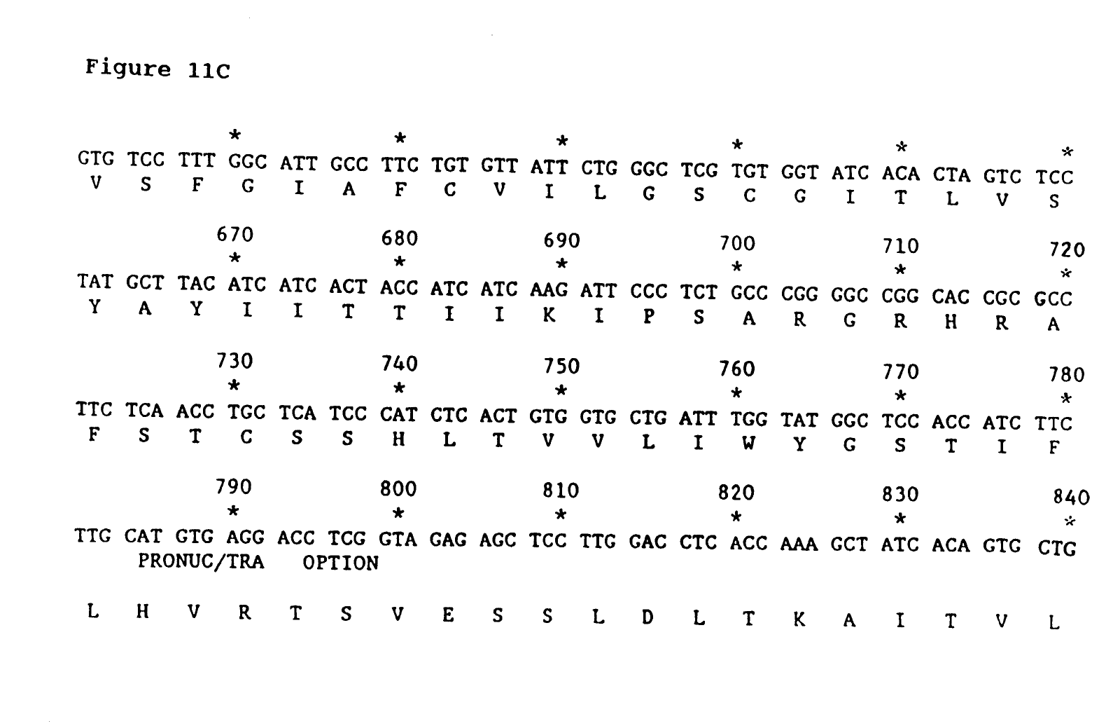

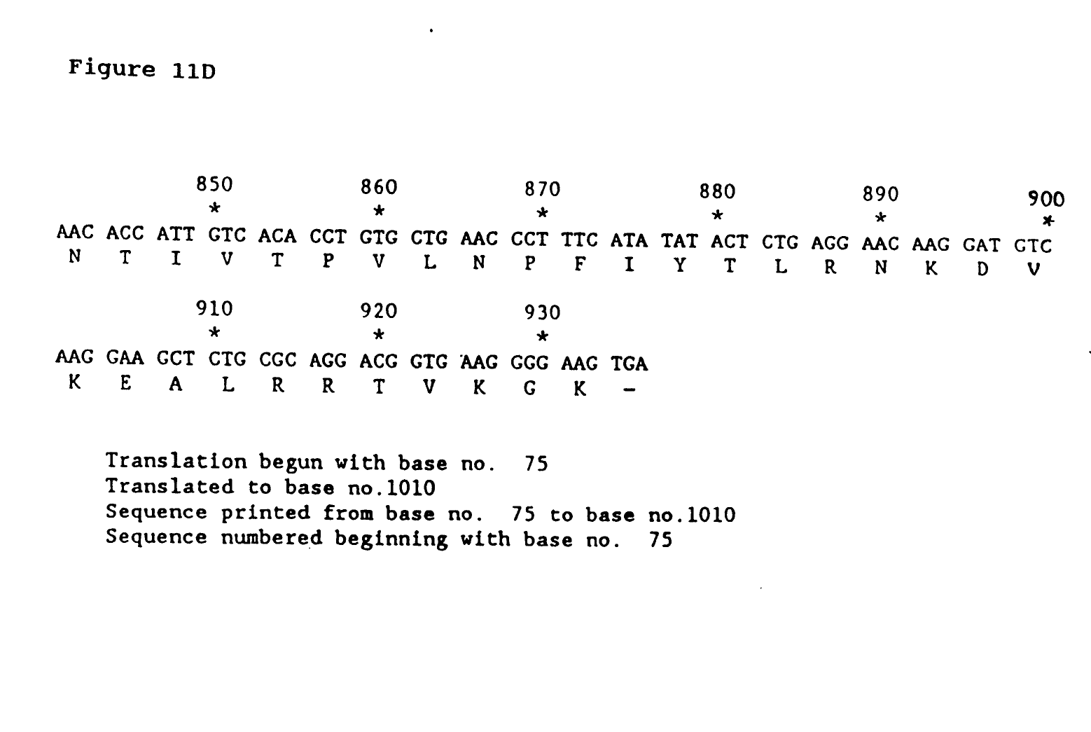

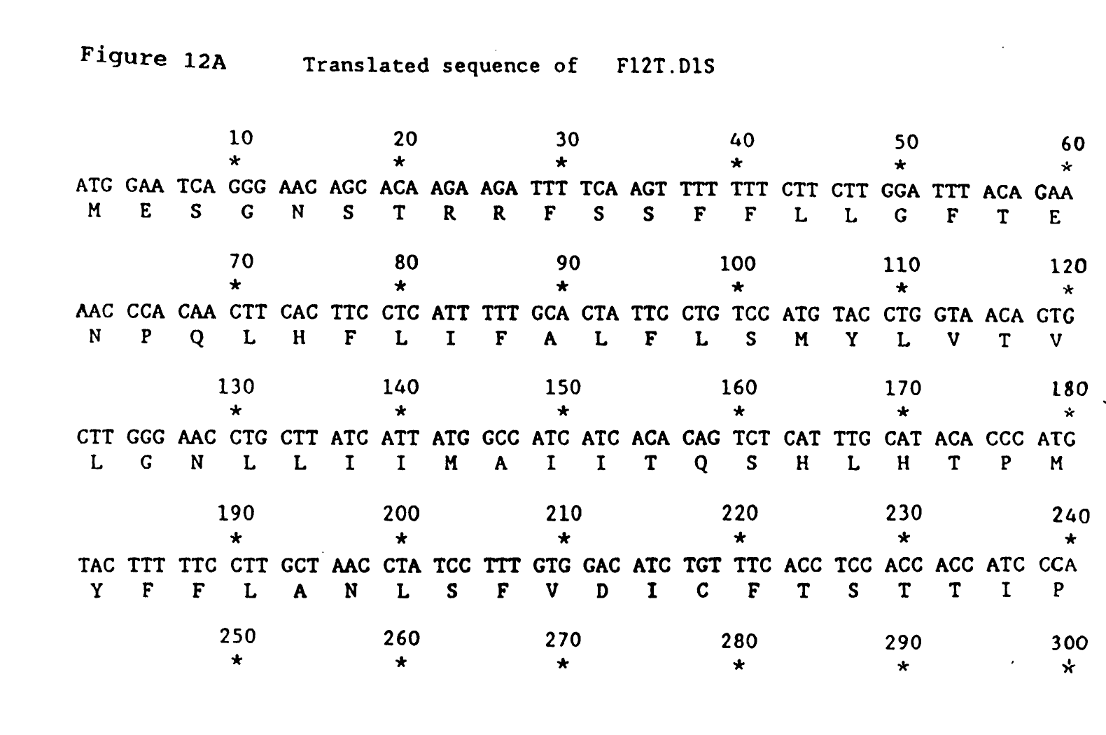

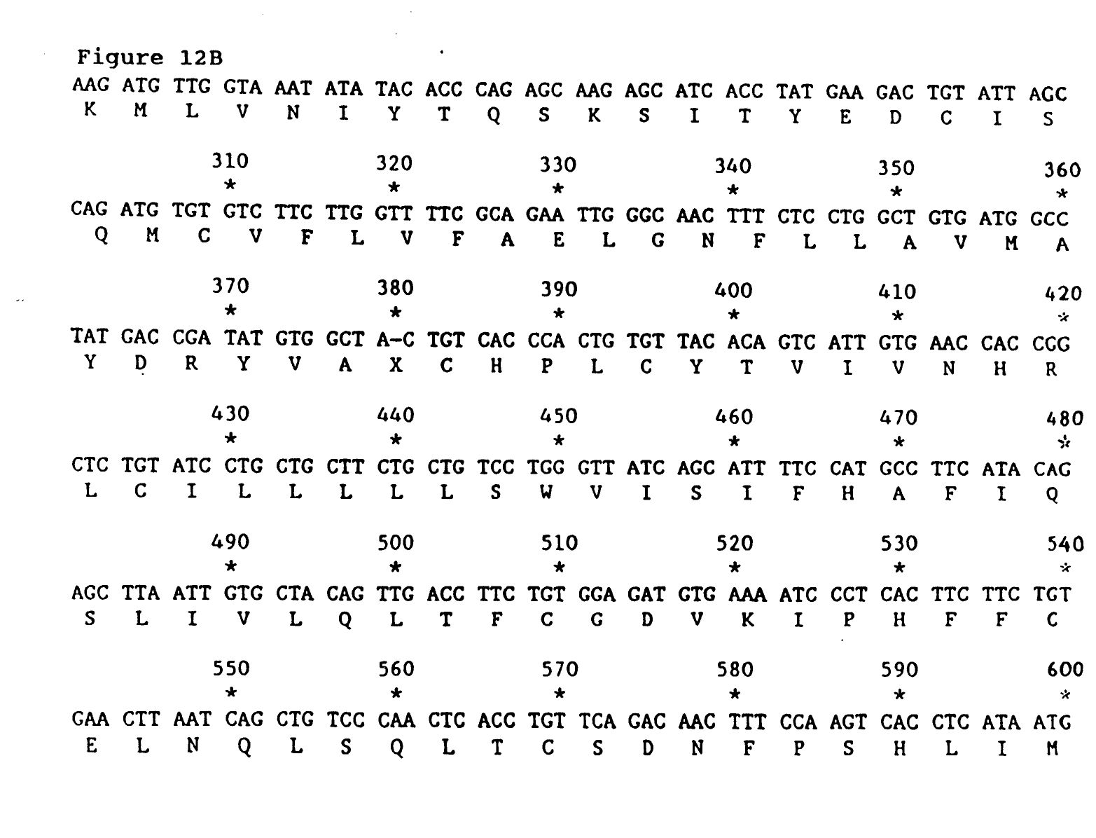

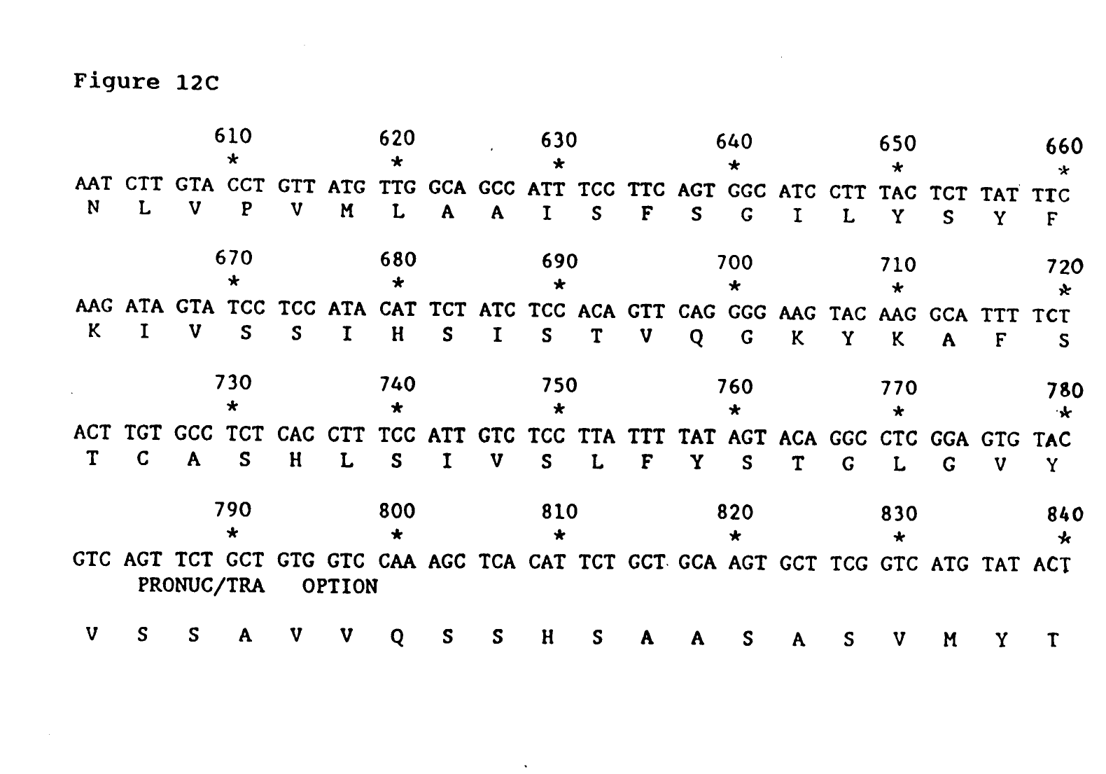

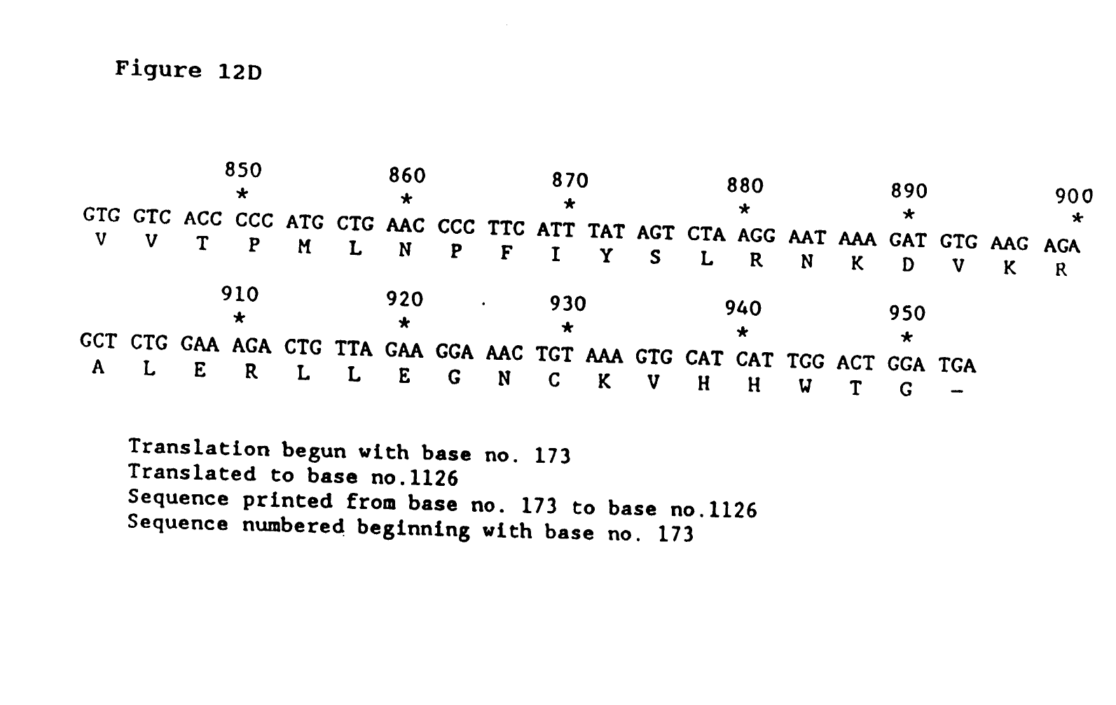

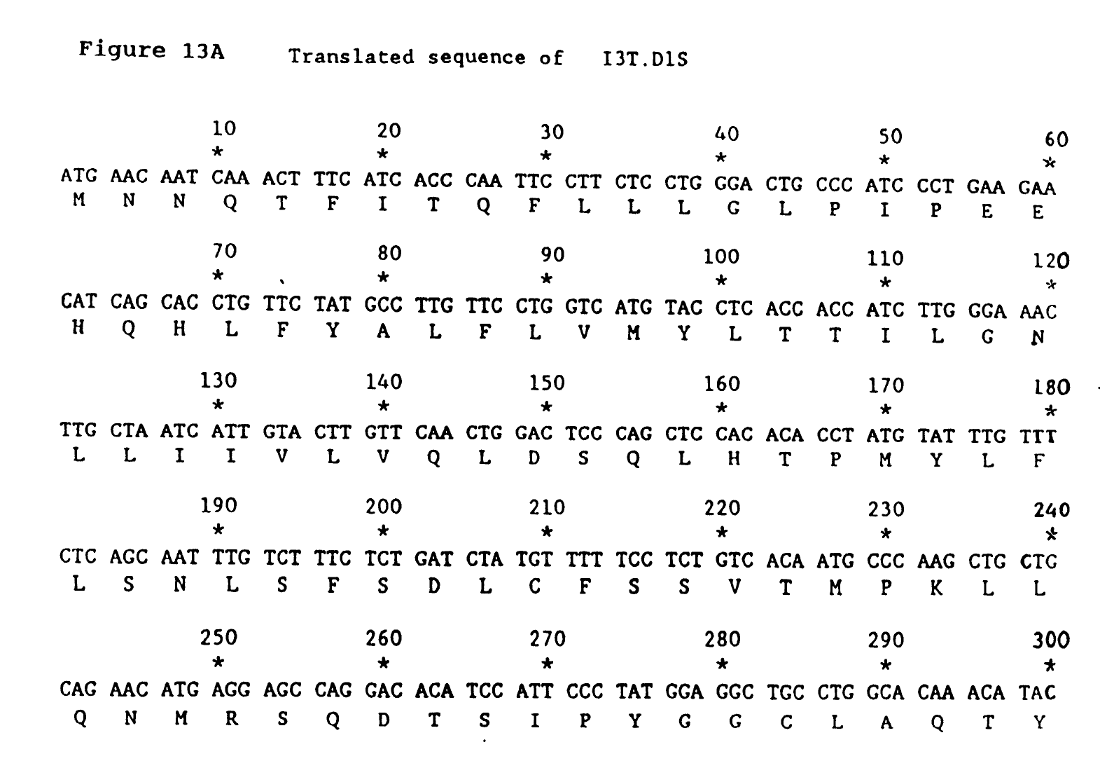

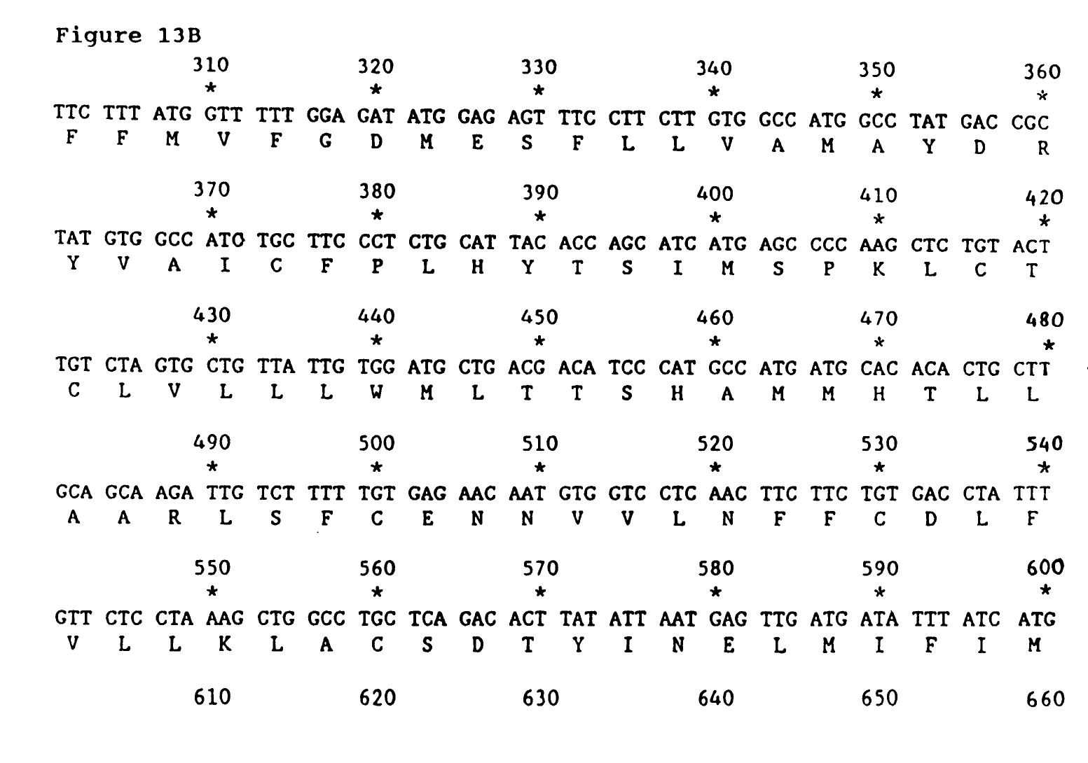

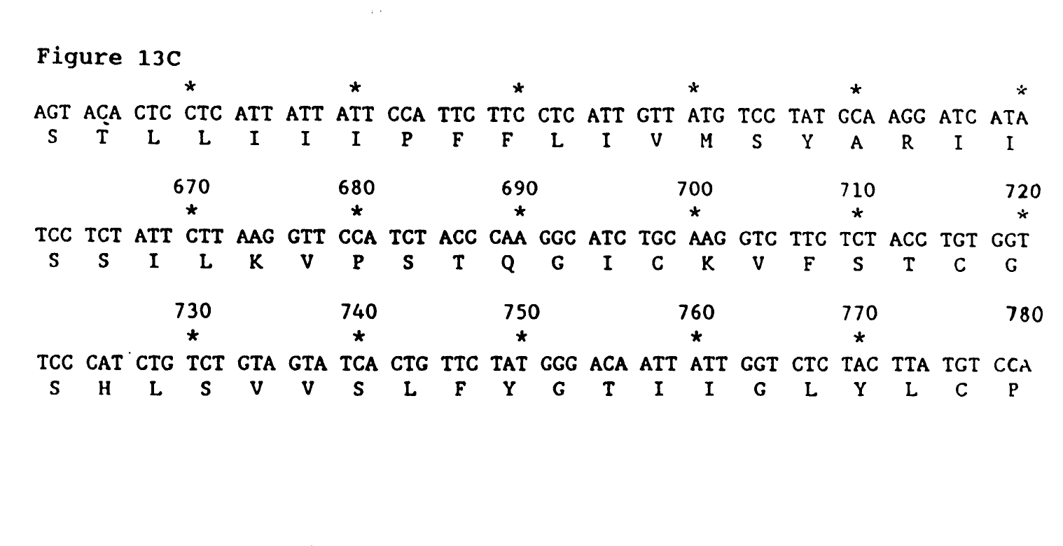

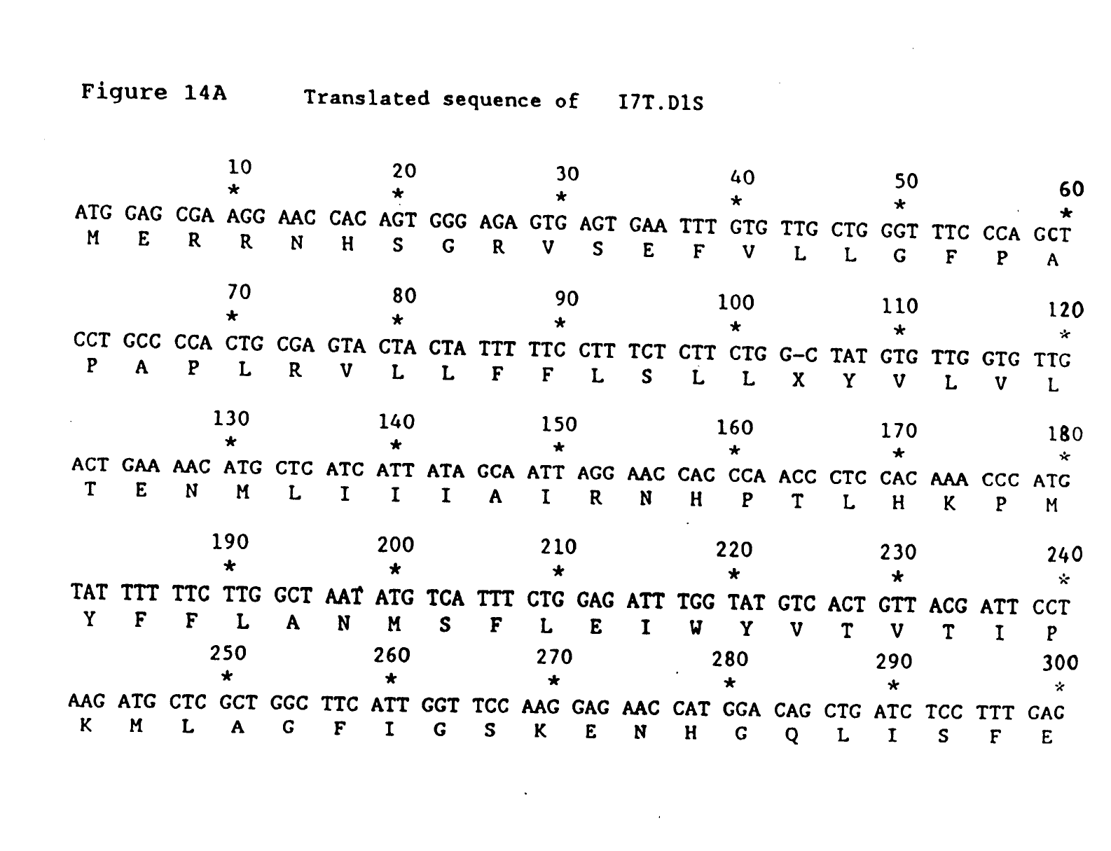

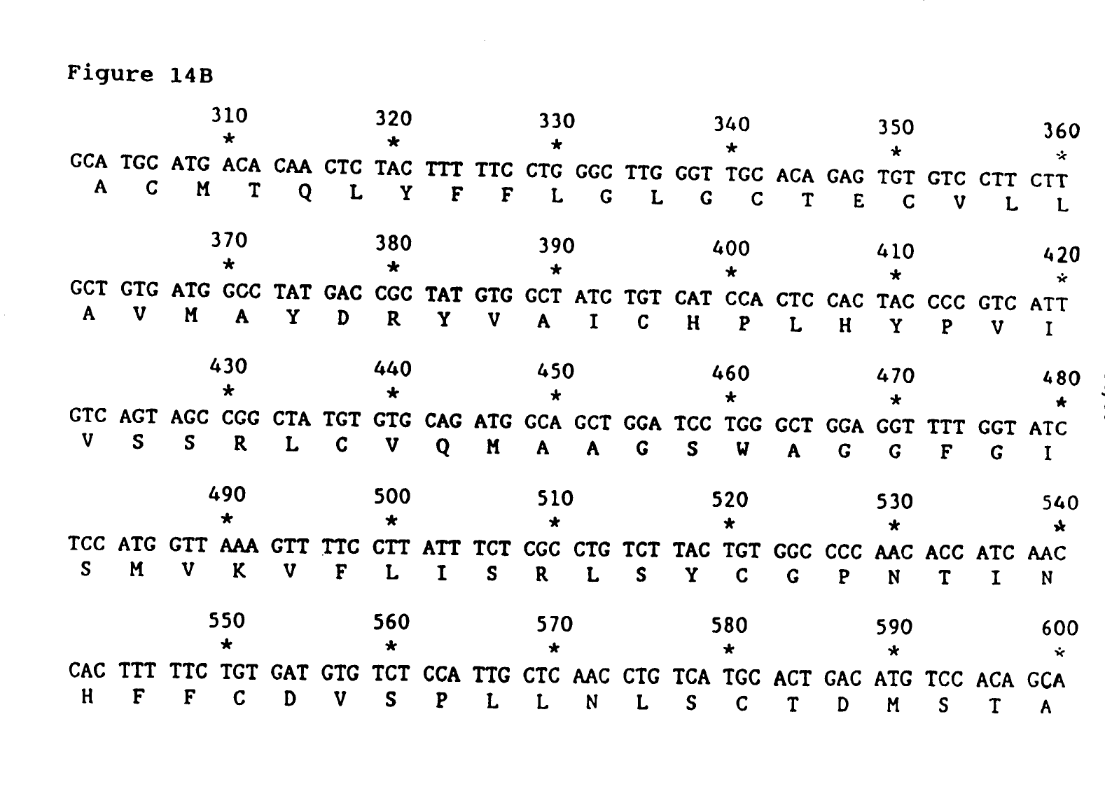

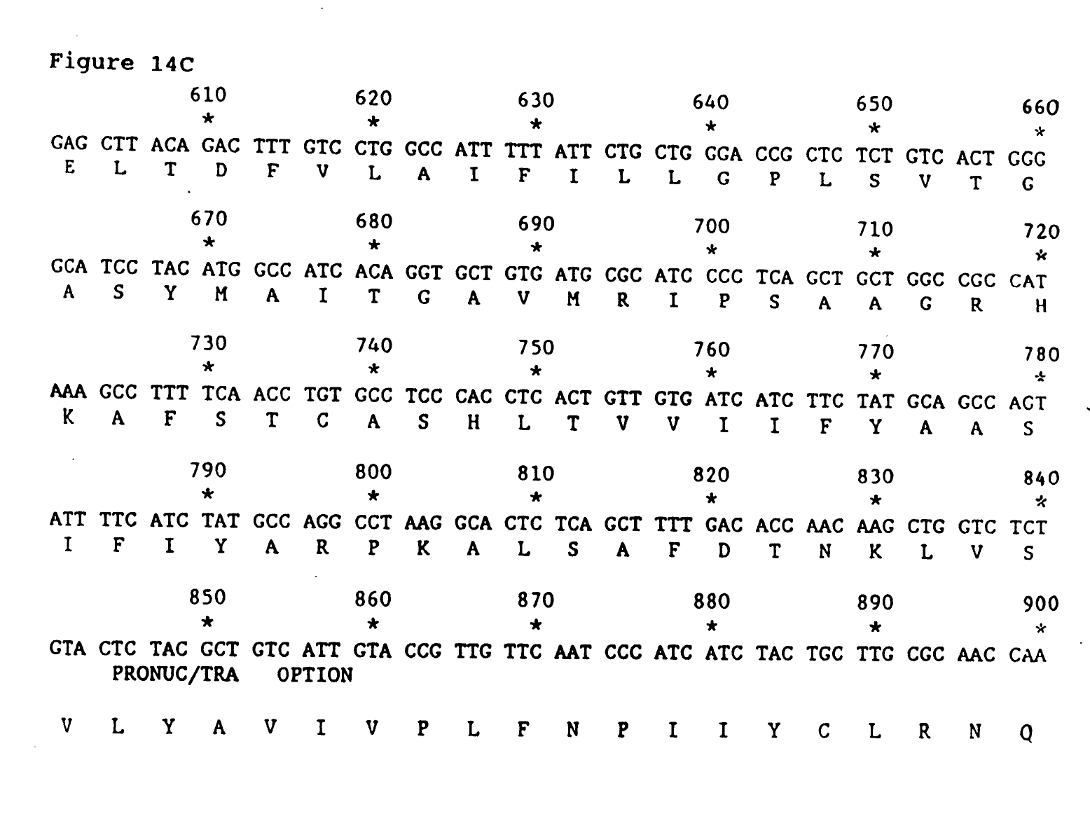

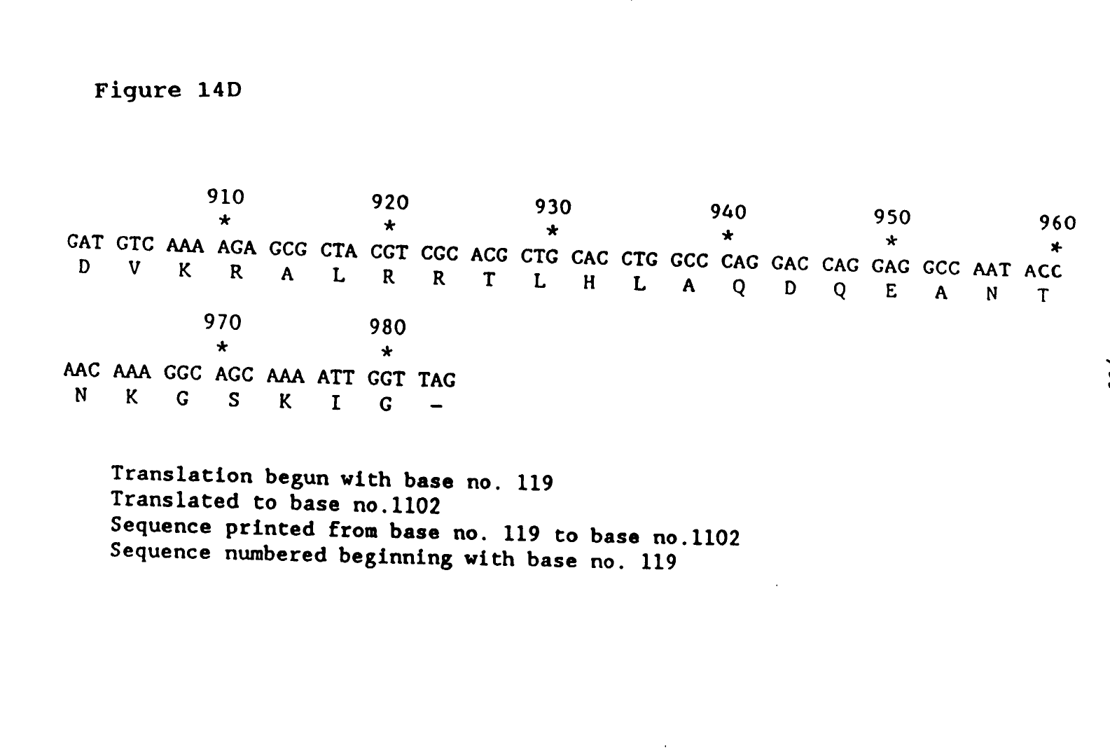

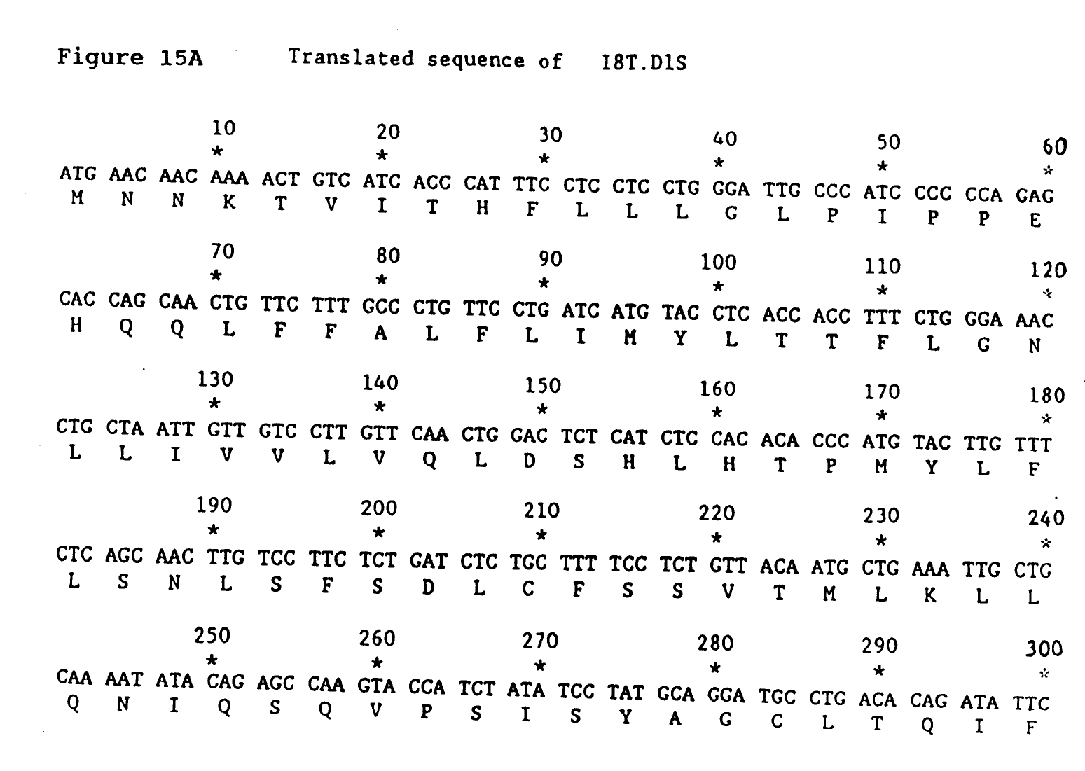

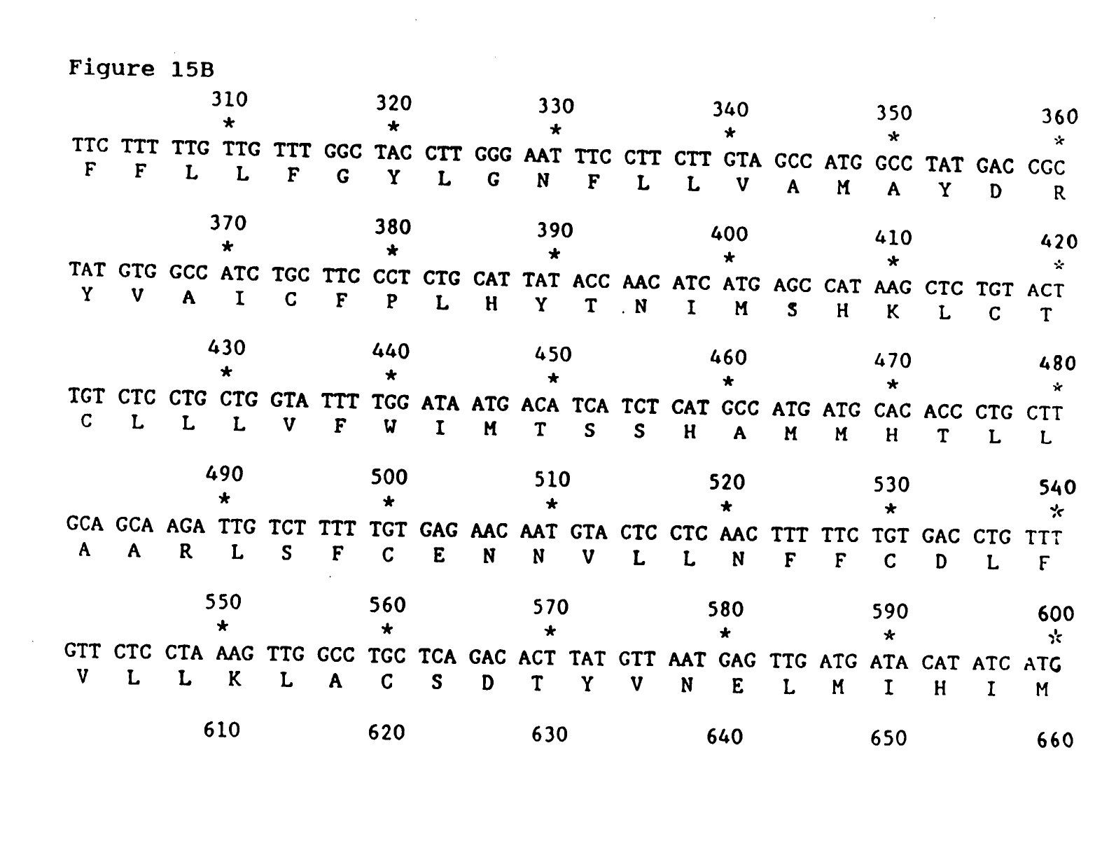

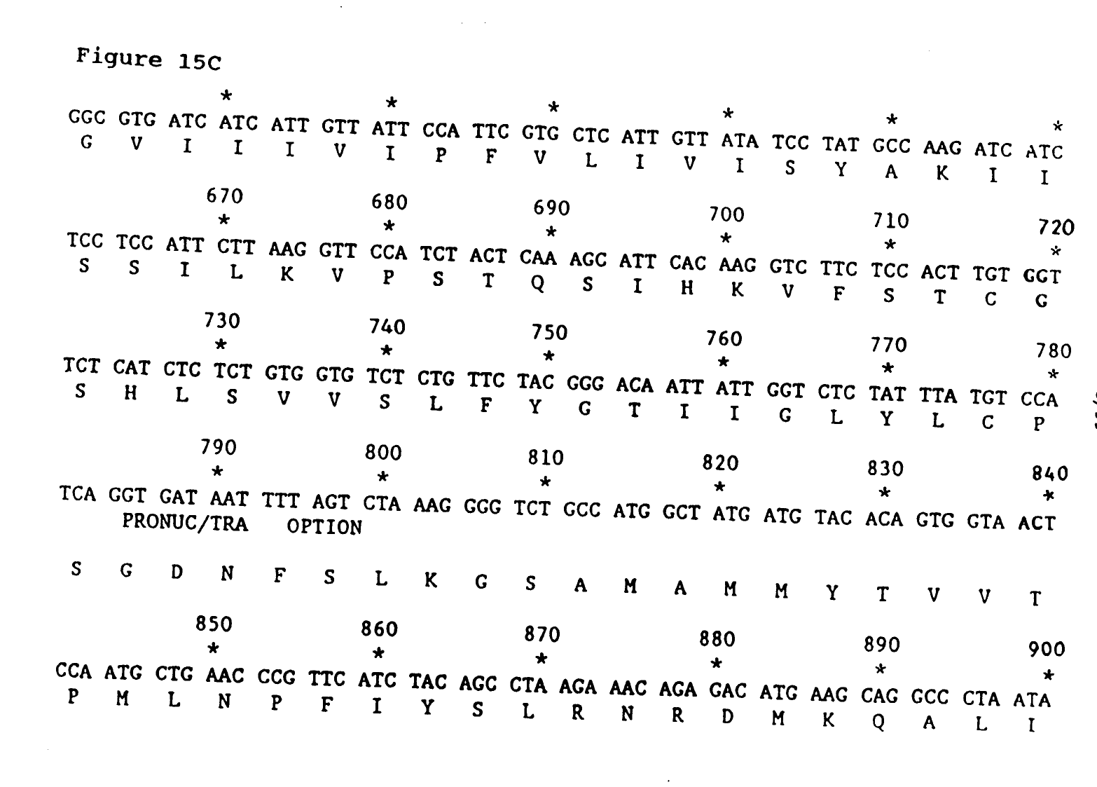

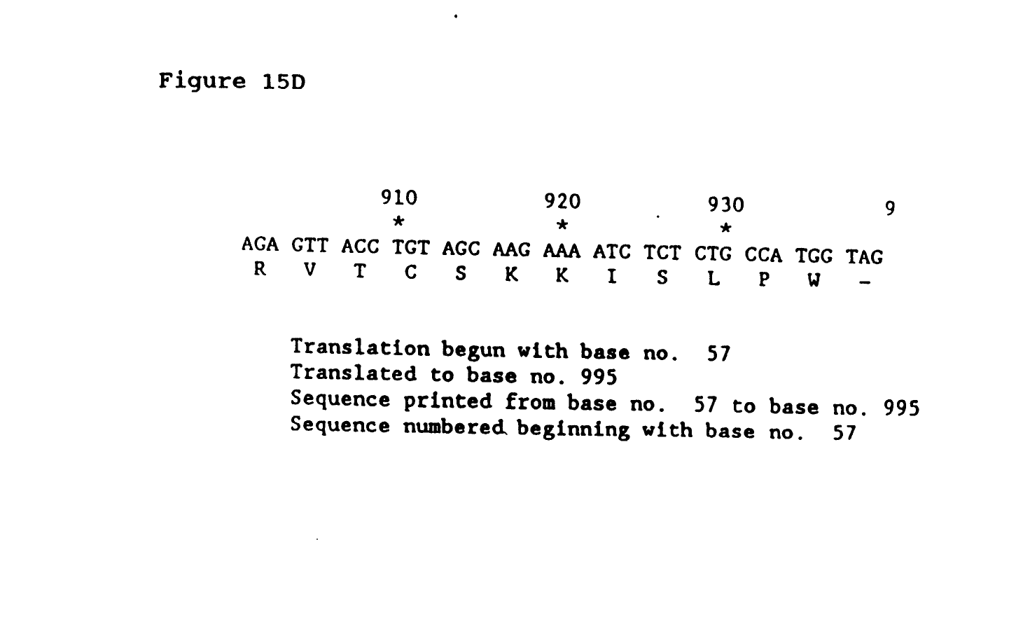

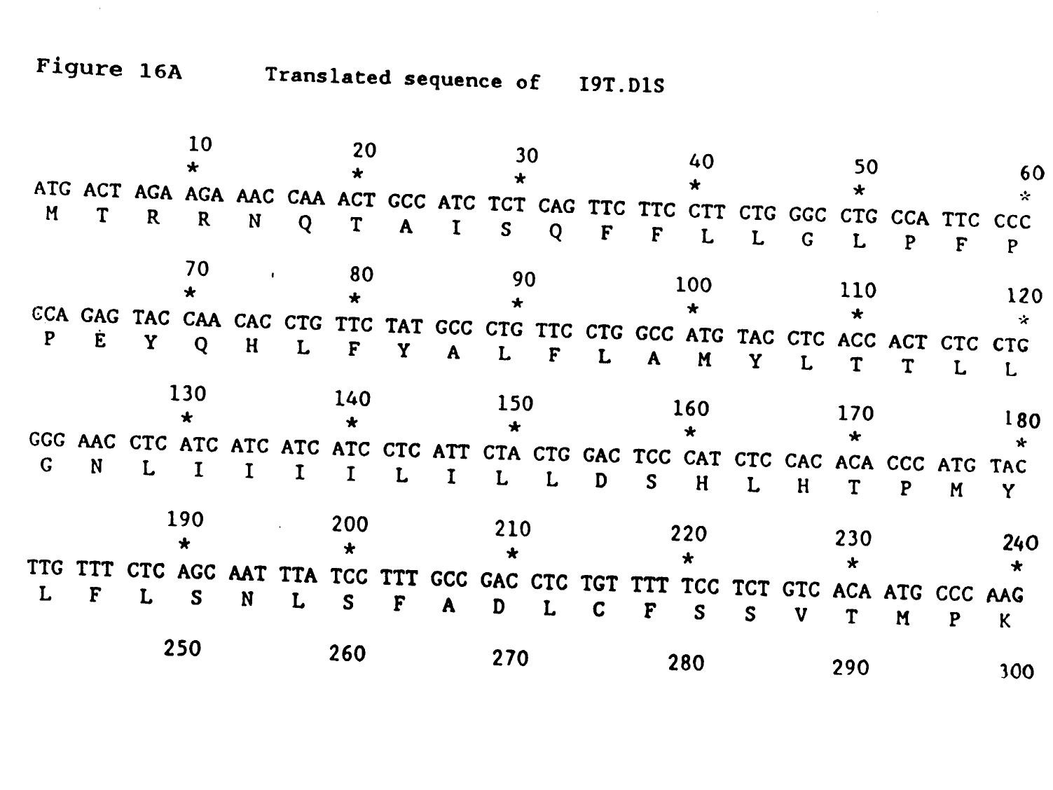

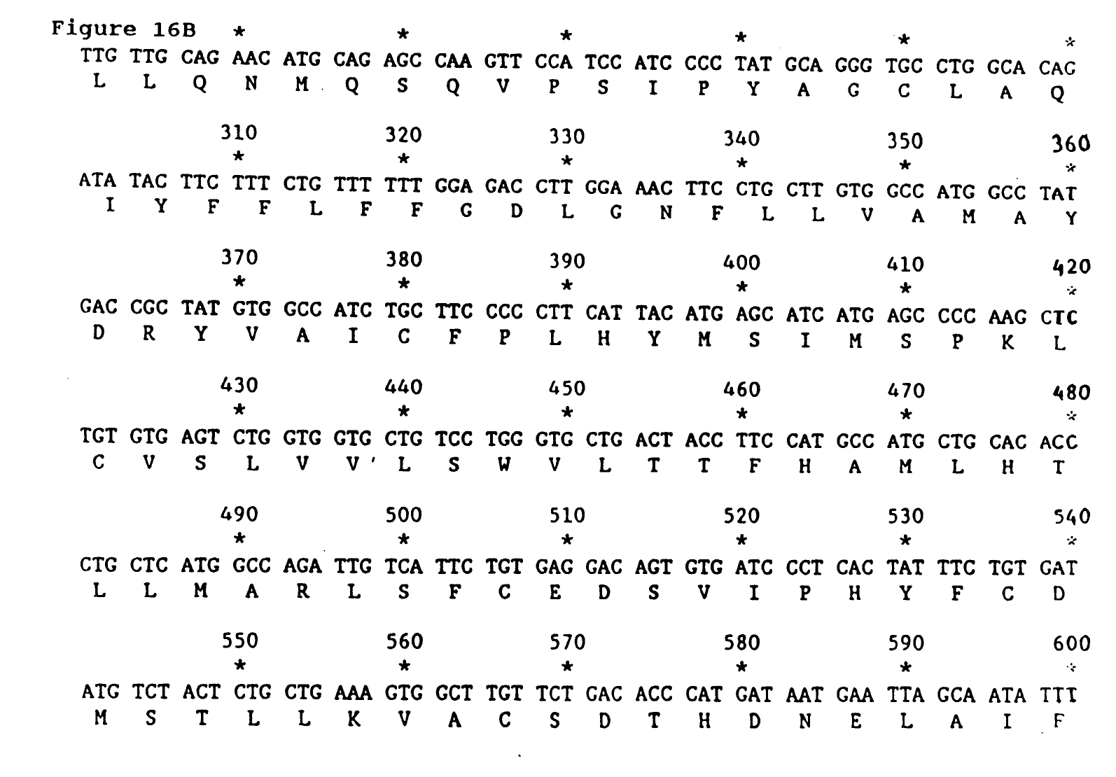

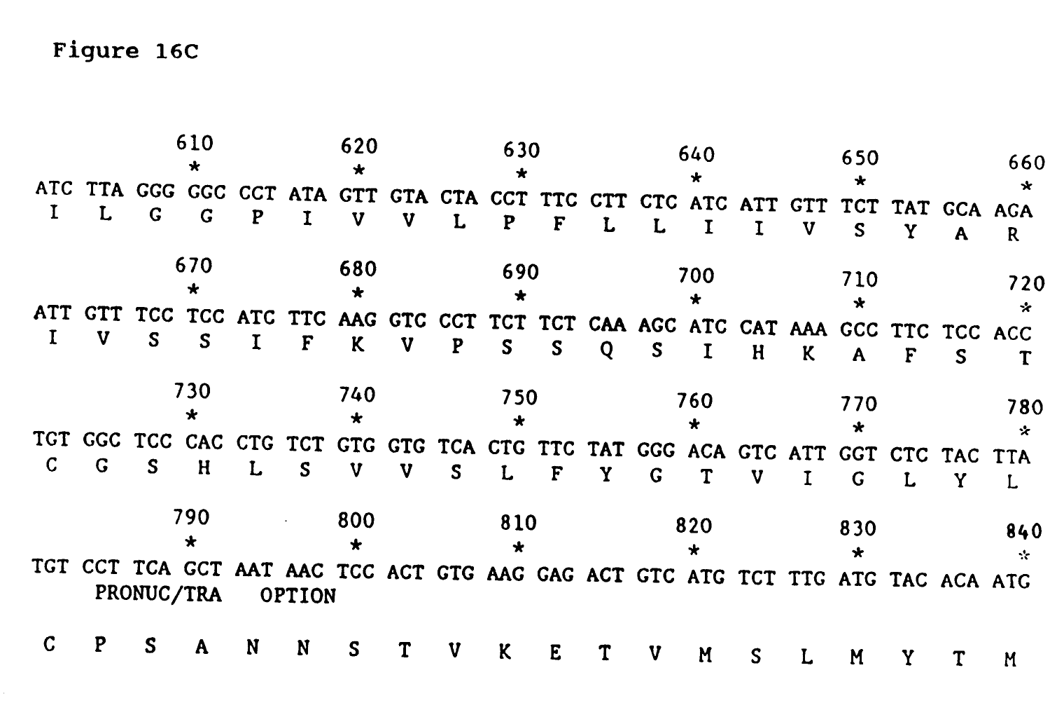

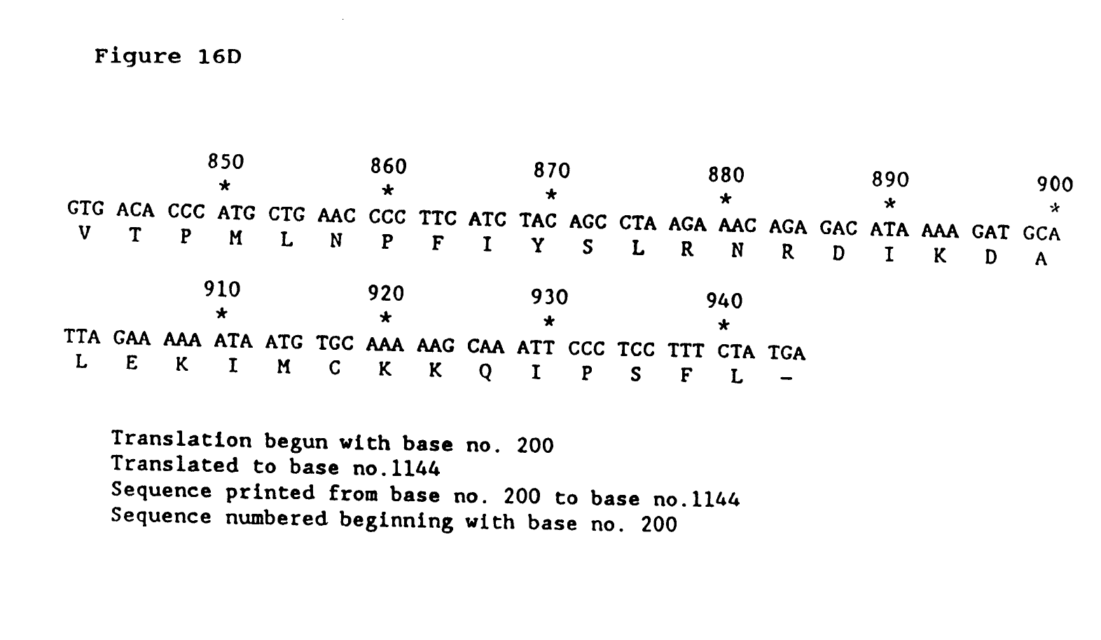

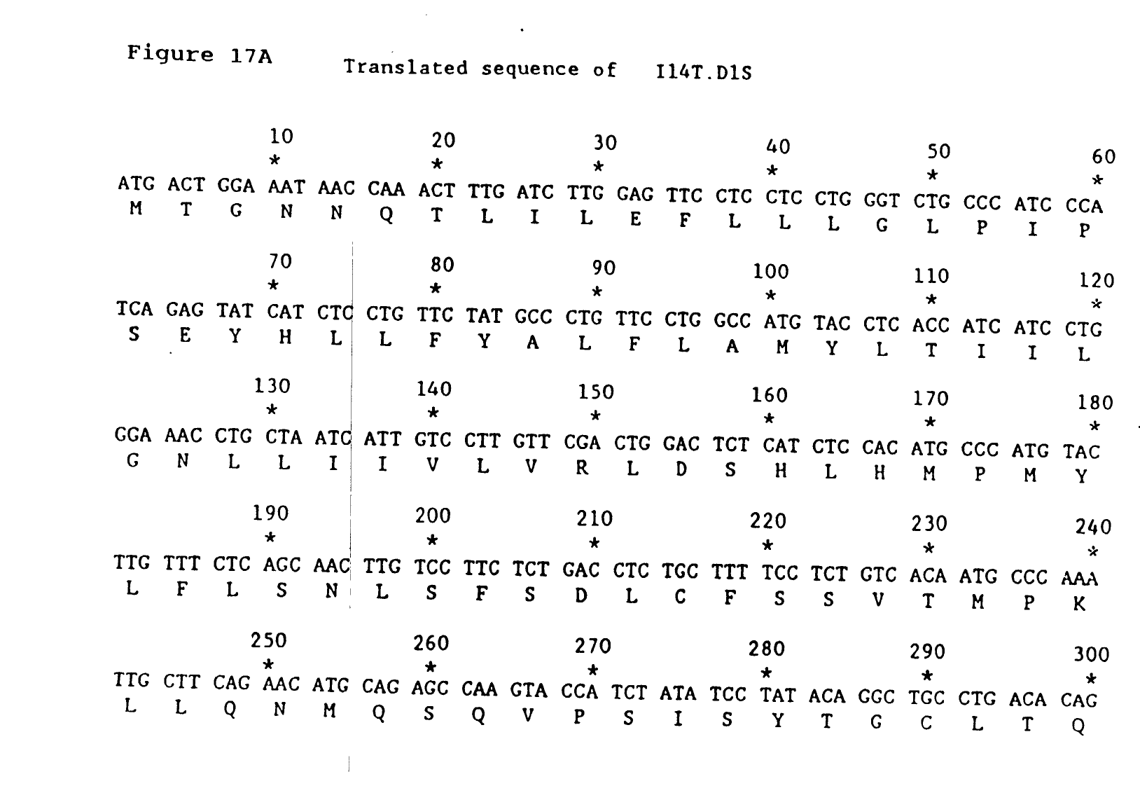

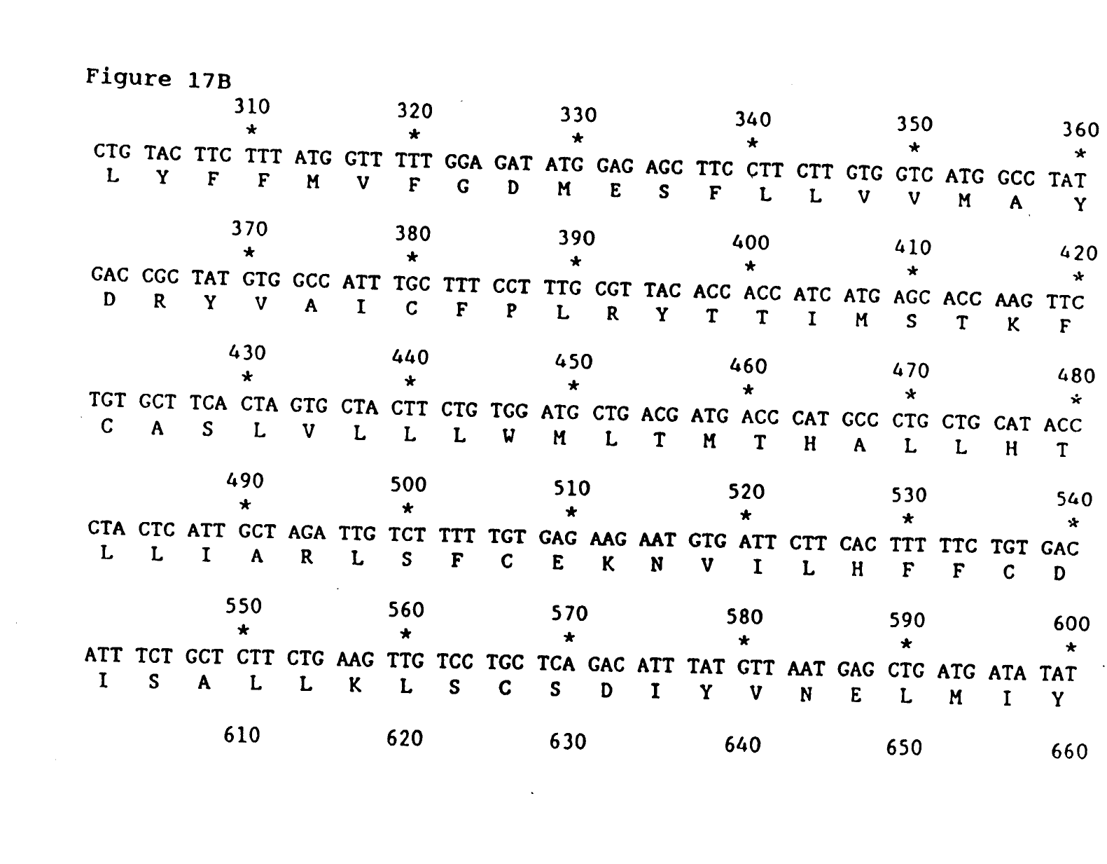

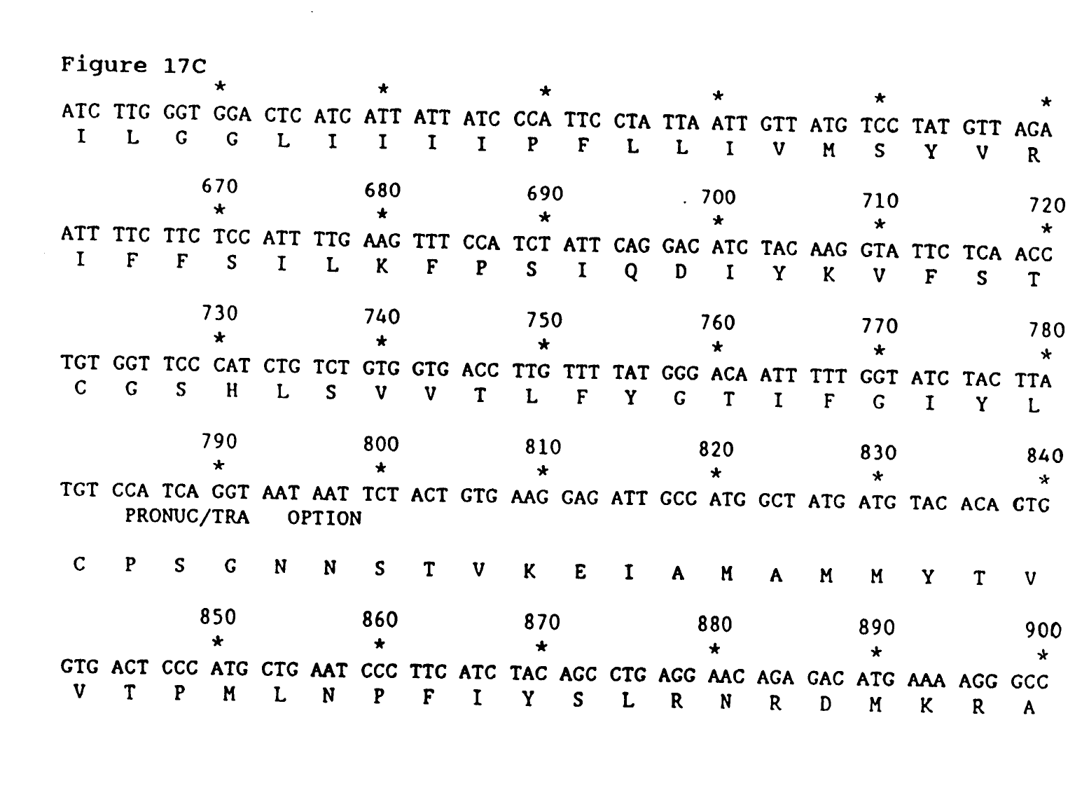

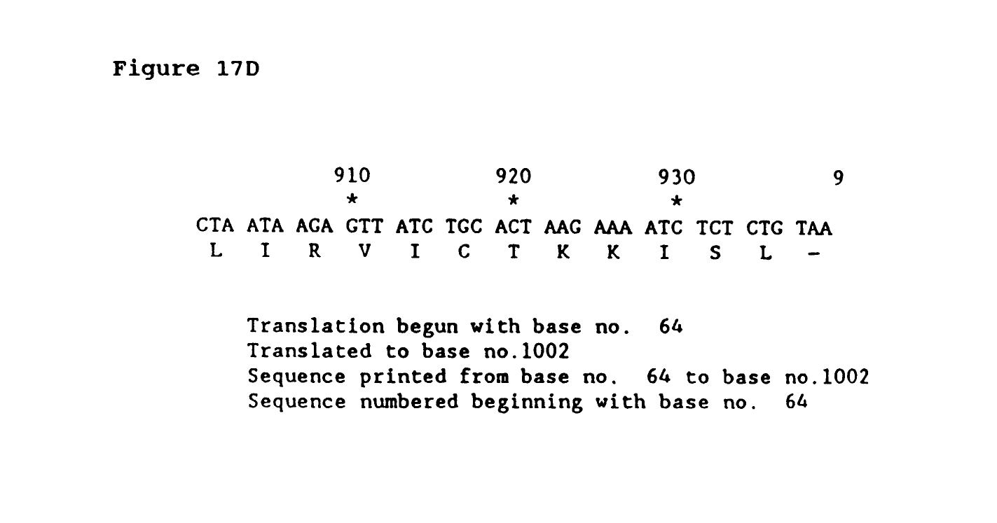

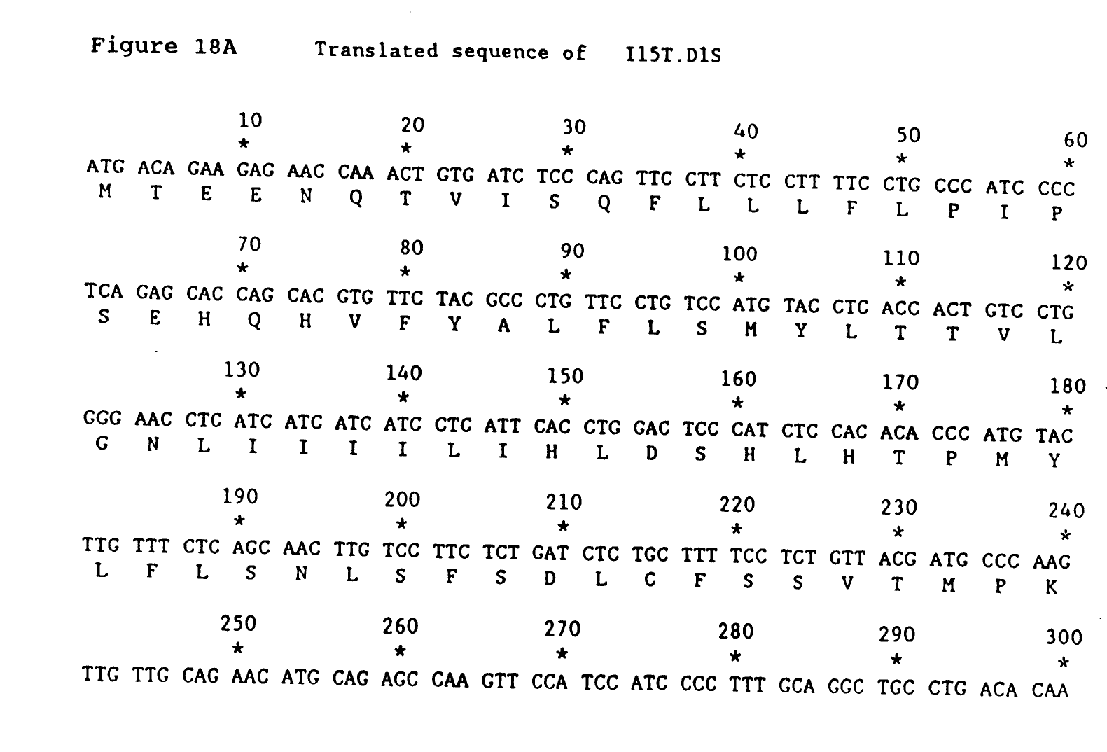

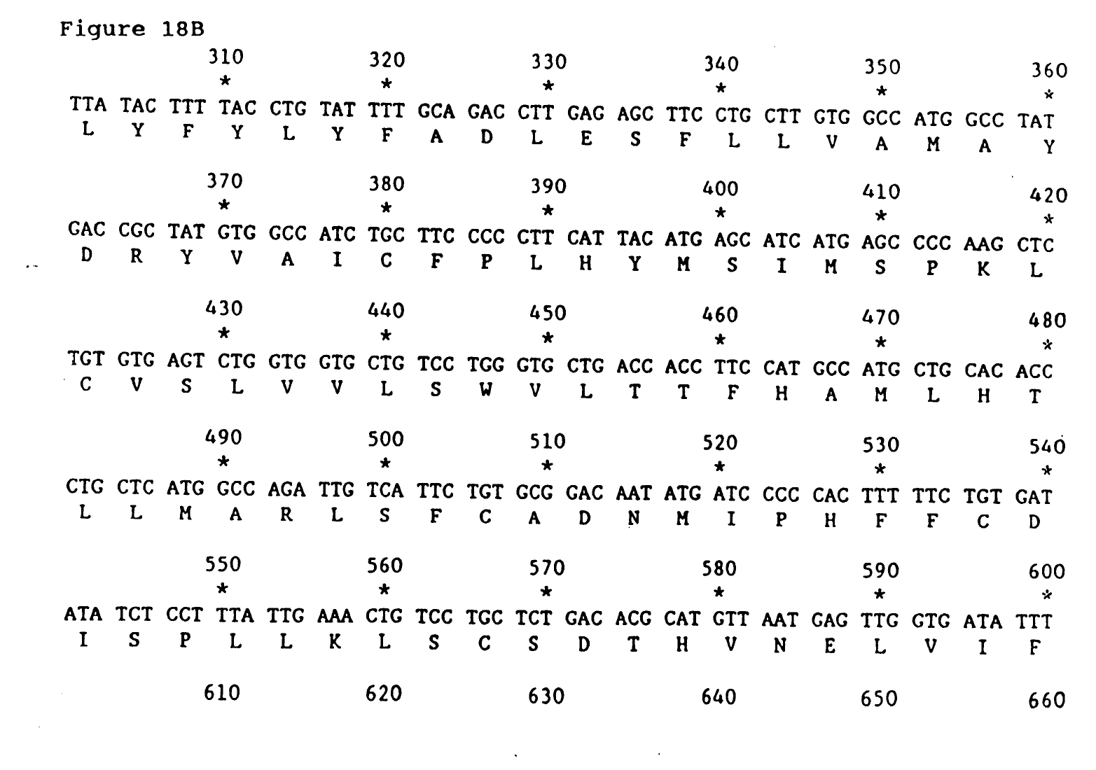

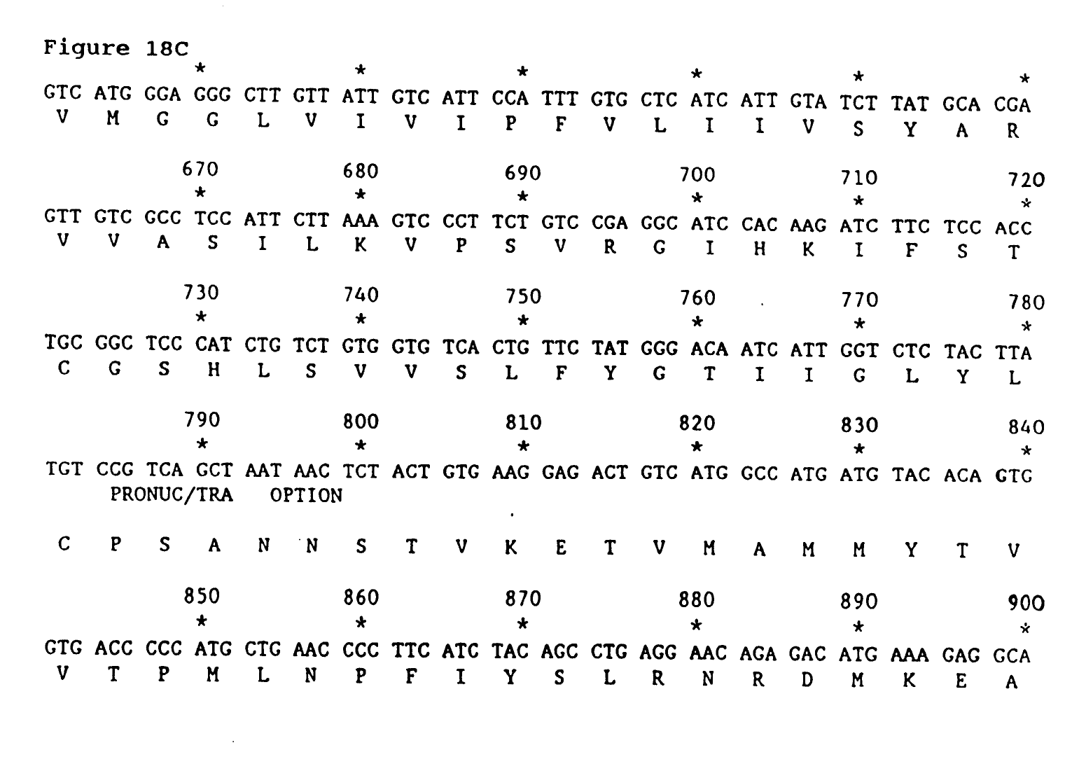

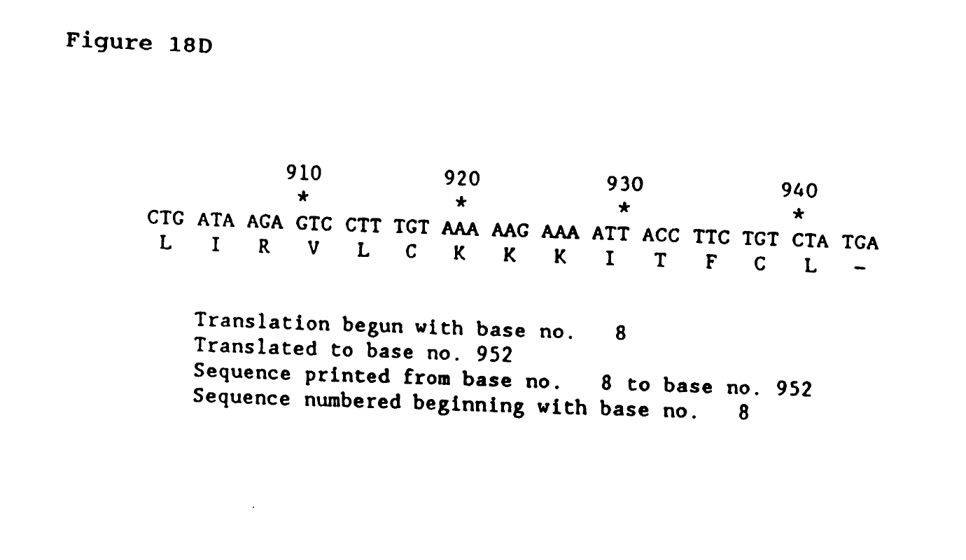

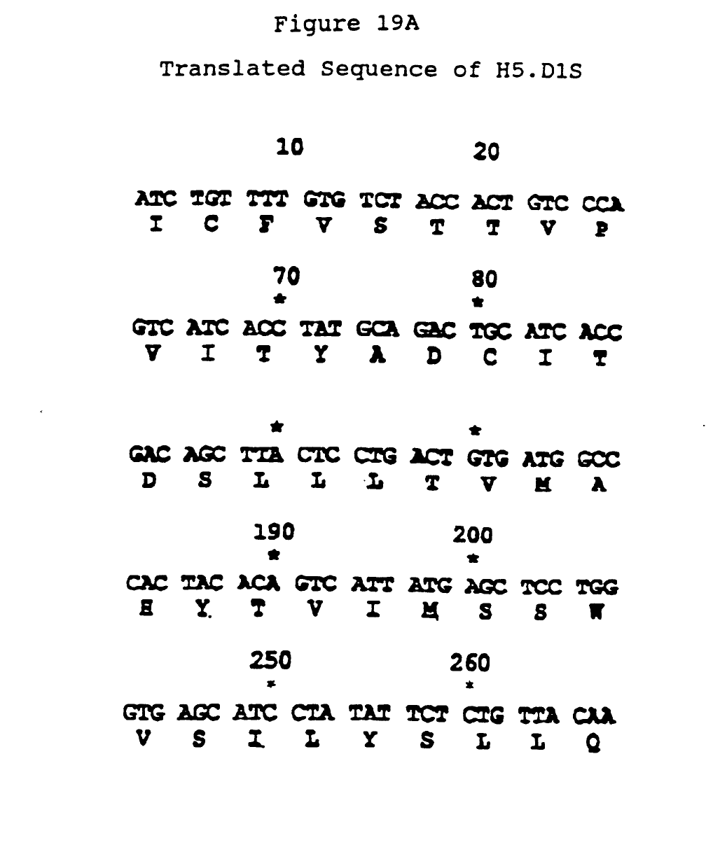

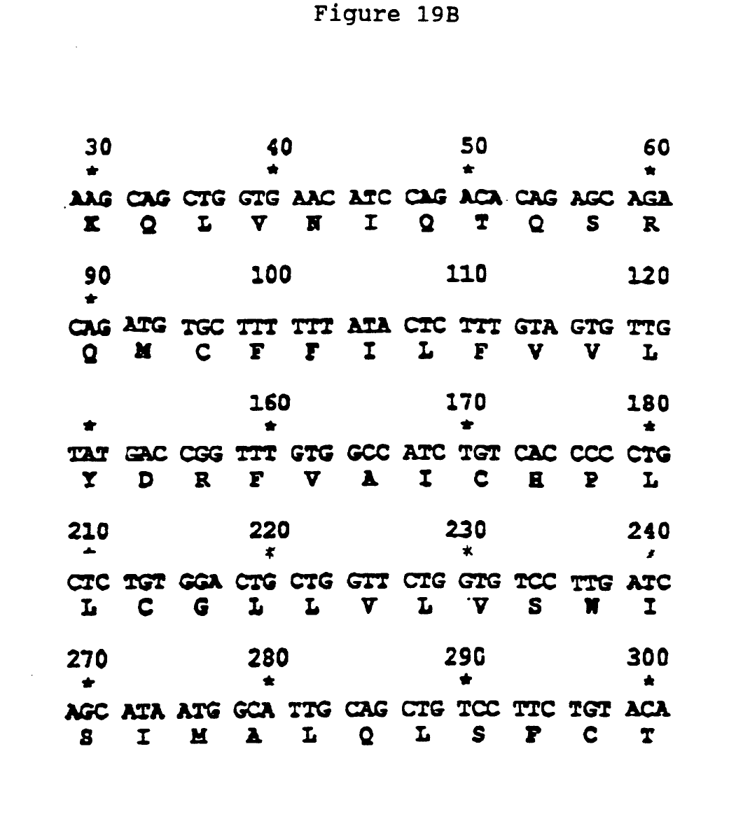

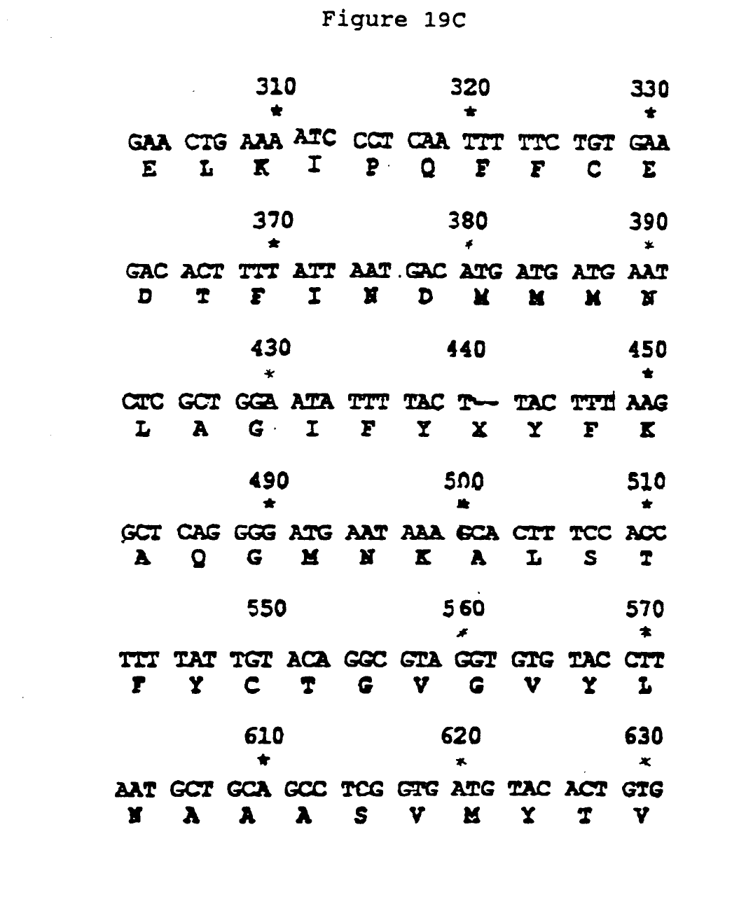

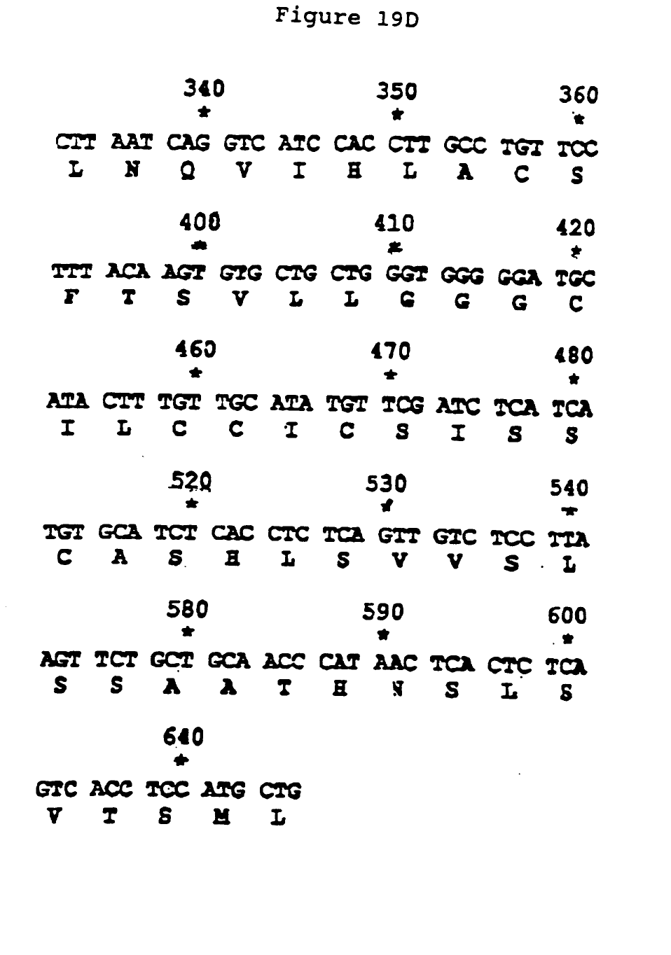

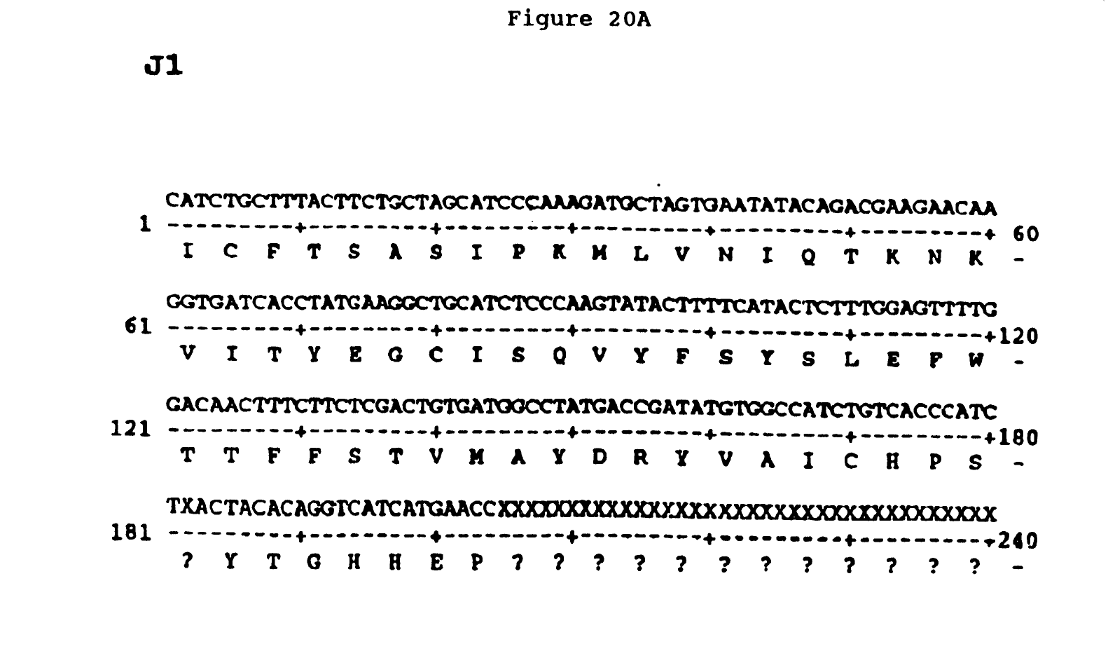

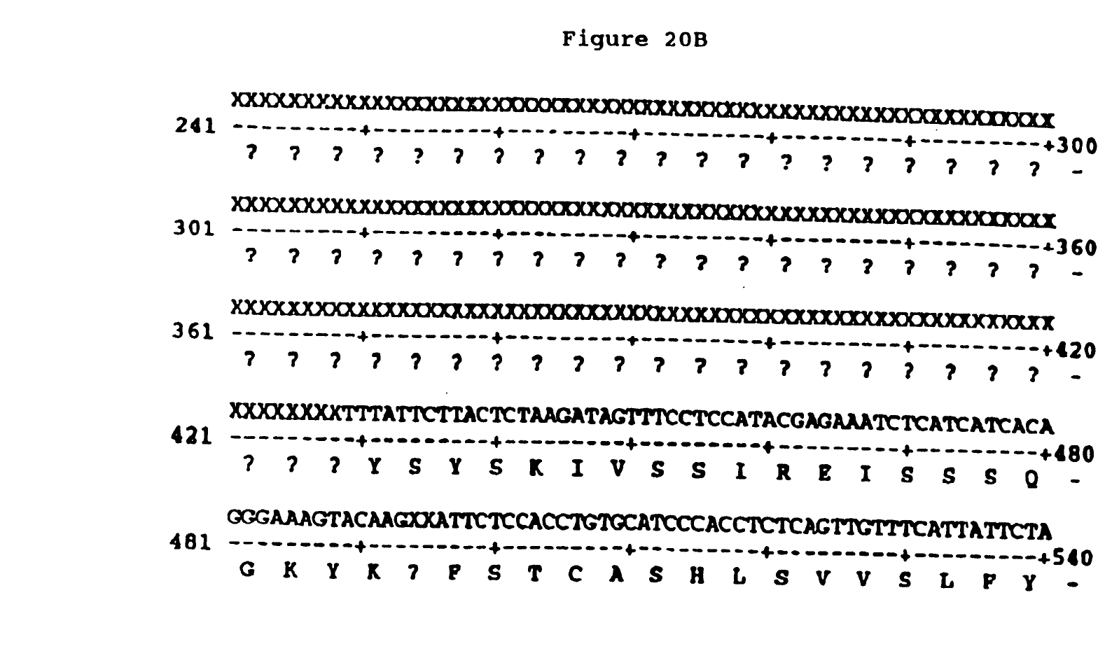

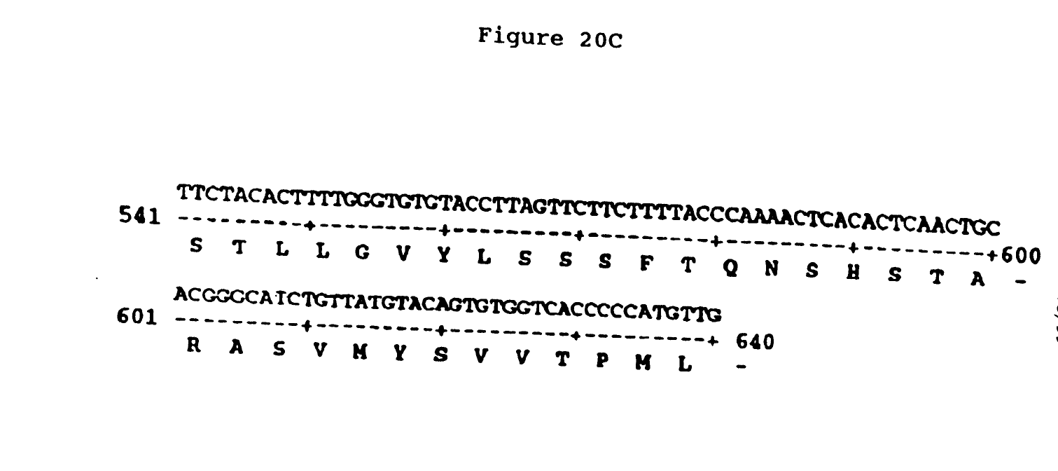

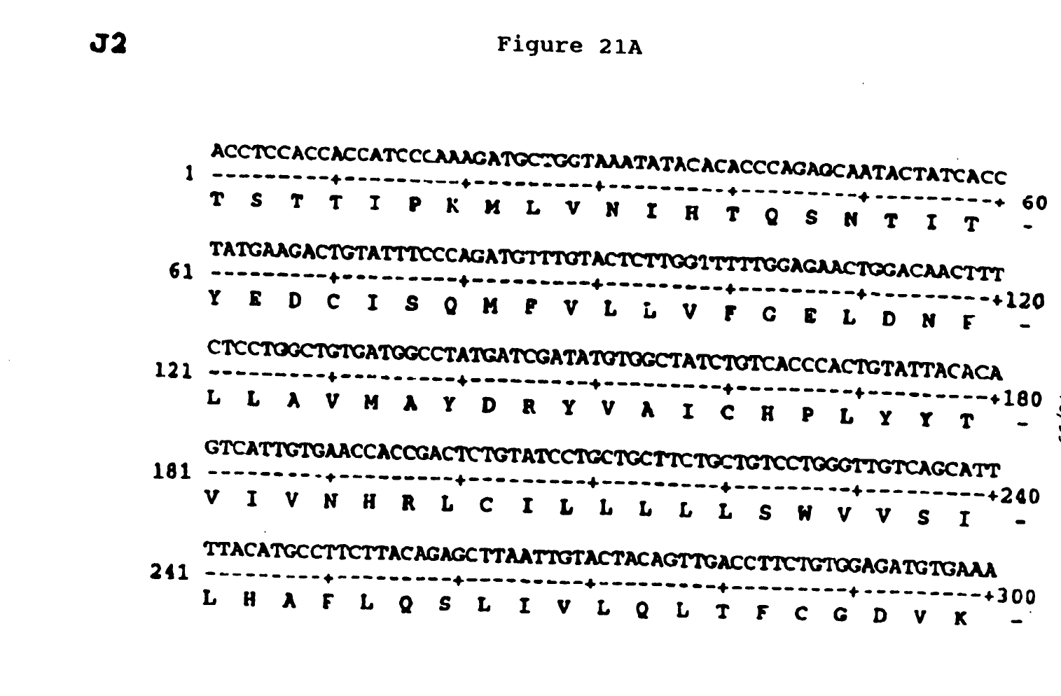

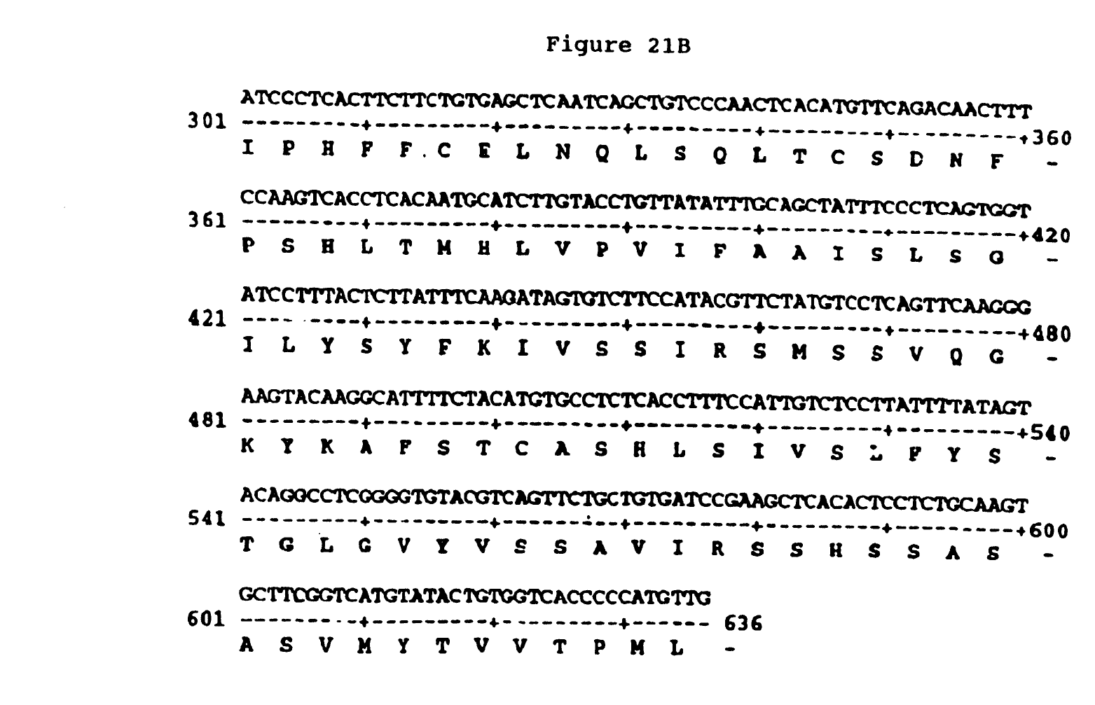

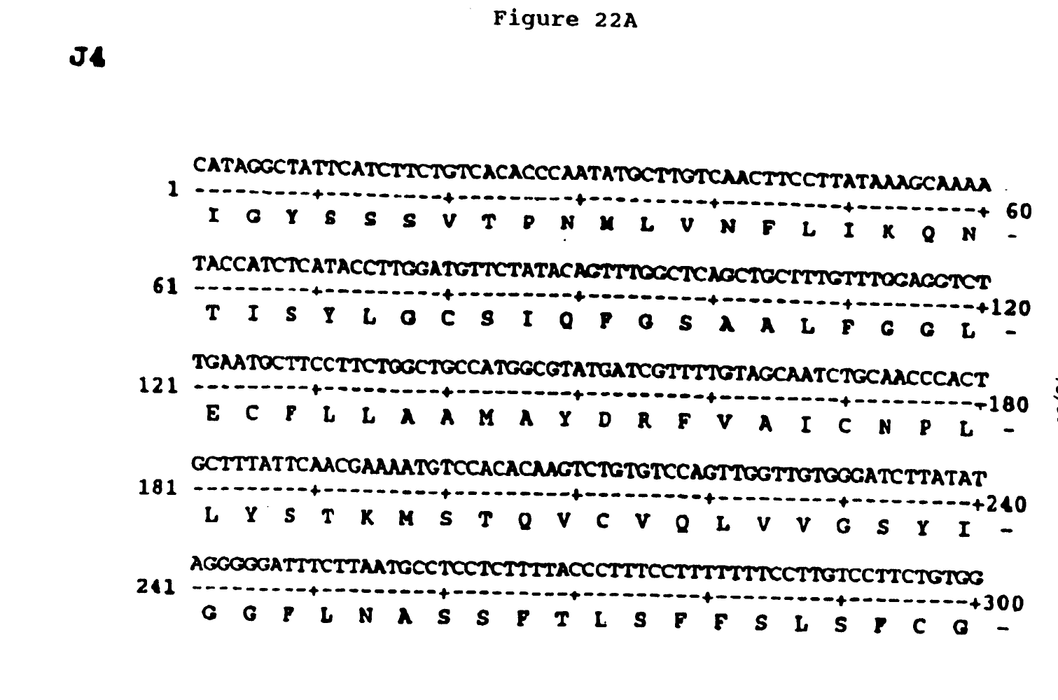

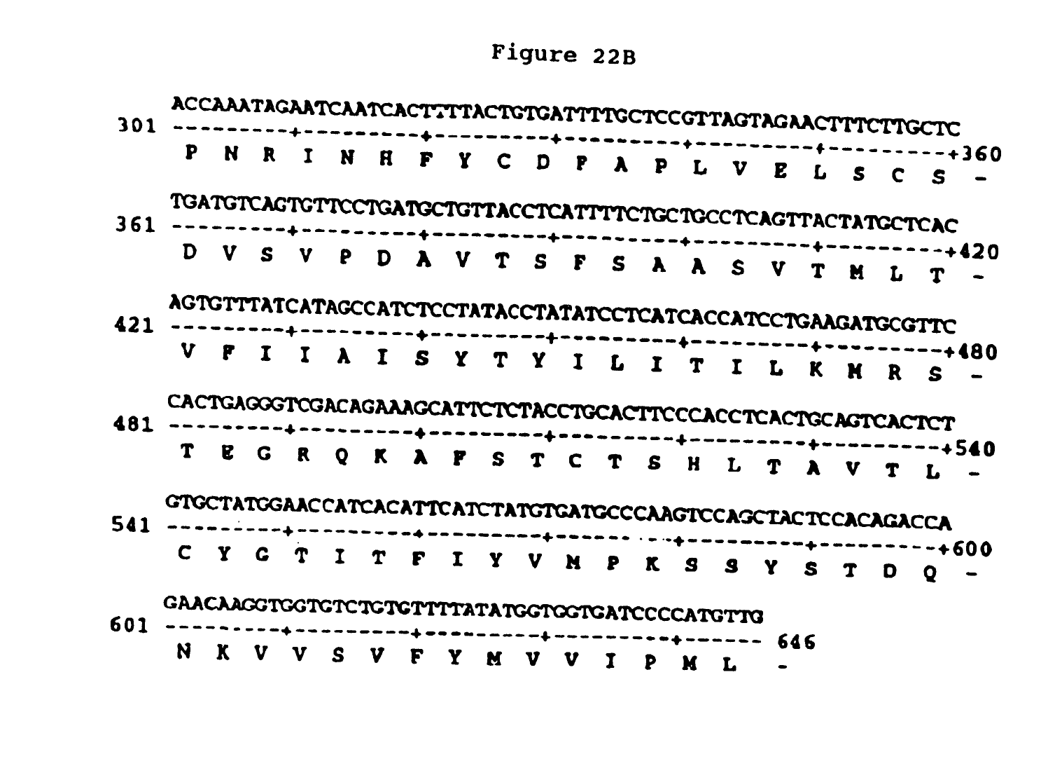

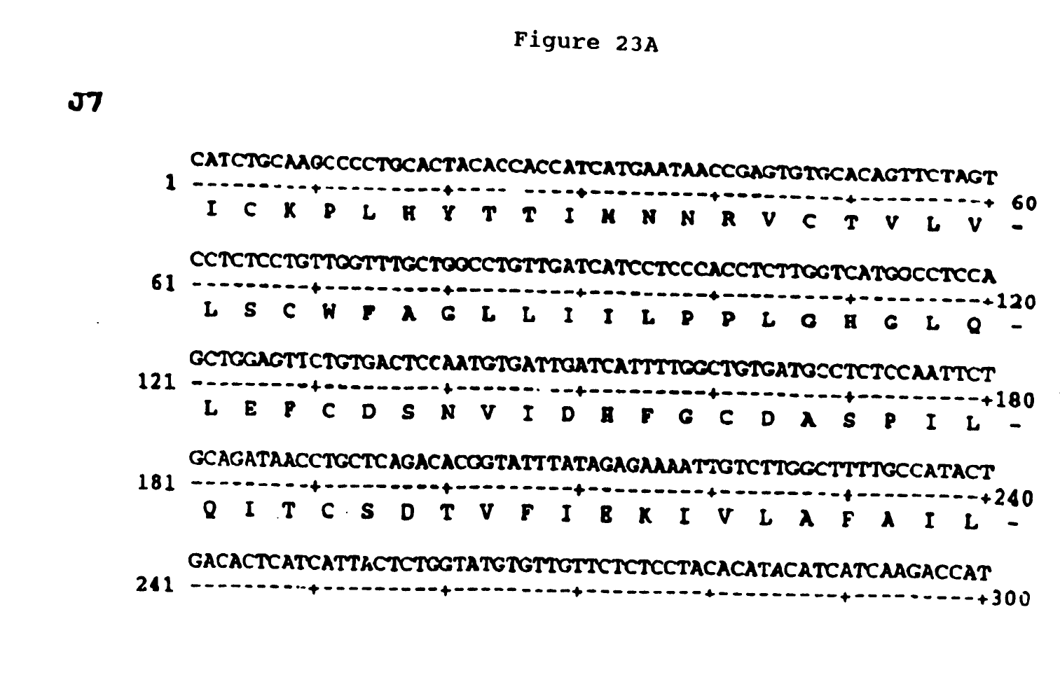

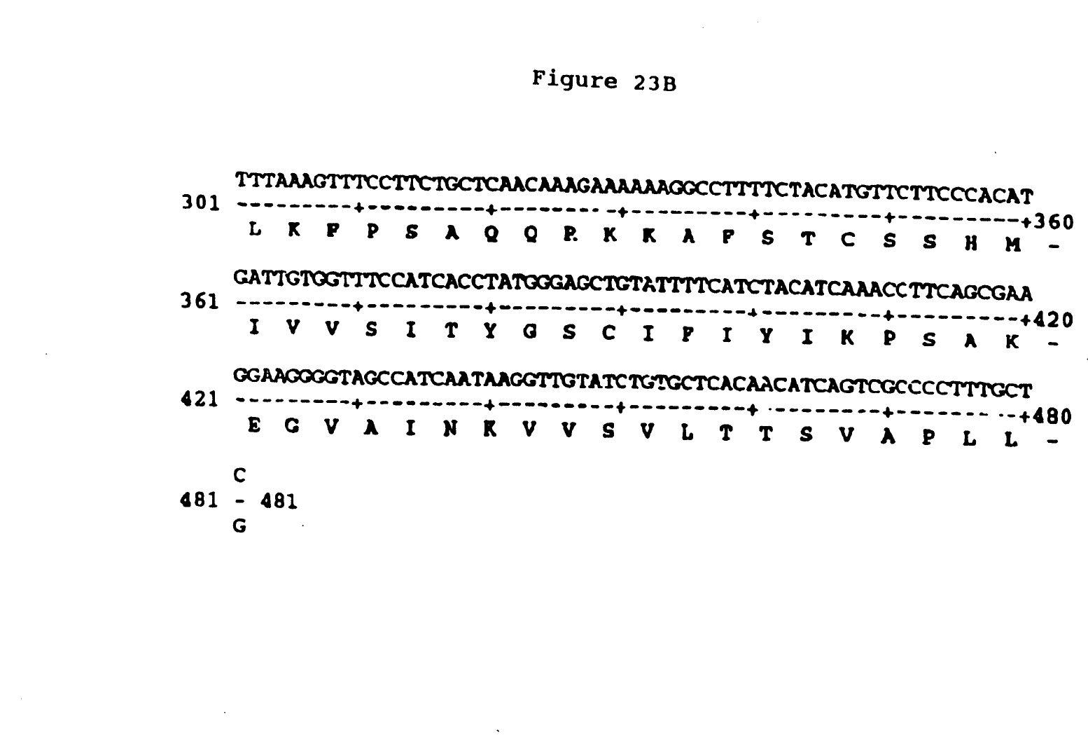

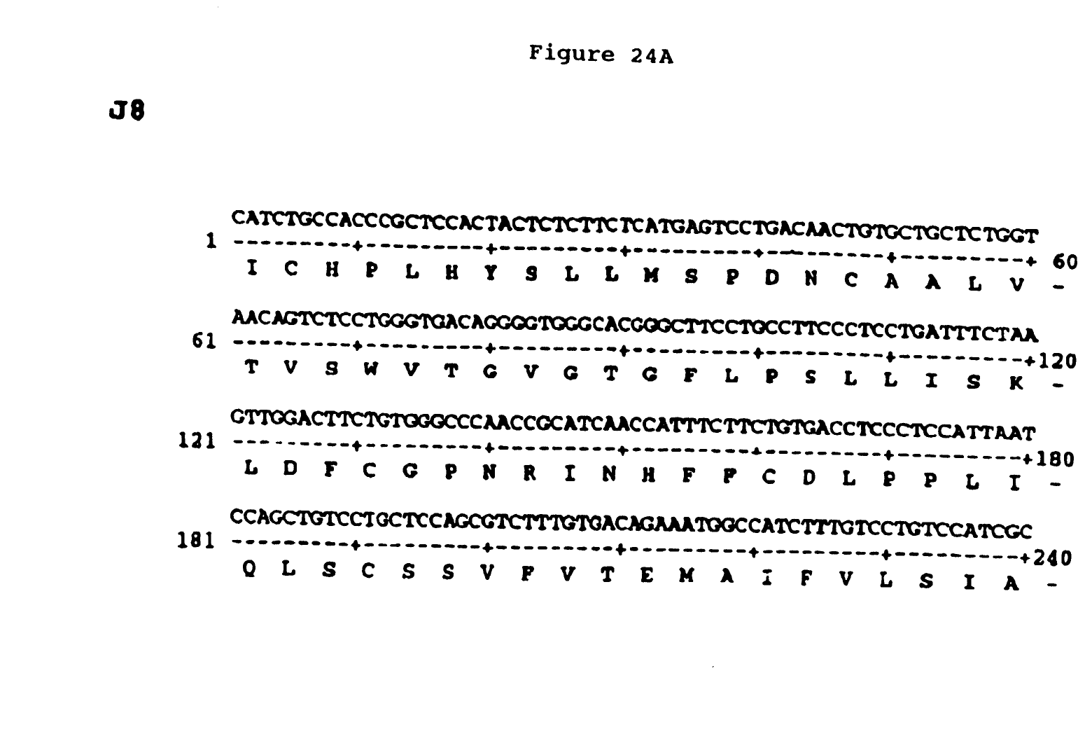

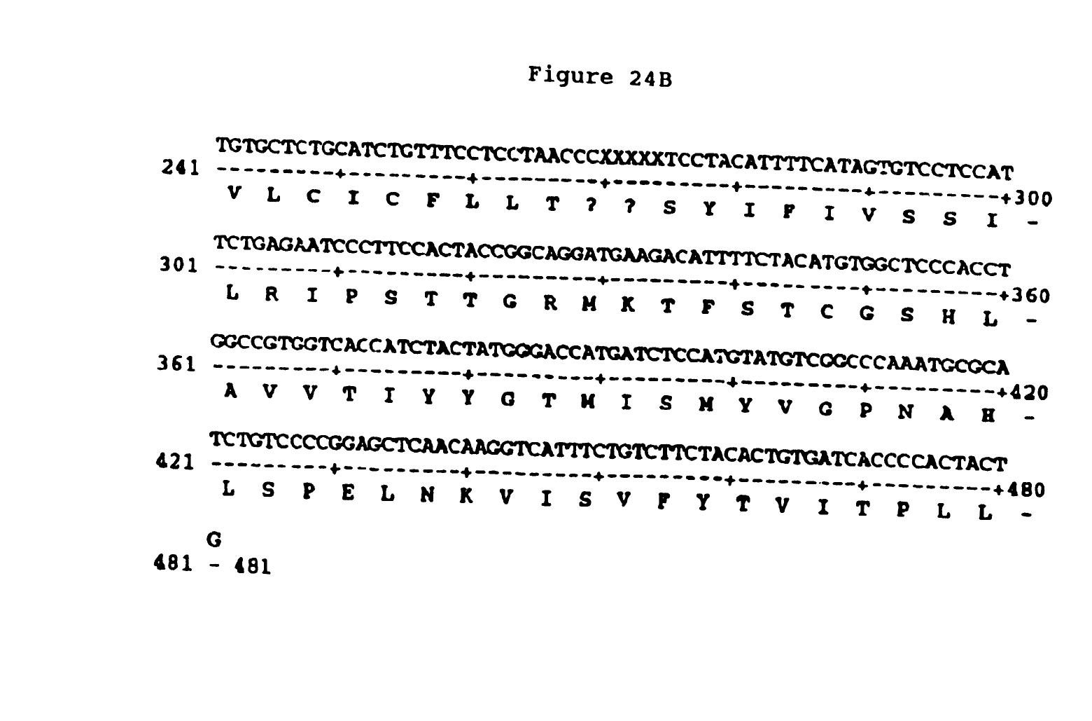

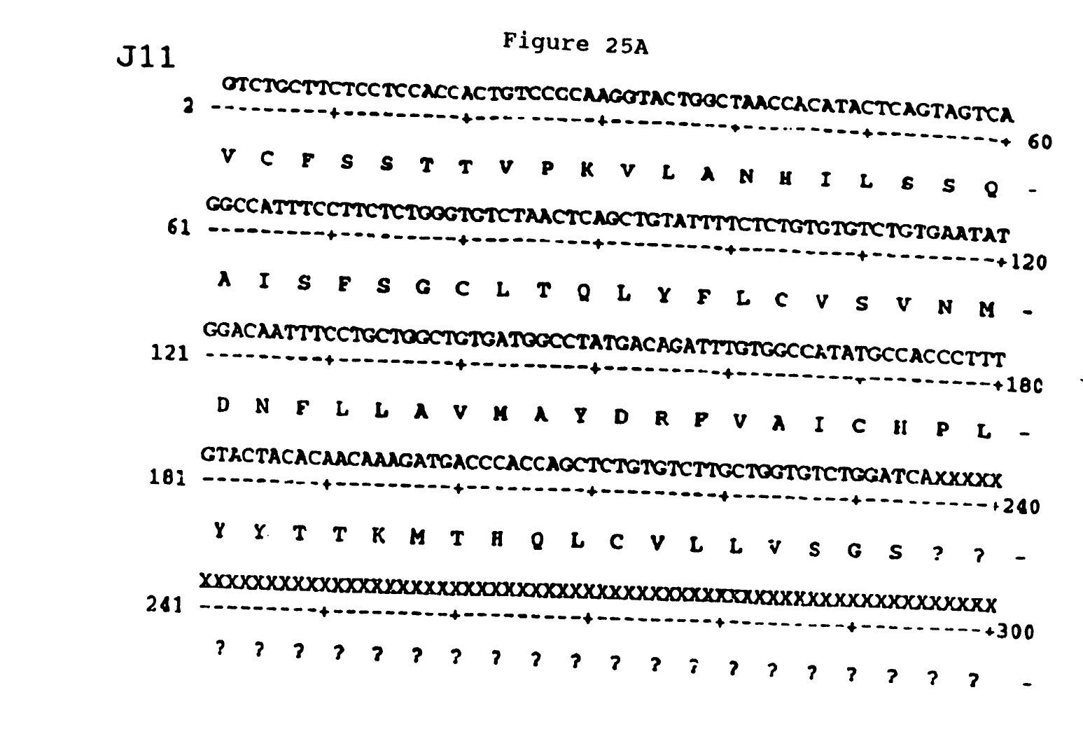

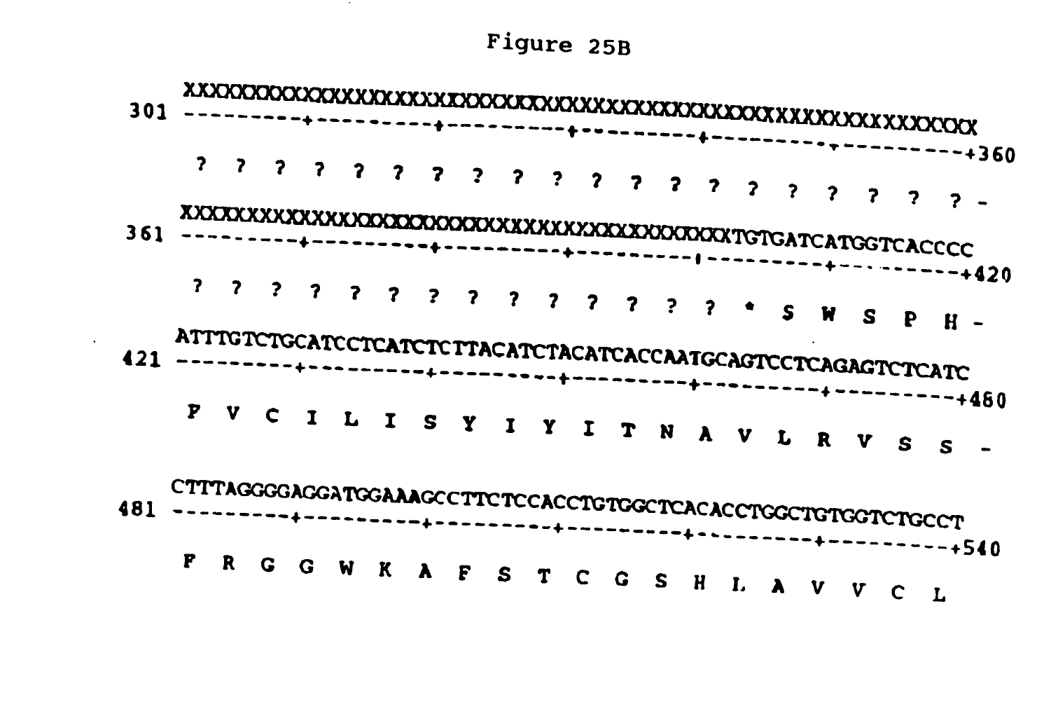

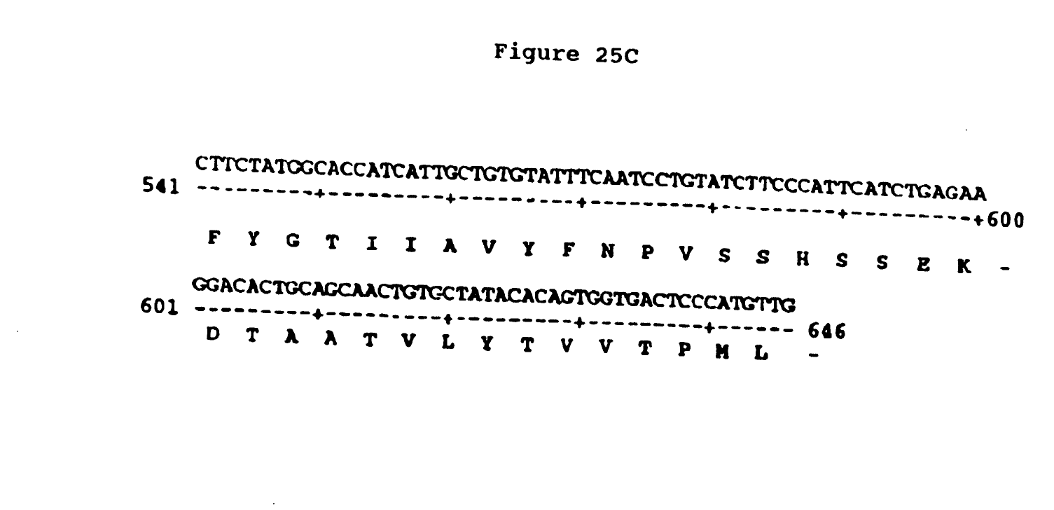

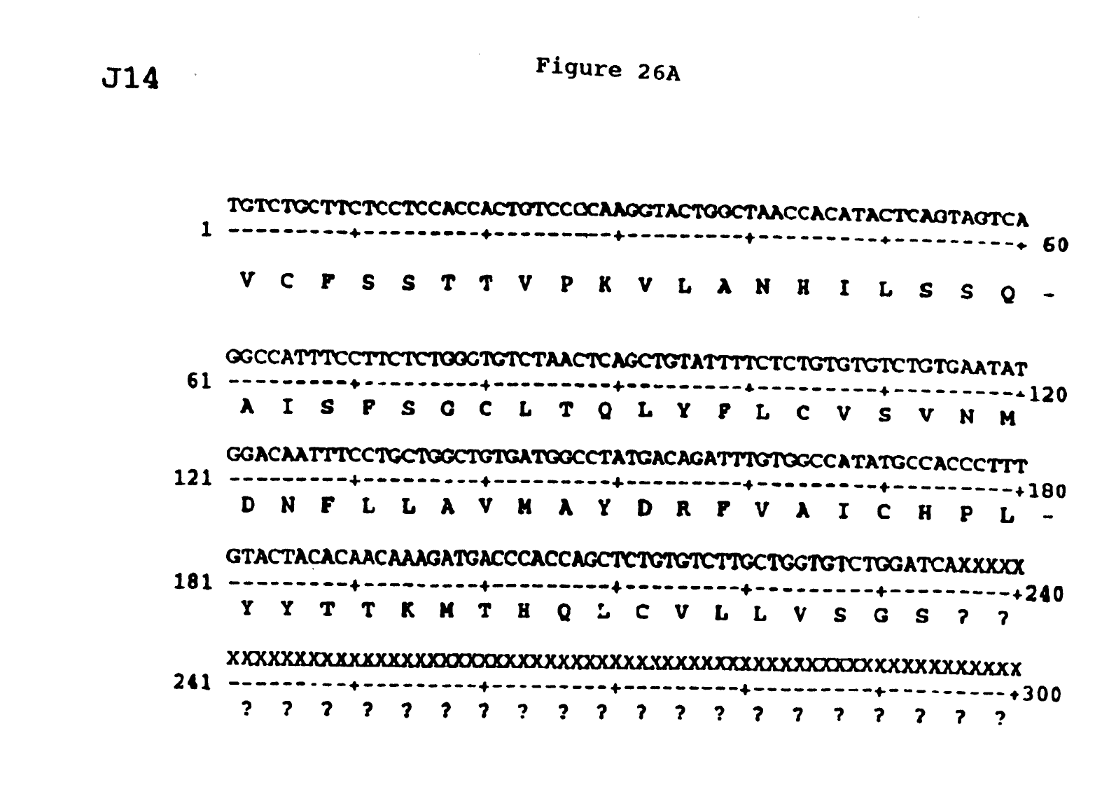

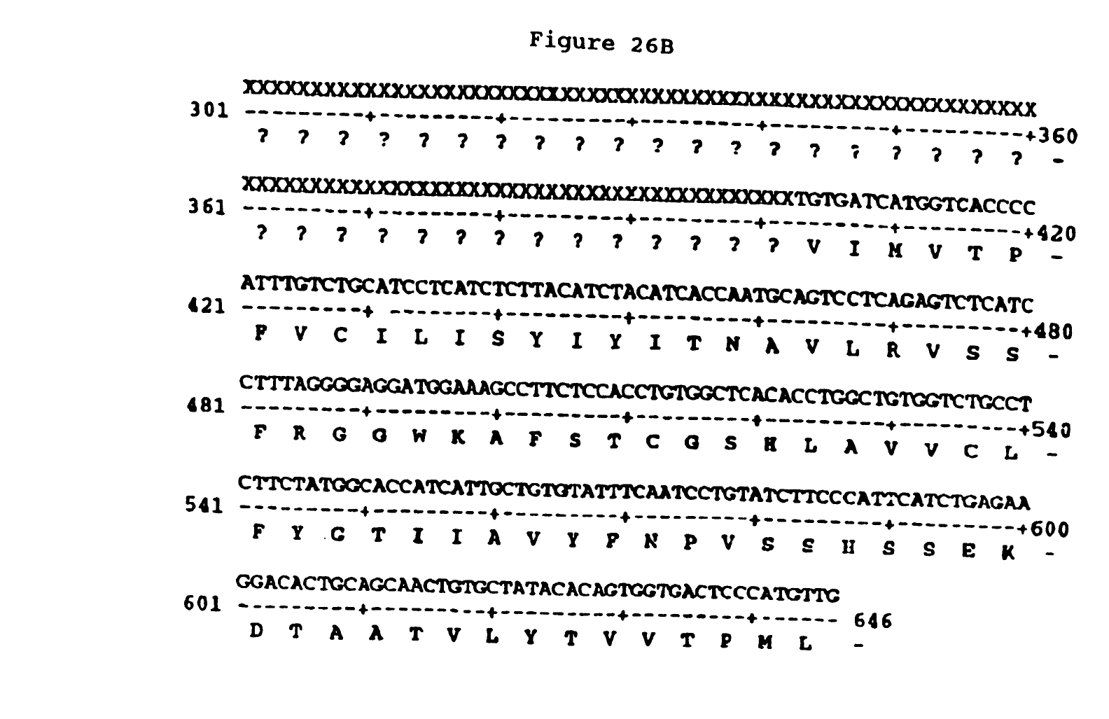

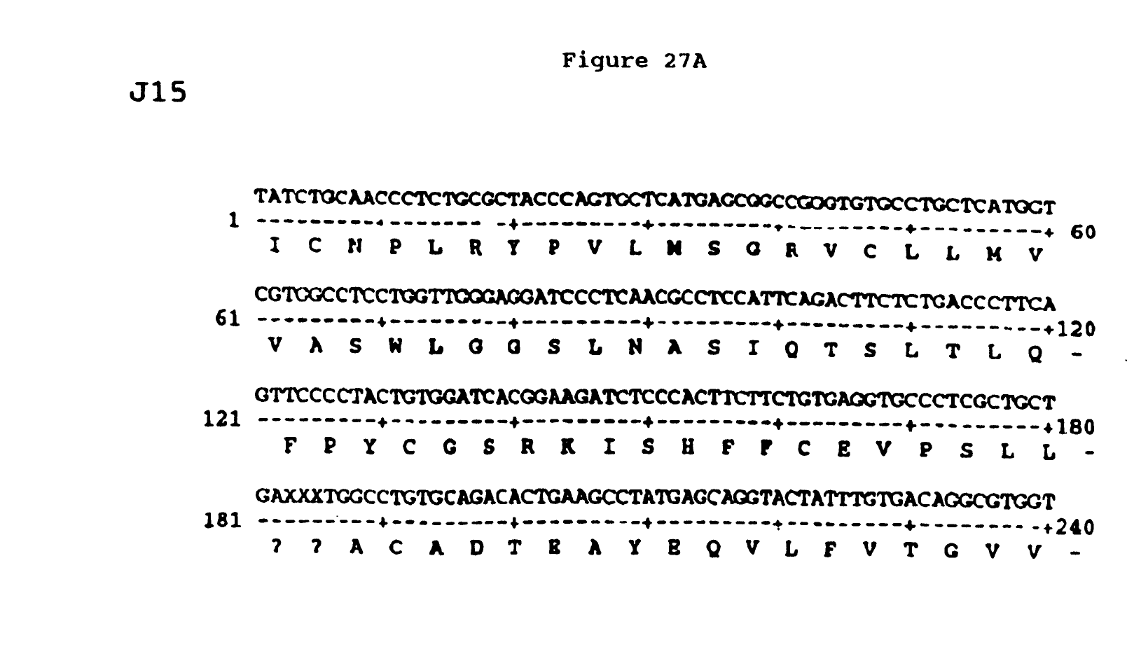

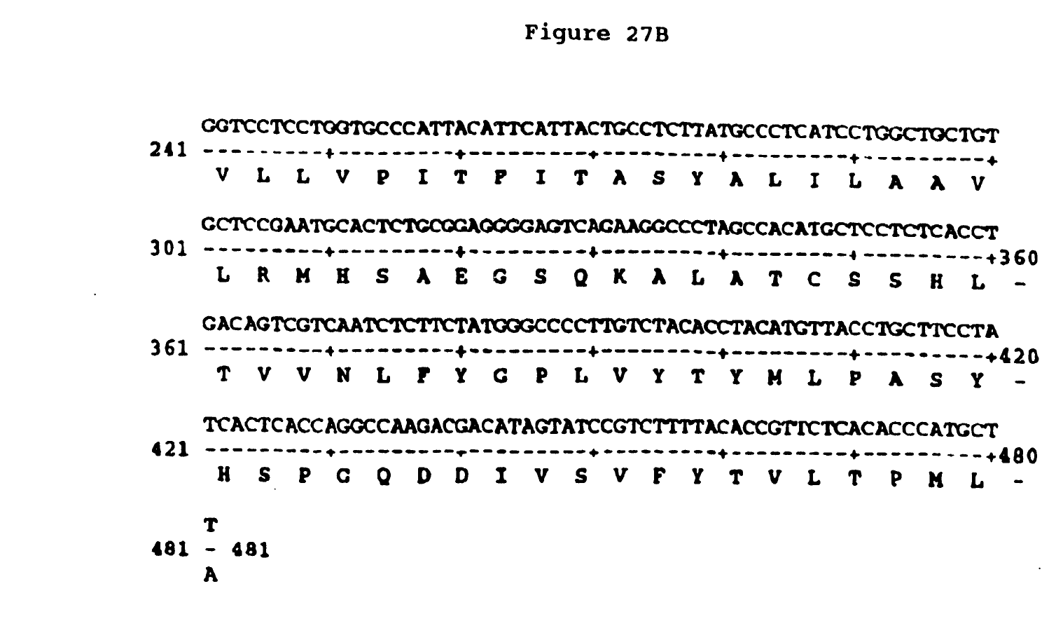

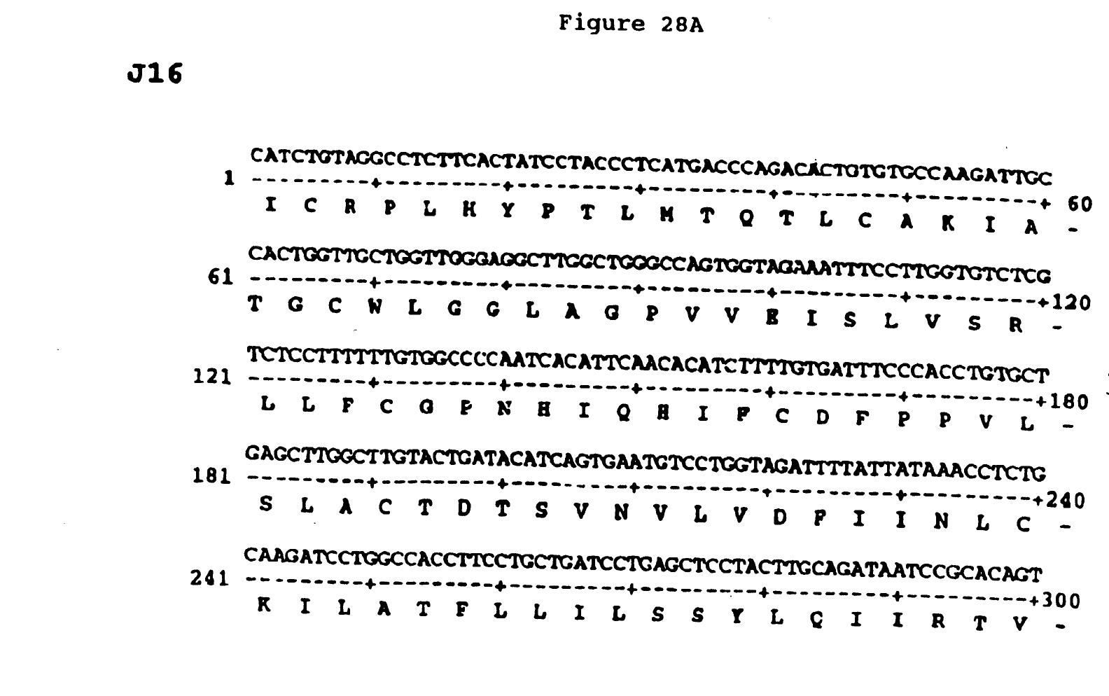

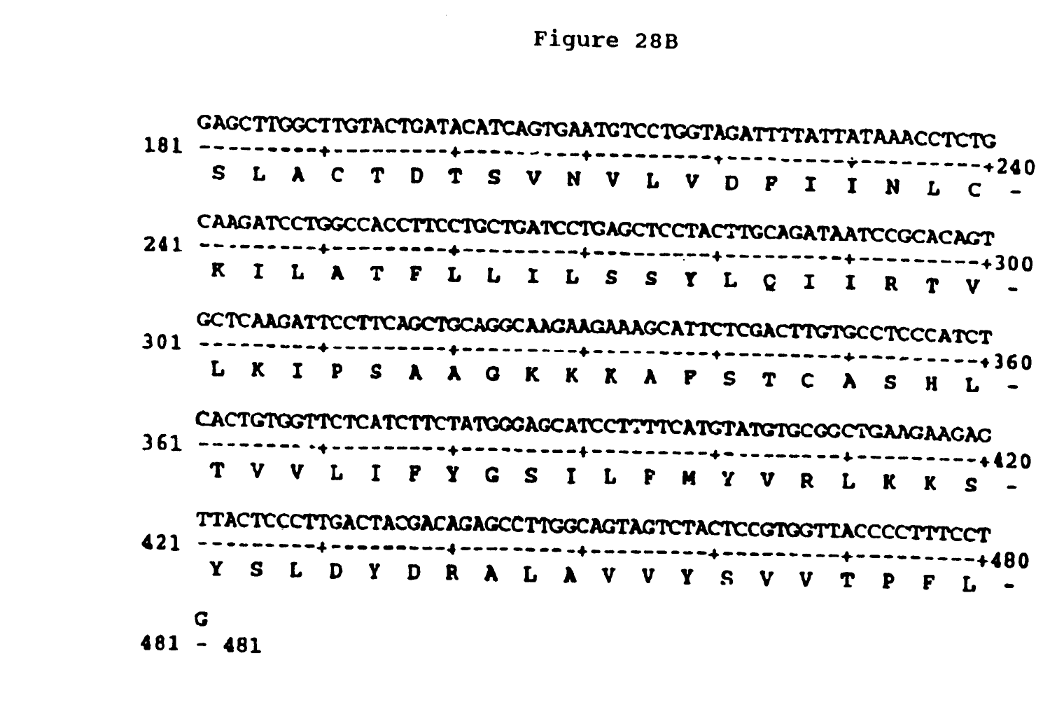

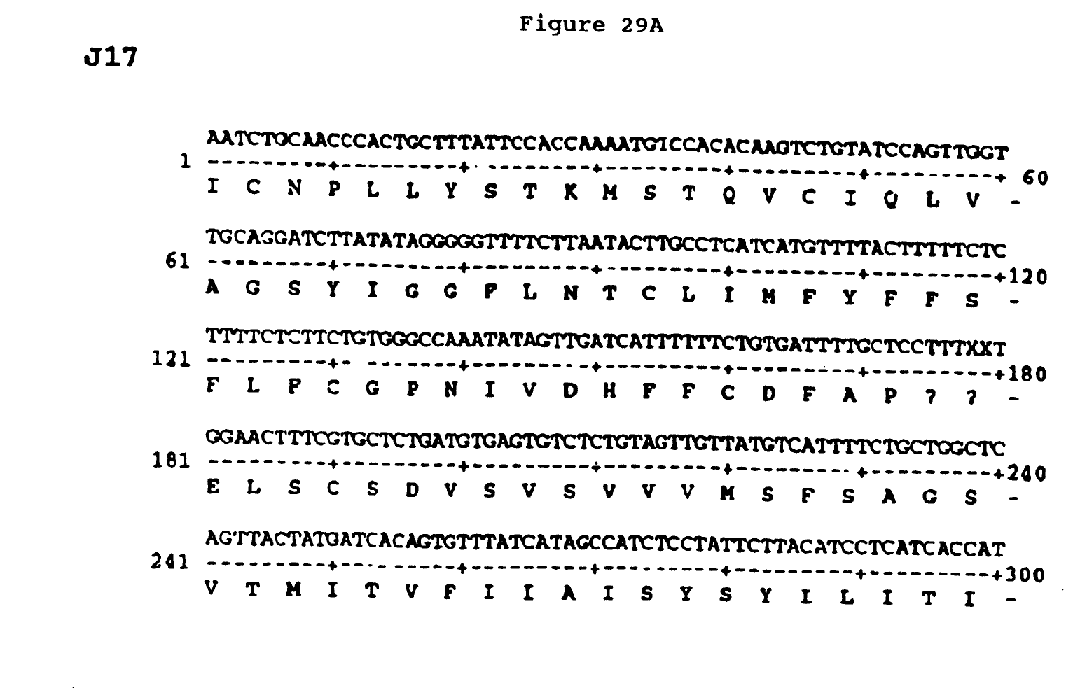

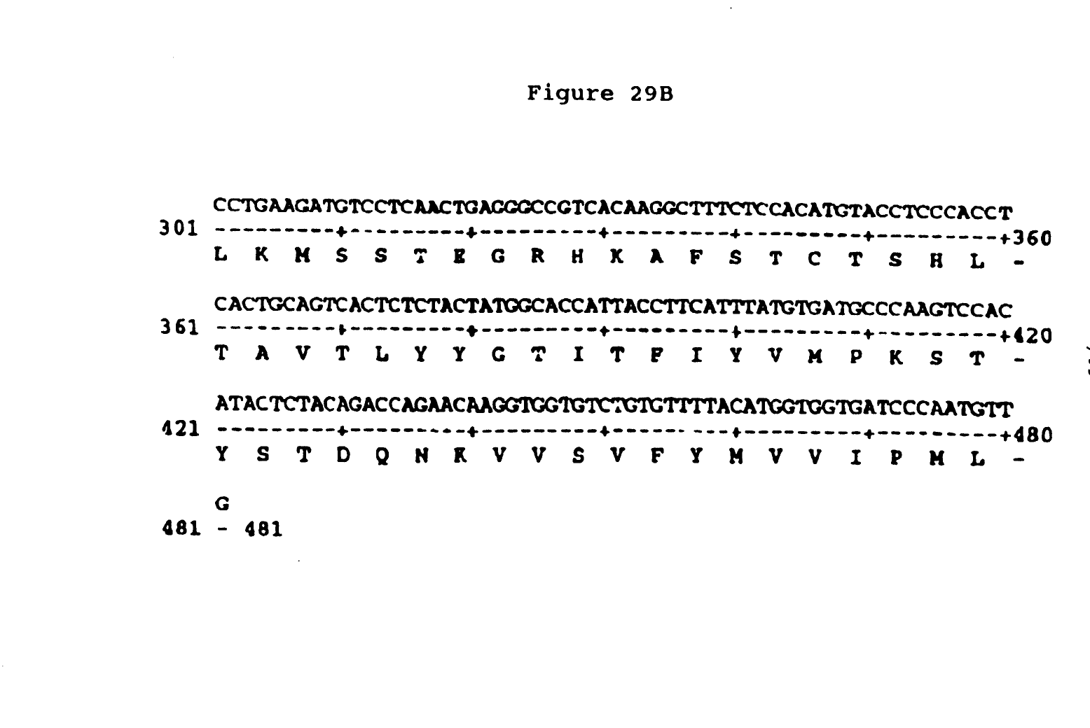

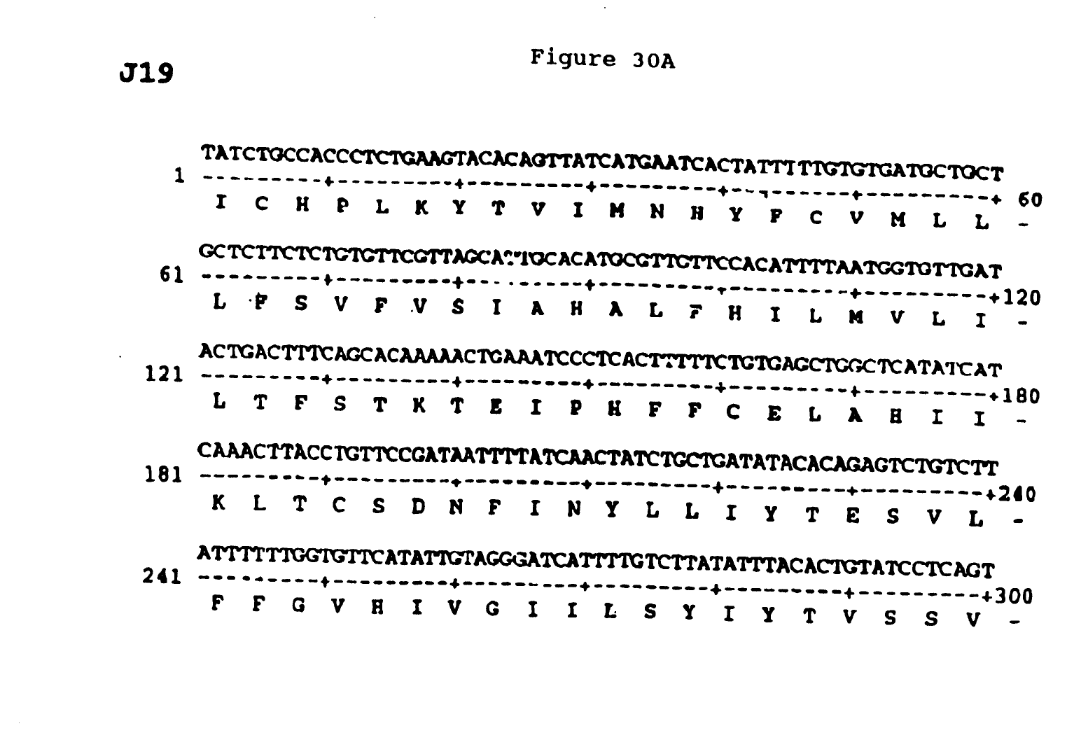

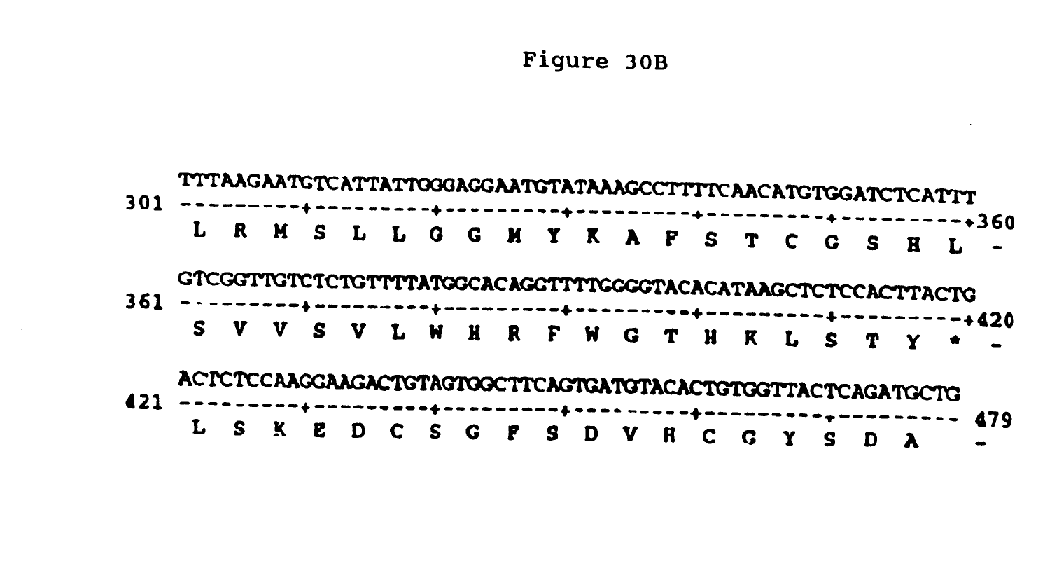

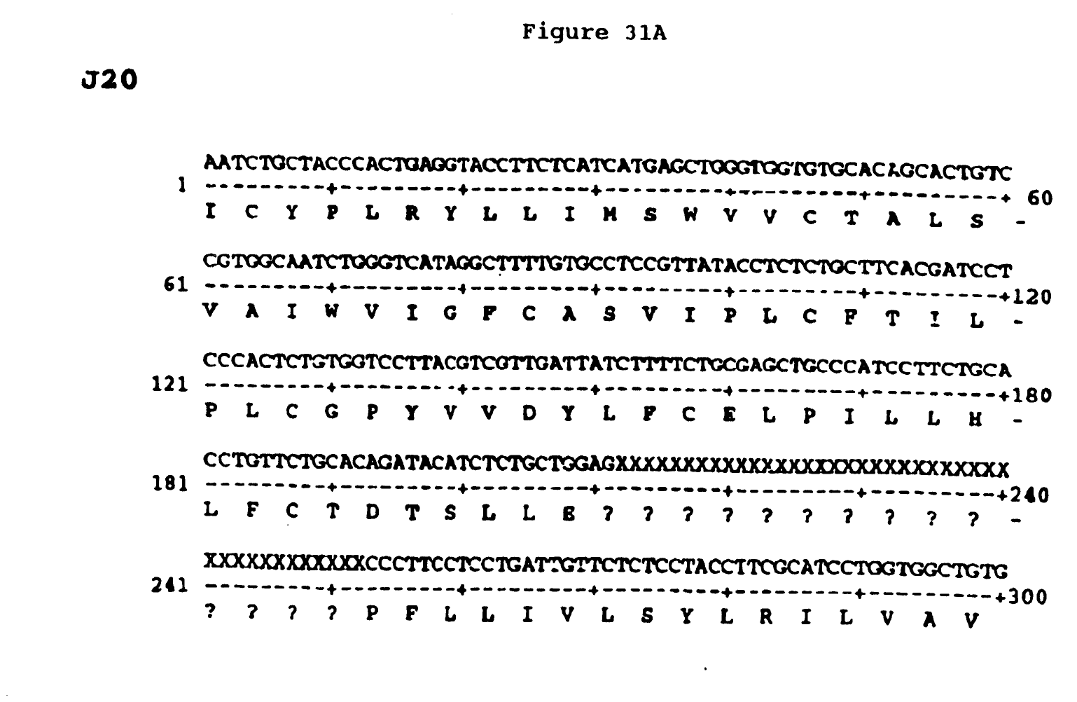

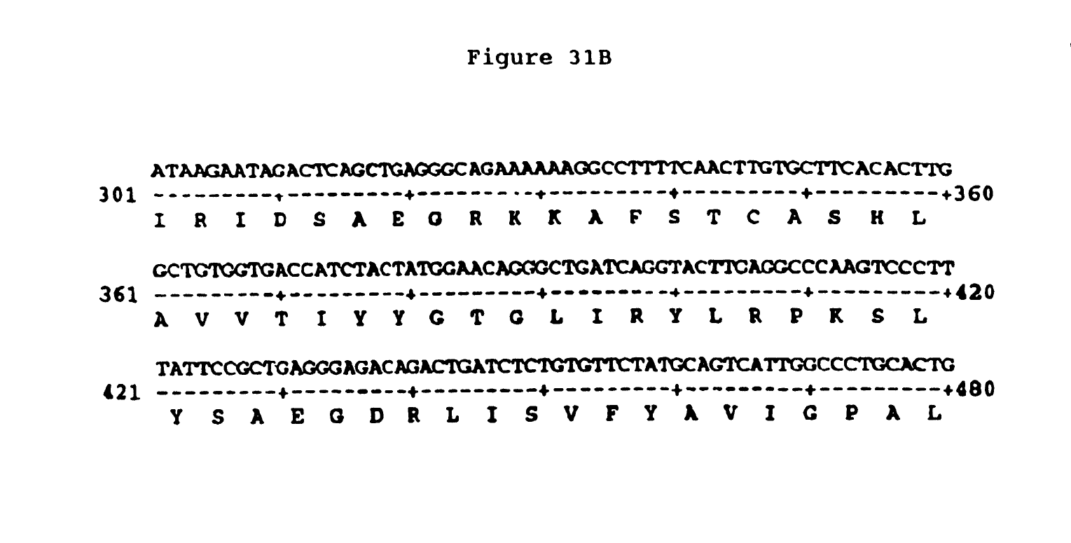

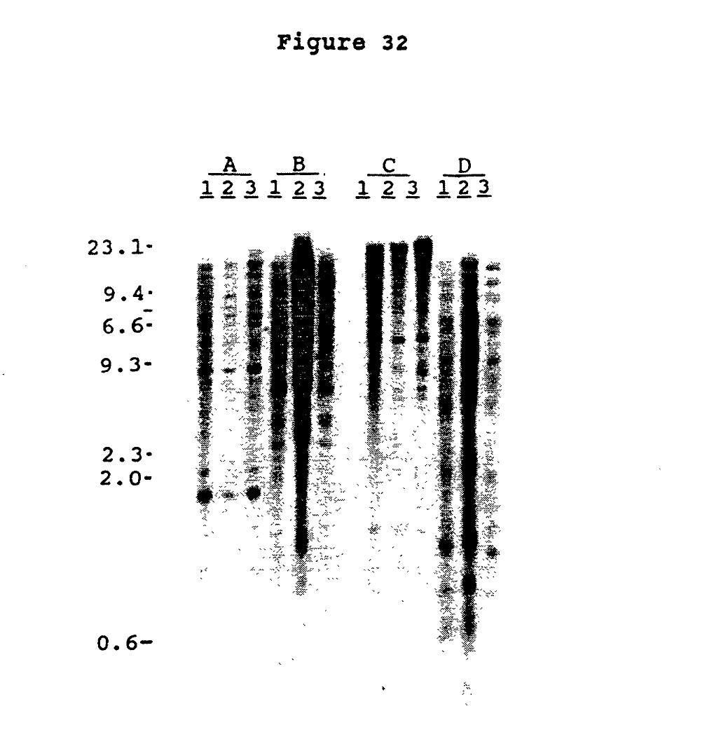

(A). The Olfactory Neuroepithelium. The initial event in odor perception occurs in the nasal cavity in a specialized neuroepithelium which is diagramed here. Odors are believed to interact with specific receptors on the cilia of olfactory sensory neurons. The signal generated by these initial binding events are propagated by olfactory neuron axons to the olfactory bulb.

(B). A Pathway of Olfactory Signal Transduction. In this scheme, the binding of an odorant molecule to an odor-specific transmembrane receptor leads to the interaction of the receptor with a GTP-binding protein (G_{S[olf]}) This interaction, in turn, leads to the release of the GTP-coupled a-subunit of the G-protein, which then stimulates adenylyl cyclase to produce elevated levels of cAMP. The increase in cAMP opens nucleotide-gated cation channels, thus causing an alteration in membrane potential.

FIGS. 2A-B. A PCR Amplification Product Containing Multiple Species of DNA. cDNA prepared from olfactory epithelium RNA was subjected to PCR amplification with a series of different primer oligonucleotides and the DNA products of appropriate size were isolated, further amplified by PCR, and size fractionated on agarose gels (A) (For details, see text). Each of these semipurified PCR products was digested with the restriction enzyme, Hinf I, and analyzed by agarose gel electrophoresis. Lanes marked “M” contain size markers of 23.1, 9.4, 5.6, 4.4, 2.3, 2.0, 1.35, 1.08, 0.87, 0.60, 0.31, 0.28, 0.23, 0.19, 0.12 and 0.07 kb. (B). Twenty-two of the 64 PCR products that were isolated and digested with Hinf I are shown here. Digestion of one of these, PCR 13, yielded a large number of fragments whose sizes summed to a value much greater than that of the undigested PCR 13 DNA, indicating that PCR 13 might contain multiple species of DNA which are representatives of a multigene family.

FIG. 3. Northern Blot Analysis with a Mixture of Twenty Probes. One μg of polyA+ RNA isolated from rat olfactory epithelium, brain, or spleen was size-fractionated in formaldehyde agarose, blotted onto a nylon membrane, and hybridized with a ³²P-labeled mixture of segments of 20 cDNA clones. The DNA segments were obtained by PCR using primers homologous to transmembrane domains 2 and 7.

FIGS. 4A-M. The Protein Sequences Encoded by Ten Divergent cDNA Clones. Ten divergent cDNA clones were subjected to DNA sequence analyses and the protein sequence encoded by each was determined (SEQ ID Nos: 71-80). Amino acid residues which are conserved in 60% or more of the proteins are shaded. The presence of seven hydrophobic domains (I-VII), as well as short conserved motifs shared with other members of the superfamily, demonstrate that these proteins belong to the seven transmembrane (TM) domain protein superfamily. The transmembrane regions are indicated by labeled lines (I-VII) above the sequences. Motifs conserved among members of the family of olfactory proteins include those indicated by underlining below the sequences. In addition, the DRY motif C-terminal to TM3 is common to many members of the G-protein-coupled superfamily. However, all of the proteins shown here share sequence motifs not found in other members of this superfamily and are clearly members of a novel family of proteins.

FIG. 5. Positions of Greatest Variability in the Olfactory Protein Family. In this diagram, the protein encoded by cDNA clone I15 is shown traversing the plasma membrane seven times with its N-terminus located extracellularly, and its C-terminus intracellularly. The vertical cylinders delineate the seven putative a-helices spanning the membrane. Positions at which 60% or more of the 10 clones shown in FIG. 4 share the same residue as I15 are shown as white balls. More variable residues are shown as black balls. The high degree of variability encountered in transmembrane domains III, IV, and V is evident in this schematic.

FIGS. 6A-D. The Presence of Subfamilies in a Divergent Multigene Family. Partial nucleotide sequences and deduced protein sequences were obtained for 18 different cDNA clones. Transmembrane domain V along with the flanking loop sequences, including the entire cytoplasmic loop between transmembrane domains V and VI, are shown here for each protein. Amino acid residues found in 60% or more of the clones in a given position are shaded (A). This region of the olfactory proteins (particularly transmembrane domain V) appears to be highly variable (see FIG. 4). These proteins, however, can be grouped into subfamilies (B,C,D) in which the individual subfamily members share considerable homology in this divergent region of the protein.

FIG. 7. Southern Blot Analyses with Non-crosshybridizing Fragments of Divergent cDNAs. Five μg of rat liver DNA was digested with Eco RI (A) or Hind III (B), electrophoresed in 0.75% agarose, blotted onto a nylon membrane, and hybridized to the ³²P-labeled probes indicated. The probes used were PCR-generated fragments of: 1, clone F9 (identical to F12 in FIG. 4); 2, F5; 3, F6; 4, I3; 5, I7; 6, I14; or 7, I15. The lane labeled “1-7” was hybridized to a mixture of the seven probes. The probes used showed either no crosshybridization or only trace crosshybridization with one another. The size markers on the left correspond to the four blots on the left (1-4) whereas the marker positions noted on the right correspond to the four blots on the right (5-7, “1-7”)

FIG. 8. Northern Blot Analysis with a Mix of Seven Divergent Clones. One μg of polyA+ RNA from each of the tissues shown was size-fractionated, blotted onto a nylon membrane, and hybridized with a ³²P-labeled mixture of segments of seven divergent cDNA clones (see Legend to FIG. 7).

FIGS. 9A-D. The nucleic acid and amino acid sequence of clone F3 (SEQ ID NO: 2 and SEQ ID NO: 71, respectively).

FIGS. 10A-D. The nucleic acid and amino acid sequence of clone F5 (SEQ ID NO: 3 and SEQ ID NO: 72, respectively).

FIGS. 11A-D. The nucleic acid and amino acid sequence of clone F6 (SEQ ID NO: 4 and SEQ ID NO: 73, respectively)

FIGS. 12A-D. The nucleic acid and amino acid sequence of clone F12 (SEQ ID NO: 1 and SEQ ID NO: 74, respectively).

FIGS. 13A-C. Partial nucleic acid and amino acid sequence of clone I3. Full nucleic acid and amino acid sequence of clone I3 are indicated in SEQ ID NO: 7 and SEQ ID NO: 75, respectively.

FIGS. 14A-D. The nucleic acid and amino acid sequence of clone I7 (SEQ ID NO: 8 and SEQ ID NO: 76, respectively).

FIGS. 15A-D. The nucleic acid and amino acid sequence of clone I8 (SEQ ID NO: 9 and SEQ ID NO: 77, respectively).

FIGS. 16A-D. The nucleic acid and amino acid sequence of clone I9 (SEQ ID NO: 10 and SEQ ID NO: 78, respectively).

FIGS. 17A-D. The nucleic acid and amino acid sequence of clone I14 (SEQ ID NO: 5 and SEQ ID NO: 79, respectively).

FIGS. 18A-D. The nucleic acid and amino acid sequence of clone I15 (SEQ ID NO: 6 and SEQ ID NO: 80, respectively).

FIGS. 19A-D. The nucleic acid and amino acid sequence of human clone H5 (SEQ ID NO: 11 and SEQ ID NO: 12, respectively).

FIGS. 20A-C. The nucleic acid and amino acid sequence of clone J1, where the reading frame starts at nucleotide position 2 (SEQ ID NO: 13 and SEQ ID NO: 14, respectively).

FIGS. 21A-B. The nucleic acid and amino acid sequence of clone J2 (SEQ ID NO: 15 and SEQ ID NO: 16, respectively).

FIGS. 22A-B. The nucleic acid and amino acid sequence of clone J4, where the reading frame starts at nucleotide position 2 (SEQ ID NO: 17 and SEQ ID NO: 18, respectively).

FIGS. 23A-B. The nucleic acid and amino acid sequence of clone J7, where the reading frame starts at nucleotide position 2 (SEQ ID NO: 19 and SEQ ID NO: 20, respectively).

FIGS. 24A-B. The nucleic acid and amino acid sequence of clone J8, where the reading frame starts at nucleotide position 2 (SEQ ID NO: 21 and SEQ ID NO: 22, respectively).

FIGS. 25A-C. The nucleic acid and amino acid sequence of clone J11 (SEQ ID NO: 23 and SEQ ID NO: 24, respectively).

FIGS. 26A-B. The nucleic acid and amino acid sequence of clone J14, where the reading frame starts at nucleotide position 2 (SEQ ID NO: 25 and SEQ ID NO: 26, respectively).

FIGS. 27A-B. The nucleic acid and amino acid sequence of clone J15, where the reading frame starts at nucleotide position 2 (SEQ ID NO: 27 and SEQ ID NO: 28, respectively).

FIGS. 28A-B. The nucleic acid and amino acid sequence of clone J16, where the reading frame starts at nucleotide position 2 (SEQ ID NO: 29 and SEQ ID NO: 30, respectively).

FIGS. 29A-B. The nucleic acid and amino acid sequence of clone J17, where the reading frame starts at nucleotide position 2 (SEQ ID NO: 31 and SEQ ID NO: 32, respectively).

FIGS. 30A-B. The nucleic acid and amino acid sequence of clone J19, where the reading frame starts at nucleotide position 2 (SEQ ID NO: 33 and SEQ ID NO: 34, respectively) The amino acid sequence after the stop codon is given in SEQ ID NO: 54.

FIGS. 31A-B. The nucleic acid and amino acid sequence of clone J20₇ where the reading frame starts at nucleotide position 2 (SEQ ID NO: 35 and SEQ ID NO: 36, respectively). FIG. 32. SOUTHERN BLOT: Five micrograms of DNA isolated from 1. Human placenta, 2. NCI-H-1011 neuroblastoma cells, or 3. CHP 134 neuroblastoma cells were treated with the restriction enzyme A. Eco RI, B. Hind III, C. Bam HI, or D. Pst I, and then electrophoresed on an agarose gel and blotted onto a nylon membrane. The blotted DNA was hybridized to the ³²P-labeled H3/H5 sequence. An autoradiograph of the hybridized blot is shown with the sizes of co-electrophoresed size markers noted in kilobases.

### DETAILED DESCRIPTION OF THE INVENTION

Throughout this application, the following standard abbreviations are used to indicate specific amino acids:

| 3-character | | 1-character |
|---|---|---|
| abbreviation | Amino Acid | abbreviation |
| Ala | Alanine | A |
| Arg | Arginine | R |
| Asn | Asparagine | N |
| Asp | Aspartic Acid | D |
| Cys | Cysteine | C |
| Gln | Glutamine | Q |
| Glu | Glutamic Acid | E |
| Gly | Glycine | G |
| His | Histidine | H |
| Ile | Isoleucine | I |
| Leu | Leucine | L |
| Lys | Lysine | K |
| Met | Methionine | M |
| Phe | Phenylalanine | F |
| Pro | Proline | P |
| Ser | Serine | S |
| Thr | Threonine | T |
| Trp | Tryptophane | W |
| Tyr | Tyrosine | Y |
| Val | Valine | V |
| Asx | Asparagine/ | B |
| | Aspartic Acid | |
| Glx | Glutamine/ | Z |
| | Glutamic Acid | |
| *** | (End) | * |
| Xxx | Any amino acid or as | X |
| | specified. | |

The following standard abbreviations are used to indicate specific nucleotide bases:
A = adenine;
C = cytosine;
G = guanine;
T = thymine.
I = inosine (modified nucleotide).

Having due regard to the preceding definitions, the invention provides an isolated nucleic acid molecule encoding an odorant receptor protein, wherein the receptor protein comprises seven transmembrane domains, and is further characterized by at least one of the following characteristics:

(a) the loop between the first transmembrane domain and the second transmembrane domain, and the second transmembrane domain together comprise consecutive amino acids having the following sequence:

-L, X, X, P, M, Y, X, F, L- (SEQ ID NO: 55);

(b) the third transmembrane domain, and the loop between the third transmembrane domain and the fourth transmembrane domain together comprise consecutive amino acids having one of the following sequences:

-M, X, Y, D, R, X, X, A, I, C- (SEQ ID NO: 57); or

-D, R, X, X, A, I, C- (SEQ ID NO: 59);

(c) the loop between the fifth transmembrane domain and the sixth transmembrane domain, and the sixth transmembrane domain together comprise consecutive amino acids having one of the following sequences:

-K or R, X, F, S, T, C, X, S, H- (SEQ ID NO: 61); or

-F, S, T, C, X, S, H- (SEQ ID NO: 63); or

(d) the seventh transmembrane domain and the C-terminal domain together comprise consecutive amino acids having one of the following sequences:

-P, X, X, N, P, X, I, Y, X, L, R, N- (SEQ ID NO: 65); or

-P, X, X, N, P, X, I, Y- (SEQ ID NO: 67); or

-N, P, X, I, Y, X, L, R, N- (SEQ ID NO: 69);

wherein X is any amino acid.

In one embodiment:

(a) the loop between the first transmembrane domain and the second transmembrane domain, and the second transmembrane domain together comprise consecutive amino acids having the following sequence:

-L, H or Q, K or M or T, PMY, F or L, FL- (SEQ ID NO: 56);

(b) the third transmembrane domain, and the loop between the third transmembrane domain and the fourth transmembrane domain together comprise consecutive amino acids having one of the following sequences:

-M, A or S, YDR, F or Y, L or V, AIC- (SEQ ID NO: 58); or

-DR, F or Y, L or V, AIC- (SEQ ID NO: 60);

(c) the loop between the fifth transmembrane domain and the sixth transmembrane domain, and the sixth transmembrane domain together comprise consecutive amino acids having one of the following sequences:

-K or R, A or I or S or V, FSTC, A or G or S, SH- (SEQ ID NO: 62); or

-FSTC, A or G or S, SH- (SEQ ID NO: 64); or

(d) the seventh transmembrane domain and the C-terminal domain together comprise consecutive amino acids having one of the following sequences:

-P, M or L or V, F or L or V, NP, F or I, IY, C or S or T, LRN-(SEQ ID NO: 66); or

-P, M or L or V, F or L or V, NP, F or I, IY- (SEQ ID NO: 68); or

-NP, F or I, IY, C or S or T, LRN- (SEQ ID NO: 70).

In one embodiment of the isolated nucleic acid molecule described herein, the receptor protein is characterized by at least two of the characteristics (a) through (d). In one embodiment, the receptor protein is characterized by at least three of the characteristics (a) through (d). In one embodiment, the receptor protein is characterized by all of the characteristics (a) through (d).

The invention provides an isolated nucleic acid molecule encoding an odorant receptor protein, wherein the nucleic acid molecule encodes a protein selected from the group consisting of:

(a) an odorant receptor protein comprising consecutive amino acids having a sequence identical to that beginning with methionine at position 1 and ending with tyrosine at position 333 as set forth in row F3 of FIGS. 4A to 4M (SEQ ID NO: 71),

(b) an odorant receptor protein comprising consecutive amino acids having a sequence identical to that beginning with methionine at position 1 and ending with glutamine at position 313 as set forth in row F5 of FIGS. 4A to 4L (SEQ ID NO: 72),

(c) an odorant receptor protein comprising consecutive amino acids having a sequence identical to that beginning with methionine at position 1 and ending with lysine at position 311 as set forth in row F6 of FIGS. 4A to 4L (SEQ ID NO: 73),

(d) an odorant receptor protein comprising consecutive amino acids having a sequence identical to that beginning with methionine at position 1 and ending with glycine at position 317 as set forth in row F12 of FIGS. 4A to 4L (SEQ ID NO: 74),

(e) an odorant receptor protein comprising consecutive amino acids having a sequence identical to that beginning with methionine at position 1 and ending with leucine at position 310 as set forth in row I3 of FIGS. 4A to 4L (SEQ ID NO: 75),

(f) an odorant receptor protein comprising consecutive amino acids having a sequence identical to that beginning with methionine at position 1 and ending with glycine at position 327 as set forth in row I7 of FIGS. 4A to 4L (SEQ ID NO: 76),

(g) an odorant receptor protein comprising consecutive amino acids having a sequence identical to that beginning with methionine at position 1 and ending with tryptophan at position 312 as set forth in row I8 of FIGS. 4A to 4L (SEQ ID NO: 77),

(h) an odorant receptor protein comprising consecutive amino acids having a sequence identical to that beginning with methionine at position 1 and ending with leucine at position 314 as set forth in row I9 of FIGS. 4A to 4L (SEQ ID NO: 78),

(i) an odorant receptor protein comprising consecutive amino acids having a sequence identical to that beginning with methionine at position 1 and ending with leucine at position 312 as set forth in row I14 of FIGS. 4A to 4L (SEQ ID NO: 79),

(j) an odorant receptor protein comprising consecutive amino acids having a sequence identical to that beginning with methionine at position 1 and ending with leucine at position 314 as set forth in row I15 of FIGS. 4A to 4L (SEQ ID NO: 80), and

(k) an odorant receptor protein that shares from 40-80% amino acid identity with any one of the proteins of (a)-(j), comprises seven transmembrane domains, and is further characterized by at least one of the following characteristics:

(i) the loop between the first transmembrane domain and the second transmembrane domain, and the second transmembrane domain together comprise consecutive amino acids having the following sequence: -L, X, X, P, M, Y, X, F, L- (SEQ ID NO: 55);

(ii) the third transmembrane domain, and the loop between the third transmembrane domain and the fourth transmembrane domain together comprise consecutive amino acids having one of the following sequences:

-M, X, Y, D, R, X, X, A, I, C- (SEQ ID NO: 57); or

-D, R, X, X, A, I, C- (SEQ ID NO: 59);

(iii) the loop between the fifth transmembrane domain and the sixth transmembrane domain, and the sixth transmembrane domain together comprise consecutive amino acids having one of the following sequences:

-K or R, X, F, S, T, C, X, S, H- (SEQ ID NO: 61); or

-F, S, T, C, X, S, H- (SEQ ID NO: 63); or

(iv) the seventh transmembrane domain and the C-terminal domain together comprise consecutive amino acids having one of the following sequences:

-P, X, X, N, P, X, I, Y, X, L, R, N- (SEQ ID NO: 65); or

-P, X, X, N, P, X, I, Y- (SEQ ID NO: 67); or

-N, P, X, I, Y, X, L, R, N- (SEQ ID NO: 69);

wherein X is any amino acid.

In one embodiment:

(i) the loop between the first transmembrane domain and the second transmembrane domain, and the second transmembrane domain together comprise consecutive amino acids having the following sequence:

-L, H or Q, K or M or T, PMY, F or L, FL- (SEQ ID NO: 56);

(ii) the third transmembrane domain, and the loop between the third transmembrane domain and the fourth transmembrane domain together comprise consecutive amino acids having one of the following sequences:

-M, A or S, YDR, F or Y, L or V, AIC- (SEQ ID NO: 58); or

-DR, F or Y, L or V, AIC- (SEQ ID NO: 60);

(iii) the loop between the fifth transmembrane domain and the sixth transmembrane domain, and the sixth transmembrane domain together comprise consecutive amino acids having one of the following sequences:

-K or R, A or I or S or V, FSTC, A or G or S, SH- (SEQ ID NO: 62); or

-FSTC, A or G or S, SH- (SEQ ID NO: 64); or

(iv) the seventh transmembrane domain and the C-terminal domain together comprise consecutive amino acids having one of the following sequences:

-P, M or L or V, F or L or V, NP, F or I, IY, C or S or T, LRN- (SEQ ID NO: 66); or

-P, M or L or V, F or L or V, NP, F or I, IY- (SEQ ID NO: 68); or

-NP, F or I, IY, C or S or T, LRN- (SEQ ID NO: 70).

The invention provides an isolated nucleic acid molecule encoding an odorant receptor protein, wherein the nucleic acid molecule comprises a nucleic acid sequence which can be amplified by polymerase chain reaction using:

(a) any one of 5′ primers Al (SEQ ID NO: 37), A2 (SEQ ID NO: 38), A3 (SEQ ID NO: 39), A4 (SEQ ID NO: 40), or A5 (SEQ ID NO: 41); and

(b) any one of 3′ primers Bl (SEQ ID NO: 42), B2 (SEQ ID NO: 43), B3 (SEQ ID NO: 44), B4 (SEQ ID NO: 45), B5 (SEQ ID NO: 46), or B6 (SEQ ID NO: 47).

The invention provides an isolated nucleic acid molecule encoding an odorant receptor protein, wherein the nucleic acid molecule comprises:

(a) a nucleic acid sequence given in any one of FIGS. 9 to 31 (SEQ ID Nos: 1-10, 11, 13, 15, 17, 19, 21, 23, 25, 27, 29, 31, 33, or 35); or

(b) a nucleic acid sequence degenerate to a sequence of (a) as a result of the genetic code.

In one embodiment, the odorant receptor protein encoded by any of the isolated nucleic acid molecules described herein comprises seven transmembrane domains. In one embodiment, the loop between the fifth and sixth transmembrane domains consists of 17 amino acids.

An odorant receptor is a receptor which binds an odorant ligand and includes but is not limited to pheromone receptors. An odorant ligand may include, but is not limited to, molecules which interact with the olfactory sensory neuron, molecules which interact with the olfactory cilia, pheromones, and molecules which interact with structures within the vomeronasal organ.

In one embodiment, the odorant receptor protein encoded by any of the isolated nucleic acid molecules described herein is a vertebrate odorant receptor. In one embodiment, the vertebrate odorant receptor is a fish odorant receptor or a mammalian odorant receptor. In one embodiment, the mammalian odorant receptor is a human odorant receptor, a rat odorant receptor, a mouse odorant receptor or a dog odorant receptor.

In one embodiment, the isolated nucleic acid molecule described herein is deoxyribonucleic acid (DNA). In one embodiment, the DNA is cDNA.

In an embodiment, a human odorant receptor CDNA sequence and the corresponding protein are isolated (SEQ ID Nos: 11 and 12) (FIG. 19)

In another embodiment, pheromone receptors are isolated and shown as clones J1 (SEQ ID NOs: 13 and 14), J2 (SEQ ID NOs: 15 and 16), J4 (SEQ ID NOs: 17 and 18), J7 (SEQ ID NOs: 19 and 20), J8 (SEQ ID NOs: 21 and 22), J11 (SEQ ID NOs: 23 and 24), J14 (SEQ ID NOs: 25 and 26), J15 (SEQ ID NOs: 27 and 28), J16 (SEQ ID NOs: 29 and 30), J17 (SEQ ID NOs: 31 and 32), J19 (SEQ ID NOs: 33 and 34) and J20 (SEQ ID NOs: 35 and 36)(FIGS. 20-31).

The invention provides a vector comprising any of the isolated nucleic acid molecules described herein. In one embodiment, the vector additionally comprises elements necessary for replication and expression in a suitable host. Such expression vectors are well known in the art. Suitable hosts are well known in the art and include without limitation bacterial hosts such as E. coli, animal hosts such as CHO cells, insect cells, yeast cells and the like.

The invention provides a purified odorant receptor protein encoded by any of the isolated nucleic acid molecules described herein. Such proteins may be prepared by expression of the aforementioned expression vectors in suitable host cells, and recovery and purification of the receptors using methods well known in the art. Examples of such proteins include those having the amino acid sequences shown in FIGS. 9-31.

The invention provides methods of transforming cells which comprises transfecting a suitable host cell with a suitable expression vector containing nucleic acid encoding the odorant receptor. Techniques for carrying out such transformations on cells are well known to those skilled in the art (41,42).

The invention provides a cell transfected with any of the vectors described herein. In one embodiment, the cell is an olfactory cell. In one embodiment, the cell is a non-olfactory cell. In one embodiment, prior to being transfected with the vector, the non-olfactory cell does not express an odorant receptor protein. One advantage of using such transformed non-olfactory cells is that the desired odorant receptor will be the only odorant receptor expressed on the cell's surface.

In order to obtain cell lines that express a single receptor type, standard procedures may be used to clone individual cDNAs or genes into expression vectors and then transfect the cloned sequences into mammalian cell lines. This approach has been used with sequences encoding some other members of the seven transmembrane domain superfamily including the 5-HT₁c serotonin receptor (43). The cited work illustrates how members of this superfamily transferred into cell lines may generate immortal cell lines that express high levels of the transfected receptor on the cell surface where it will bind ligand and that such abnormally expressed receptor molecules can transduce signals upon binding to ligand.

The invention provides a method of identifying a desired odorant ligand, which comprises contacting any of the non-olfactory cells described herein, which express on its cell surface a known odorant receptor, with a series of odorant ligands and determining which ligands bind to the known odorant receptor on the non-olfactory cell.

The invention provides a method of identifying a desired odorant receptor, which comprises contacting a series of any of the non-olfactory cells described herein with a known odorant ligand and determining which odorant receptor binds with the odorant ligand.

The invention provides a method of detecting an odor which comprises:

(a) identifying an odorant receptor which binds the desired odorant ligand identified by any of the methods described herein; and

(b) imbedding the receptor in a membrane such that when the odorant ligand binds with the receptor identified in (a) above, a detectable signal is produced.

In one embodiment of the described method, the desired odorant ligand is a pheromone. In different embodiments, the desired odorant ligand is the vapor emanating from cocaine, marijuana, heroin, hashish, angel dust, gasoline, natural gas, alcohol, decayed human flesh, gun powder, an explosive, a plastic explosive, or a firearm.

In different embodiments, the desired odorant ligand is a toxic fume, a noxious fume or a dangerous fume. In different embodiments, the membrane is a cell membrane, an olfactory cell membrane, or a synthetic membrane. In different embodiment of the methods described herein, the detectable signal is a color change, a phosphorescence, a radioactivity, a visual signal, or an auditory signal.

The invention provides a method of quantifying the amount of an odorant ligand present in a sample which comprises any of the methods described herein wherein the detectable signal is quantified.

The invention provides a method of developing fragrances, which comprises identifying a desired odorant receptor by any of the methods described herein, then contacting a non-olfactory cell, which has been transfected with an expression vector comprising an isolated nucleic acid molecule encoding the desired odorant receptor such that the receptor is expressed upon the surface of the non-olfactory cell, with a series of compounds to determine which compounds bind with the receptor.

The invention provides a method of identifying an odorant fingerprint, which comprises contacting a series of cells, which have been transformed such that each express a known odorant receptor encoded by any of the nucleic acid molecules described herein, with a desired sample containing one or more odorant ligand and determining the type and quantity of the odorant ligands present in the sample.

The invention provides a method of identifying a compound which inhibits an odorant receptor, which comprises contacting an odorant receptor encoded by any of the nucleic acid molecules described herein with a series of compounds and determining which compound inhibits interaction between the odorant receptor and an odorant ligand known to bind to the odorant receptor.

The invention provides a method for identifying an appetite suppressant compound, which comprises identifying a compound by any of the methods described herein wherein the odorant receptor is associated with the perception of food.

The invention provides a nasal spray for controlling appetite, which comprises a compound identified by the methods described herein in a suitable carrier.

The invention provides a method for controlling appetite in a subject, which comprises administering to the subject an amount of a compound identified by the methods described herein effective to control the subject's appetite. In one embodiment, the method comprises administering the compound to the subject's olfactory epithelium.

The invention provides a method of trapping odors, which comprises contacting a membrane comprising a plurality of a desired odorant receptor encoded by any of the nucleic acid molecules described herein with a sample comprising a desired odorant ligand such that the desired odorant ligand is absorbed by the binding of the odorant ligand to the odorant receptor.

The invention provides an odor trap, which comprises a membrane comprising a plurality of a desired odorant receptor encoded by any of the nucleic acid molecules described herein, such that a desired odorant ligand is absorbed by the binding of the odorant ligand to the odorant receptor.

The invention provides a method for controlling a pest population in an area, which comprises spraying the area with an odorant receptor ligand identified by the method described herein. In one embodiment, the odorant ligand is an alarm odorant ligand. In one embodiment, the odorant ligand interferes with an interaction between an odorant ligand and an odorant receptor associated with fertility. In different embodiments, the pest population is a population of rodents, mice, or rats.

The invention provides a method of promoting fertility in a subject which comprises administering to the subject an amount of an odorant ligand identified by the methods described herein effective to promote the subject's fertility. In one embodiment, the odorant ligand interacts with an odorant receptor associated with fertility. In one embodiment, the method comprises administering the odorant ligand to the subject's olfactory epithelium.

The invention provides a method of inhibiting fertility in a subject which comprises administering to the subject an amount of an odorant ligand identified by the methods described herein effective to inhibit the subject's fertility. In one embodiment, the odorant ligand inhibits an interaction between an odorant ligand and an odorant receptor associated with fertility. In one embodiment, the method comprises administering the odorant ligand to the subject's olfactory epithelium.

The invention provides the use of an odorant ligand identified by the methods described herein for the preparation of a pharmaceutical composition for controlling a pest population in a desired area by spraying the desired area with the identified odorant ligand. In one embodiment, the odorant ligand is an alarm odorant ligand. In different embodiments, the pest population is a population of rodents, mice, or rats.

The invention provides the use of an odorant ligand identified by the methods described herein for the preparation of a pharmaceutical composition for controlling a pest population. In one embodiment, the odorant ligand interferes with the interaction between odorant ligands and odorant receptors which are associated with fertility. In different embodiments, the pest population is a population of rodents, mice, or rats.

The invention provides the use of an odorant ligand identified by the methods described herein for the preparation of a pharmaceutical composition for promoting fertility. In one embodiment, the odorant ligand interacts with odorant receptors associated with fertility.

The invention provides the use of an odorant ligand identified by the methods described herein for the preparation of a pharmaceutical composition for inhibiting fertility. In one embodiment, the odorant ligand inhibits the interaction between odorant ligands and odorant receptors associated with fertility.

The invention provides the use of the compound identified by the methods described herein for the preparation of a pharmaceutical composition for controlling appetite in a subject.

The invention provides a pharmaceutical composition comprising any of the compounds or odorant ligands identified by any of the methods described herein and a pharmaceutically acceptable carrier. The phrase “pharmaceutically acceptable carrier” means any of the standard pharmaceutically acceptable carriers. Examples include, but are not limited to, phosphate buffered saline, physiological saline, water, and emulsions, such as oil/water emulsions. In one embodiment, the composition can be applied to a subject's olfactory epithelium.

This invention will be better understood from the Experimental Details which follow. However, one skilled in the art will readily appreciate that the specific methods and results discussed are merely illustrative of the invention as described more fully in the claims which follow thereafter.

Experimental Details

Materials And Methods

Polymerase Chain Reaction

RNA was prepared from the olfactory epithelia of Sprague Dawley rats according to Chirgwin et al. (40) or using RNAzol B (Cinna/Biotecx) and then treated with DNase I (0.1 unit/μg RNA) (Promega). In order to obtain cDNA, this RNA was incubated at 0.1 μg/yl with 5 μM random hexamers (Pharmacia), 1 mM each of DATP, dCTP, dGTP, TTP, and 2 units/μ1 RNase inhibitor (Promega) in 10 mM TrisCl (pH 8.3), 50 mM KCl, 2.5 mM MgCl₂, and 0.001% gelatin for 10 min. at 22° C., and then for a further 45 min. at 37° C. following the addition of 20 units/μl of Moloney murine leukemia virus reverse transcriptase (BRL). After heating at 95° C. for 3 min., cDNA prepared from 0.2 μg of RNA was used in each of a series of polymerase chain reactions (PCR) containing 10 mM TrisCl (pH 8.3), 50 mM KCl, 1.5 mM MgCl₂, 0.001% gelatin, 200 μM each of DATP, dCTP, dGTP, and TTP, 2.5 units Taq polymerase (Perkin Elmer Cetus), and 2 μM of each PCR primer. PCR reactions were performed according to the following schedule: 96° C. for 45 sec., 55° C. for 4 min. (or 45° C. for 2 min.), 72° C. for 3 min. with 6 sec. extension per cycle for 48 cycles. The primers used for PCR were a series of degenerate oligonucleotides made according to the amino acid sequences found in transmembrane domain 2 and 7 of a variety of different members of the 7 transmembrane domain protein superfamily (19). The regions used correspond to amino acids number 60-70 and 286-295 of clone I15 (FIG. 4). Each of five different 5′ primers were used in PCR reactions with each of six different 3′ primers. The 5′ primers had the sequences:

A1,
AA(T/C)T(G/A)(G/C)ATI(C/A)TI(G/C)TIAA(T/C)(C/T)TIG
CIGTIGCIGA (SEQ ID NO: 37);
A2,
AA(T/C)TA(T/C)TT(T/C)(C/A)TI(G/A)TIAA(T/C)CTIGCI
(T/C)TIGCIGA (SEQ ID NO: 38);
A3,
AA(T/C)(T/C)(T/A)ITT(T/C)(A/C)TIATI(T/A)CICTIGCIT
(G/C)IGCIGA (SEQ ID NO: 39);
A4,
(C/A)GITTI(C/T)TIATGTG(T/C)AA(C/T)CTI(T/A)(G/C)
(C/T)TT(T/C)GCIGA (SEQ ID NO: 40); and
A5,
ACIGTITA(T/C)ATIACICA(T/C)(C/T)TI(A/T)(C/G)IATIGCI
GA (SEQ ID NO: 41)
The 3′ primers were:
B1, CTGI(C/T)(G/T)(G/A)TTCATIA(A/T)I(A/C)(C/A)
(A/G)TAIA(T/C)IA(T/C)IGG(G/A)TT (SEQ ID NO: 42);
B2,
(G/T)(A/G)T(C/G)(G/A)TTIAG(A/G)CA(A/G)CA(A/G)TAIAT
IATIGG(G/A)TT (SEQ ID NO: 43);
B3,
TCIAT(G/A)TT(A/G)AAIGTIGT(A/G)TAIATIATIGG(G/A)TT
(SEQ ID NO: 44)
B4,
GC(C/T)TTIGT(A/G)AAIATIGC(A/G)TAIAG(G/A)AAIGG
(G/A)TT (SEQ ID NO: 45)
B5,
AA(A/G)TCIGG(G/A)(C/G)(T/A)ICGI(C/G)A(A/G)TAIAT
(C/G)AIIGG(G/A)TT (SEQ ID NO: 46); and
BG,(G/C)(A/T)I(G/C)(A/T)ICCIAC(A/G)AA(A/G)AA(A/G)
TAIAT(A/G)AAIGG(G/A)TT (SEQ ID NO: 47).

In the preceding formulae, each parenthesis encloses amino acids which are alternatives to one other, and each slash within such parentheses separates such alternative amino acids.

An aliquot of each PCR reaction was analyzed by agarose gel electrophoresis and bands of interest were amplified further by performing PCR reactions on pipette tip (approx. 1 μl) plugs of the agarose gels containing those DNAs. Aliquots of these semi-purified PCR products were digested with the restriction enzymes Hae III or Hinf I and the digestion products were compared with the undigested DNAs on agarose gels.

Isolation and Analysis of cDNA Clones

CDNA libraries were prepared according to standard procedures (41, 42) in the cloning vector, λZAP II (Stratagene) using poly A⁺ RNA prepared from Sprague Dawley rat epithelia (see above) or from an enriched population of olfactory neurons which had been obtained by a ‘panning’ procedure, using an antibody against the H blood group antigen (Chembiomed) found on a large percentage of rat olfactory neurons. In initial library screens, 8.5×10⁵ independent clones from the olfactory neuron library and 1.8×10⁶ clones from the olfactory epithelium library were screened (41) with a ³²P-labeled probe (prime-it, Stratagene) consisting of a pool of gel-isolated PCR products obtained using primers A4 and B6 (see above) in PCR reactions using as template, olfactory epithelium cDNA, rat liver DNA, or DNA prepared from the two cDNA libraries. In later library screens, a mixture of PCR products obtained from 20 cDNA clones with the A4 and B6 primers was used as probe (‘P1’ probe). In initial screens, phage clones were analyzed by PCR using primers A4 and B6 and those which showed the appropriate size species were purified. In later screens, all position clones were purified, but only those that could be amplified with the B6 primer and a primer specific for vector sequence were analyzed further. To obtain plasmids from the isolated phage clones, phagemid rescue was performed according to the instructions of the manufacturer of λZAP II (Stratagene). DNA sequence analysis was performed on plasmid DNAs using the Sequenase system (USB), initially with the A4 and B6 primers and later with oligonucleotide primers made according to sequences already obtained.

Northern and Southern Blot Analyses

For Northern blots, poly A⁺ RNAs from various tissues were prepared as described above or purchased from Clontech. One μg of each RNA was size fractionated on formaldehyde agarose gels and blotted onto nylon membranes (41, 42). For Southern blots, genomic DNA prepared from Sprague Dawley rat liver was digested with the restriction enzymes Eco RI or Hind III, size fractionated on agarose gels and blotted onto nylon membranes (41, 42). The membranes were dried at 80° C., and then prehybridized in 0.5 M sodium phosphate buffer (pH 7.3) containing 1% bovine serum albumin and 4% sodium dodecyl sulfate. Hybridization was carried out in the same buffer at 65°-70° C. for 14-20 hrs. with DNAs labeled with ³²P. For the first Northern blot shown, the ‘P1’ probe (see above under cDNA clone isolation) was used. For the second Northern blot shown, a mix of PCR fragments from seven divergent cDNA clones was used. For Southern blots, the region indicated in clone I15 by amino acids 118 through 251 was amplified from a series of divergent cDNA clones using PCR. The primers used for these reactions had the sequences:

P1, ATGGCITA(T/C)GA(T/C)(C/A)GITA(T/C)GTIGC (SEQ
ID NO: 48), and
P4, AAIA(G/A)I(G/C)(A/T)IACIA(T/C)I(G/C)(A/T)IA
(G/A)(A/G)TGI(G/C)(A/T)I(C/G)C (SEQ ID NO: 49).

In the preceding formulae, each parenthesis encloses amino acids which are alternatives to one other, and each slash within such parentheses separates such alternative amino acids.

These DNAs (or a DNA encompassing transmembrane domains 2 through 7 for clone F6) were labeled and tested for crosshybridization at 70° C. Those DNAs which did not show appreciable crosshybridization were hybridized individually, or as a pool to Southern blots at 70° C.

Rat Sequences Used to Obtain Similar Sequences Expressed in Humans

There are genes similar to the rat genes discussed above present in humans, these genes may be readily isolated by screening human gene libraries with the cloned rat sequences or by performing PCR experiments on human genomic DNA with primers homologous to the rat sequences. First, PCR experiments were performed with genomic DNA from rat, human, mouse, and several other species. When primers homologous to transmembrane domains 2 and 6 (the A4/B6 primer set used to isolate the original rat sequences) were used, DNA of the appropriate size was amplified from rat, human and mouse DNAs. When these primary PCR reactions were subsequently diluted and subjected to PCR using primers to internal sequences (P1 and P4 primers), smaller DNA species were amplified whose size was that seen when the same primers were used in PCR reactions with the cloned rat cDNAs. Similarly, when the secondary PCR was performed with one outer primer together with one inner primer (i.e. A4/P4 or P1/B6), amplified DNAs were obtained whose sizes were also consistent with the amplification of genes similar in sequence and organization to the cloned rat cDNAs. Second, a mix of segments from 20 of the rat cDNAs (‘Pi1” probe) was used to screen libraries constructed from human genomic DNAs. Hybridization under high or low stringency conditions reveals the presence of a large number of cloned human DNA segments that are homologous to the rat sequences. Finally, RNA from a human olfactory tumor (neuroesthesioma, NCI-H-1011) cell line has been examined for sequences homologous to those cloned in the rat. cDNA prepared from this RNA was subjected to PCR with the A4/B6 primer set and a DNA species of the appropriate size was seen. This DNA was subcloned and partially sequenced and clearly encodes a member of the olfactory protein family identified in the rat.

The inserted sequence in human clones H3/H5 was amplified by PCR with the A4/B6 primers, gel purified, and then labeled with ³²P. The labeled DNA was then hybridized to restriction enzyme human placenta. Multiple hybridizing species were observed with each DNA (see FIG. 32). This observation is consistent with the presence of a family of odorant receptor genes in the human genome.

The sequence of clone H5 is hereby shown in FIG. 19. In addition, the translated protein sequence is shown in FIG. 19.

In other to identify odorant receptors in other species, degenerated primer oligonucleotides homologous to conserved regions within the rat odorant receptor family may be used in PCR reactions with genomic DNA or with cDNA prepared from olfactory tissue RNA from those species.

Results

Cloning the Gene Family

A series of degenerate oligonucleotides were designated which could anneal to conserved regions of members of the superfamily of G-protein coupled seven transmembrane domain receptor genes. Five degenerate oligonucleotides (A1-5; see Experimental Procedures) matching sequences within transmembrane domain 2, and six degenerate oligonucleotides (B1-6) matching transmembrane domain 7 were used in all combinations in PCR reactions to amplify homologous sequences in cDNA prepared from rat olfactory epithelium RNA. The amplification products of each PCR reaction were then analyzed by agarose gel electrophoresis. Multiple bands were observed with each of the primer combinations. The PCR products within the size range expected for this family of receptors (600 to 1300 bp) were subsequently picked and amplified further with the appropriate primer pair in order to isolate individual PCR bands. Sixty-four PCR bands isolated in this fashion revealed only one or a small number of bands upon agarose gel electrophoresis. Representatives of these isolated PCR products are shown in FIG. 2A.

The isolated PCR products were digested with the endonuclease, Hae III or Hinf I, which recognize four base restriction sites and cut DNA at frequent intervals. In most instances, digestion of the PCR product with Hinf I generated a set of fragments whose molecular weights sum to the size of the original DNA (FIG. 2B). These PCR bands are therefore likely to each contain a single DNA species. In some cases, however, restriction digestion yielded a series of fragments whose molecular weights sum to a value greater than that of the original PCR product. The most dramatic example is shown in FIG. 2 where the 710 bp, PCR 13 DNA, is cleaved by Hinf I to yield a very large number of restriction fragments whose sizes sum to a value five- to ten-fold greater than that of the original PCR product. These observations indicated that PCR product 13 consists of a number of different species of DNA, each of which could be amplified with the same pair of primer oligonucleotides. In addition, when PCR experiments similar to those described were performed using cDNA library DNAs as templates, a 710 bp PCR product was obtained with the PCR13 primer pair (A4/B6) with DNA from olfactory cDNA libraries, but not a glioma cDNA library. Moreover, digestion of one of this 710 bp product also revealed the presence of multiple DNA species. In other cases (see PCR product 20, for example), digestion yielded a series of restriction fragments whose molecular weights also sum to a size greater than the starting material. Further analysis, however, revealed that the original PCR product consisted of multiple bands of similar but different sizes.

In order to determine whether the multiple DNA species present in PCR 13 encode members of a family of seven transmembrane domain proteins, PCR 13 DNA was cloned into the plasmid vector Bluescript and five individual clones were subjected to DNA sequence analysis. Each of the five clones exhibited a different DNA sequence, but each encoded a protein which displayed conserved features of the superfamily of seven transmembrane domain receptor proteins. In addition, the proteins encoded by all five clones shared distinctive sequence motifs not found in other superfamily members indicating they were all members of a new family of receptors.

To obtain full-length cDNA clones, cDNA libraries prepared from olfactory epithelium RNA or from RNA of an enriched population of olfactory sensory neurons were screened. The probe used in these initial screens was a mixture of PCR 13 DNA as well as DNA obtained by amplification of rat genomic DNA or DNA from two olfactory cDNA libraries with the same primers used to generate PCR 13 (A4 and B6 primers). Hybridizing plaques were subjected to PCR amplification with the A4/B6 primer set and only those giving a PCR product of the appropriate size (approximately 710 bp) were purified. The frequency of such positive clones in the enriched olfactory neuron cDNA library was approximately five times greater than the frequency in the olfactory epithelium cDNA library. The increased frequency of positive clones observed in the olfactory neuron library is comparable to the enrichment in olfactory neurons generally obtained in the purification procedure.

The original pair of primers used to amplify PCR 13 DNA were then used to amplify coding segments of 20 different cDNA clones. A mix of these PCR products were labeled and used as probe for further cDNA library screens. This mixed probe was also used in a Northern blot (FIG. 3) to determine whether the expression of the gene family is restricted to the olfactory epithelium. The mixed probe detects two diffuse bands centered at 2 and 5 kb in RNA from olfactory epithelium; no hybridization can be detected in brain or spleen. (Later experiments which examined a larger number of tissue RNAs with a more restricted probe will be shown below.) Taken together, these data indicate the discovery of a novel multigene family encoding seven transmembrane domain proteins which are expressed in olfactory epithelium, and could be expressed predominantly or exclusively in olfactory neurons.

The Protein Sequences of Numerous, Olfactory-specific Members of the Seven Transmembrane Domain Superfamily

Numerous clones were obtained upon screening cDNA libraries constructed from olfactory epithelium and olfactory neuron RNA at high stringency. Partial DNA sequences were obtained from 36 clones; 18 of these cDNA clones are different, but all of them encode proteins which exhibit shared sequence motifs indicating that they are members of the family identified in PCR 13 DNA. A complete nucleotide sequence was determined for coding regions of ten of the most divergent clones (FIG. 4). The deduced protein sequences of these cDNAs defines a new multigene family which shares sequence and structural properties with the superfamily of neurotransmitter and hormone receptors that traverse the membrane seven times. This novel family, however, exhibits features different from any other member of the receptor superfamily thus far identified.

Each of the ten sequences contains seven hydrophobic stretches (19-26 amino acids) that represent potential transmembrane domains. These domains constitute the regions of maximal sequence similarity to other members of the seven transmembrane domain superfamily (see legend to FIG. 4). On the basis of structural homologies with rhodopsin and the β-adrenergic receptors, (19) it is likely that the amino termini of the olfactory proteins are located on the extracellular side of the plasma membrane and the carboxyl termini are located in the cytoplasm. In this scheme, three extracellular loops alternate with three intracellular loops to link the seven transmembrane domains (see FIG. 5). Analysis of the sequences in FIG. 4 demonstrates that the olfactory proteins, like other members of the receptor superfamily, display no evidence of an N-terminal signal sequence. As in several other superfamily members, a potential N-linked glycosylation site is present in all ten proteins within the short N-terminal extracellular segment. Other structural features conserved with previously identified members of the superfamily included cysteine residues at fixed positions within the first and second extracellular loops that are thought to form a disulfide bond. Finally, many of the olfactory proteins reveal a conserved cysteine within the C-terminal domain which may serve as a palmitoylation site anchoring this domain to the membrane (21). These features, taken together with several short, conserved sequence motifs (see legend to FIG. 4), clearly define this new family as a member of the superfamily of genes encoding the seven transmembrane domain receptors.

There are, however, important differences between the olfactory protein family and the other seven transmembrane domain proteins described previously and these differences may be relevant to proposed function of these proteins in odor recognition. Structure-function experiments involving in vitro mutagenesis suggest that adrenergic ligands interact with this class of receptor molecule by binding within the plane of the membrane (22, 20). Not surprisingly, small receptor families that bind the same class of ligands, such as the adrenergic and muscarinic acetylcholine receptor families exhibit maximum sequence conservation (often over 80%) within the transmembrane domains. In contrast, the family of receptors discussed in this application shows striking divergence within the third, fourth, and fifth transmembrane domains (FIG. 4). The variability in the three central transmembrane domains is highlighted schematically in FIG. 5. The divergence in potential ligand binding domains is consistent with the idea that the family of molecules cloned is capable of associating with a large number of odorant of diverse molecular structure.

Receptors which belong to the superfamily of seven transmembrane domain proteins interact with G-proteins to generate intracellular signals. In vitro mutagenesis experiments indicate that one site of association between receptor and G-protein resides within the third cytoplasmic loop (22, 23). The sequence of this cytoplasmic loop in 18 different clones we have characterized is shown in FIG. 6A. This loop which is often quite long and of variable length in the receptor superfamily is relatively short (only 17 amino acids) and of fixed length in the 18 clones examined. Eleven of the 18 different clones exhibit the sequence motif K/R I V S S I (SEQ ID NO: 50 and SEQ ID NO: 51) (or a close relative) at the N-terminus of this loop. Two of the cDNA clones reveal a different H I T C/W A V (SEQ ID NO: 52 and SEQ ID NO: 53) motif at this site. If this short loop is a site of contact with G-proteins, it is possible that the conserved motifs may reflect sites of interaction with different G-proteins that activate different intracellular signaling systems in response to odors. In addition, the receptors cloned reveal several serine or threonine residues within the third cytoplasmic loop. By analogy with other G-protein coupled receptors, these residues may represent sites of phosphorylation for specific receptor kinases involved in desensitization (24).

Subfamilies within the Multigene Family

FIG. 6A displays the sequences of the fifth transmembrane domain and the adjacent cytoplasmic loop encoded by L8 of the cDNA clones we have analyzed. As a group, the 18 sequences exhibit considerable divergence within this region. The multigene family, however, can be divided into subfamilies such that the members of a given subfamily share significant sequence conservation.

In subfamily B, clones F12 and F13, for example, differ from one another at only four of 44 positions (91% identify), and clearly define a subfamily. Clones F5 and Ill (subfamily D) differ from F12 and F13 at 34-36 positions within this region and clearly define a separate subfamily. Thus, this olfactory-specific multigene family consists of highly divergent subfamilies. If these genes encode odor receptors, it is possible that members of the divergent subfamilies bind odorant of widely differing structural classes. Members of the individual subfamilies could therefore recognize more subtle differences between molecules which belong to the same structural class of molecules structures.

The Size of the Multigene Family

Genomic Southern blotting experiments were preformed and genomic libraries were screened to obtain an estimate of the sizes of the multigene family and the member subfamilies encoding the putative odor receptors. DNAs extending from the 3′ end of transmembrane domain 3 to the middle of transmembrane domain 6 were synthesized by PCR from DNA of seven of the divergent cDNA clones (FIG. 4). In initial experiments, these DNAs were labeled and hybridized to each other to define conditions under which minimal crosshybridization would be observed among the individual clones. At 70° C., the seven DNAs showed no crosshybridization, or crossnybridized only very slightly. The trace levels of crosshybridization observed are not likely to be apparent upon genomic Southern blot analysis where the amounts of DNA are far lower than in the test cross.

Probes derived from these seven DNAs were annealed under stringent conditions, either individually or as a group, to Southern blots of rat liver DNA digested with the restriction endonucleases Eco RI or Hind III (FIG. 7).

Examination of the Southern blots reveals that all but one of the cDNAs detects a relatively large, distinctive array of bands in genomic DNA. Clone I15 (probe 7), for example, detects about 17 bands with each restriction endonuclease, whereas clone F9 (probe 1) detects only about 5-7 bands with each enzyme. A single band is obtained with clone I7 (probe 5). PCR experiments using nested primers (TM2/TM7 primers followed by primers to internal sequences) and genomic DNA as template indicate that the coding regions of the members of this multigene family, like those of many members of the G-protein coupled superfamily, may not be interrupted by introns. This observation, together with the fact that most of the probes only encompasses 400 nucleotides suggests that each band observed in these experiments is likely to represent a different gene. These data suggest that the individual probes chosen are representatives of subfamilies which range in size from a single member to as many as 17 members. A total of about 70 individual bands were detected in this analysis which could represent the presence of at least 70 different genes. Although the DNA probes used in these blots did not crosshybridize appreciably with each other, it is possible that a given gene might hybridize to more than one probe, resulting in an overestimate of gene number. However, it is probable that the total number of bands only reflects a minimal estimate of gene number since it is unlikely that we have isolated representative cDNAs from all of the potential subfamilies and the hybridizations were performed under conditions of very high stringency.

A more accurate estimate of the size of the olfactory-specific gene family was obtained by screening rat genomic libraries. The mix of the seven divergent probes used in Southern blots, or the mix of 20 different probes used in our initial Northern blots (see FIG. 3), were used as hybridization probes under high (65° C.) or lowered (55° C.) stringency conditions in these experiments. Nested PCR (see above) was used to verify that the clones giving a positive signal under low stringency annealing conditions were indeed members of this gene family. It is estimated from these studies that there are between 100 and 200 positive clones per haploid genome. The estimate of the size of the family obtain from screens of genomic libraries again represents a lower limit. Given the size of the multigene family, one might anticipate that many of these genes are linked such that a given genomic clone may contain multiple genes. Thus the data from Southern blotting and screens of genomic libraries indicate that the multigene family identified consists of one to several hundred member genes which can be divided into multiple subfamilies.

It should be noted that the cDNA probes isolated may not be representative of the full complement of subfamilies within the larger family of olfactory proteins. The isolation of cDNAs, for example, relies heavily on PCR with primers from transmembrane domains 2 and 7 and biases our clones for homology within these regions. Thus, estimates of gene number as well as subsequent estimates of RNA abundance should be considered as minimal.

Expression of the Members of this Multigene Family

Additional Northern blot analyses were preformed to demonstrate that expression of the members of this gene family is restricted to the olfactory epithelium. (FIG. 8) Northern blot analysis with a mixed probe consisting of the seven divergent cDNAs used above reveals two diffuse bands about 5 and 2 kb in length in olfactory epithelium RNA. This pattern is the same as that seen previously with the mix of 20 DNAs. No annealing is observed to RNA from the brain or retina or other, nonneural tissues, including lung, liver, spleen, and kidney.

An estimate of the level of expression of this family can be obtained from screens of cDNA libraries. The frequency of positive clones in cDNA libraries made from olfactory epithelium RNA suggests that the abundance of the RNAs in the epithelium is about one in 20,000. The frequency of positive clones is approximately five-fold higher in a cDNA library prepared from RNA from purified olfactory neurons (in which 75% of the cells are olfactory neurons). The increased frequency of positive clones obtained in the olfactory neuron cDNA library is comparable to the enrichment we obtain upon purification of olfactory neurons. These observations suggest that this multigene family is expressed largely, if not solely, in olfactory neurons and may not be expressed in other cell types within the epithelium. If each olfactory neuron contains 10⁵ mRNA molecules, from the frequency of positive clones we predict that each neuron contains only 25-30 transcripts derived from this gene family. Since the family of olfactory proteins consists of a minimum of a hundred genes, a given olfactory neuron could maximally express only a proportion of the many different family members. These values thus suggest that olfactory neurons will exhibit significant diversity at the level of expression of these olfactory proteins.

Identification of Pheromone Receptors in Vomeronasal Organ

The vomeronasal organ (vomeronasal gland) is an accessory olfactory structure that is located near the nasal cavity. Like the olfactory epithelium of the vomeronasal organ contains olfactory sensory neurons. The vomeronasal organ is believed to play an important role in the sensing of pheromones in numerous species. Pheromones are believed to have profound effects on both physiological and behavioral aspects of reproduction. The identification of pheromone receptors would permit the identification of the pheromones themselves. It would also enable one to identify agonists or antagonists that would either mimic the pheromones or block the pheromone receptors from transducing pheromone signals. Such information would be important to the development of species specific pesticides and, conversely, to animal husbandry. The identification of pheromone receptors in human could ultimately lead to the development of contraceptives or to treatments for infertility in humans. It is likely that the identification of pheromone receptors in low mammals such as rodents would lead to the identification of similar receptors in human.

In order to identify potential pheromone receptors, we isolate RNA from the vomeronasal organs of female rats and prepared cDNA from this RNA. The cDNA was subjected to PCR with several different pairs of degenerate oligonucleotide primers that match sequences present in the rat odorant receptor family. The PCR products were subcloned and the nucleotide sequences of the subcloned DNAs were determined. Each of the subcloned DNAs encodes a protein that belongs to the odorant receptor family. The sequences of the following vomeronasal subclones are shown: J1 (SEQ ID NOs: 13 and 14), J2 (SEQ ID NOs: 15 and 16), J4 (SEQ ID NOs: 17 and 18), J7 (SEQ ID NOs: 19 and 20), J8 (SEQ ID NOs: 21 and 22), J11 (SEQ ID NOs: 23 and 24), J14 (SEQ ID NOs: 25 and 26), J15 (SEQ ID NOs: 27 and 28), J16 (SEQ ID NOs: 29 and 30), J17 (SEQ ID NOs: 31 and 32), J19 (SEQ ID NOs: 33 and 34), and J20 (SEQ ID NOs: 35 and 36). In a few cases (J2, J4), the same sequence was amplified with two different primer pairs and the sequence shown is a composite of the two sequences. It is possible that one or more of these molecules, or closely related molecules, serve as pheromone receptors in the rat.

Discussion

The mammalian olfactory system can recognize and discriminate a large number of odorous molecules. Perception in this system, as in other sensory systems, initially involves the recognition of external stimuli by primary sensory neurons. This sensory information is then transmitted to the brain where it is decoded to permit the discrimination of different odors. Elucidation of the logic underlying olfactory perception is likely to require the identification of the specific odorant receptors, the analysis of the extent of receptor diversity and receptor specificity, as well as an understanding of the pattern of receptor expression in the olfactory epithelium.

The odorant receptors are thought to transduce intracellular signals by interacting with G-proteins which activate second messenger systems (12, 13, 14, 15). These proteins are clearly members of the family of G-protein coupled receptors which traverse the membrane seven times (19). The odorant receptors should be expressed specifically in the tissue in which odorant are recognized. The family of olfactory proteins cloned is expressed in the olfactory epithelium. Hybridizing RNA is not detected in brain or retina, or in a host of nonneural tissues. Moreover, expression of this gene family the epithelium may be restricted to olfactory neurons. The family of odorant receptors must be capable of interacting with extremely diverse molecular structures. The genes cloned are members of any extremely large multigene family which exhibit variability in regions thought to be important in ligand binding. The possibility that each member of this large family of seven transmembrane proteins is capable of interacting with only one or a small number of odorant provides a plausible mechanism to accommodate the diversity of odor perception. The properties of the gene family identified suggests that this family is likely to encode a large number of distinct odorant receptors.

Size of the Multigene Family

The size of the receptor repertoire is likely to reflect the range of detectable odors and the degree of structural specificity exhibited by the individual receptors. It has been estimated that humans can identify over 10,000 structurally-distinct odorous ligands. However, this does not necessarily imply that humans possess an equally large repertoire of odorant receptors. For example, binding studies in lower vertebrates suggest that structurally-related odorant may activate the same receptor molecules. In fish which smell amino acids, the binding of alanine to isolated cilia can be competed by other small polar residues (threonine and serine), but not by the basic amino acids, lysine or arginine (11). These data suggest that individual receptors are capable of associating with several structurally-related ligands, albeit with different affinities. Stereochemical models of olfactory recognition in mammals (25) (based largely on psychophysical, rather than biophysical data) have suggested existence of several primary odor groups including camphoraceous, musky, peppermint, ethereal, pungent, and putrid. In such a model, each group would contain odorant with common molecular configurations which bind to common receptors and share similar odor qualities.

Screens of genomic libraries with mixed probes consisting of divergent family members detect approximately 100 to 200 positive clones per genome. The present estimate of at least 100 genes provides only a lower limit since it is likely that the probes used do not detect all of the possible subfamilies. Moreover, it is probable that many of these genes are linked such that a given genomic clone may contain multiple genes. It is therefore expected that the actual size of the gene family may be considerably higher and this family of putative odorant receptors could constitute one of the largest gene families in the genome.

The characterization of a large multigene family encoding putative odorant receptors suggests that the olfactory system utilizes a far greater number of receptors than the visual system. Color vision, for example, allows the discrimination of several hundred hues, but is accomplished by only three different photoreceptors (1, 2, 3 and 4). The photoreceptors each have different, but overlapping absorption spectra which cover the entire spectrum of visible wavelengths. Discrimination of color results from comparative processing of the information from these three classes of photoreceptors in the brain. Whereas three photoreceptors can absorb light across the entire visible spectrum, our data suggest that a small number of odorant receptors cannot recognize and discriminate the full spectrum of distinct molecular structures perceived by the mammalian olfactory system. Rather, olfactory perception probably employs an extremely large number of receptors each capable of recognizing a small number of odorous ligands.

Diversity within the Gene Family and the Specificity of Odor Recognition

The olfactory proteins identified in this application are clearly members of the superfamily of receptors which traverse the membrane seven times. Analysis of the proteins encoded by the 18 distinct cDNAs we have cloned reveals structural features which may render this family particularly well suited for the detection of a diverse array of structurally distinct odorant. Experiments with other members of this class of receptors suggest that the ligand binds to its receptor within the plane of the membrane such that the ligand contacts many, if not all of the transmembrane helices. The family of olfactory proteins can be divided into several different subfamilies which exhibit significant sequence divergence within the transmembrane domains. Nonconservative changes are commonly observed within blocks of residues in transmembrane regions 3, 4, and 5 (FIGS. 4, 5, 6); these blocks could reflect the sites of direct contact with odorous ligands. Some members, for example, have acidic residues in transmembrane domain 3, which in other families are thought to be essential for binding aminergic ligands (20) while other members maintain hydrophobic residues at these positions. This divergence within transmembrane domains may reflect the fact that the members of the family of odorant receptors must associate with odorant of widely different molecular structures.

These observations suggest a model in which each of the individual subfamilies encode receptors which bind distinct structural classes of odorant. Within a given subfamily, however, the sequence differences are far less dramatic and are often restricted to a small number of residues. Thus, the members of a subfamily may recognize more subtle variations among odor molecules of a given structural class. At a practical level, individual subfamilies may recognize grossly different structures such that one subfamily may associate, for example, with the aromatic compound, benzene and its derivatives, whereas a second subfamily may recognize odorous, short chain, aliphatic molecules. Subtle variations in the structure of the receptors within, for example, the hypothetical benzene subfamily could facilitate the recognition and discrimination of various substituted derivatives such as toluene, xylene or phenol. It should be noted that such a model, unlike previous stereochemical models, does not necessarily predict that molecules with similar structures will have similar odors. The activation of distinct receptors with similar structures could elicit different odors, since perceived odor will depend upon higher order processing of primary sensory information.

Identification of Odorant Ligands for Members of the Gene Family

Odorant ligands have been identified for members of the gene family disclosed herein. For the I7 odorant receptor, these include octanal (44, 47), heptanal (45), trans, trans-2,4,-octadienal, tetrahydrocitral, and citronella (44). In addition, a variety of different aliphatic odorants have been described for 14 different odorant receptors (46). One odorant receptor can recognize multiple odorants and one odorant is recognized by multiple odorant receptors, but different odorants are recognized by different combinations of odorant receptors (46).

Evolution of the Gene Family and the Generation of Diversity

Preliminary evidence from PCR analyses suggests that members of this family of olfactory proteins are conserved in lower vertebrates as well as invertebrates. This gene family presumably expanded over evolutionary time providing mammals with the ability to recognize an increasing diversity of odorant. Examination of the sequences of the family members cloned from mammals provides some insight into the evolution of this multigene family. Although the chromosomal loci encoding these genes has yet to be characterized, it is likely that at least some member genes will be tandemly arranged in a large cluster as is observed with other large multigene families. A tandem array of this sort provides a template for recombination events including unequal crossing over and gene conversion, that can lead to expansion and further diversification of the sort apparent among the family members we have cloned (26).

The multigene family encoding the olfactory proteins is large: all of the member genes clearly have a common ancestral origin, but have undergone considerable divergence such that individual genes encode proteins that share from 40-80% amino acid identity. Subfamilies are apparent with groups of genes sharing greater homology among themselves than with members of other subfamilies. Examination of the sequences of even the most divergent subfamilies, however, reveals a pattern in which several blocks of conserved residues are interspersed with variable regions. This segmental homology is conceptually similar to the organization of framework and hypervariable domains within the families of immunoglobulin and T cell receptor variable region sequences (27, 28). This analogy goes beyond structural organization and may extend to the function of these two gene families: each family consists of a large number of genes which have diversified over evolutionary time to accommodate the binding of a highly diverse array of ligands. The evolutionary mechanisms responsible for the diversification and maintenance of these large gene families may also be similar. It has been suggested that gene conversion has played a major role in the evolution of immunoglobulin and T cell receptor variable domains (29, 30 and 31). Analysis of the sequence of the putative olfactory receptors reveals at least one instance where a motif from a variable region of one subfamily is found imbedded in the otherwise divergent sequence of a second subfamily, suggesting that conversion has occurred. Such a mixing of motifs from one subfamily to another over evolutionary time would provide additional combinatorial possibilities leading to the generation of diversity.

It should be noted, however, that the combinatorial joining of gene segments by DNA rearrangement during development, which is characteristic of immunoglobulin loci (27), is not a feature of the putative odor receptor gene family. No evidence for DNA rearrangement to generate the diversity of genes cloned has been observed. The entire coding region has been sequenced along with parts of the 5′ and 3′ untranslated regions of 10 different cDNA clones. The sequences of the coding regions are all different; no evidence has been obtained for constant regions that would suggest DNA rearrangement of the sort seen in the immune system. The observations indicate that the diversity olfactory proteins are coded by a large number of distinct gene sequences.

Although it is unlikely from the data that DNA rearrangement is responsible for the generation of diversity among the putative odorant receptors, it remains possible that DNA rearrangements may be involved in the regulation of expression of this gene family. If each olfactory neuron expresses only one or a small number of genes, then a transcriptional control mechanism must be operative to choose which of the more than one hundred genes within the family will be expressed in a given neuron. Gene conversion from one of multiple silent loci into a single active locus, as observed for the trypanosome-variable surface glycoproteins (32), provides one attractive model. The gene conversion event could be stochastic, such that a given neuron could randomly express any one of several hundred receptor genes, or regulated (perhaps by positional information) such that a given neuron could only express one or a small number of predetermined receptor types. Alternatively, it is possible that positional information in the olfactory epithelium controls the expression of the family of olfactory receptors by more classical mechanisms that do not involve DNA rearrangement. What ever mechanisms will regulate the expression of receptor genes within this large, multigene family, these mechanisms must accommodate the requirement that olfactory neurons are regenerated every 30-60 days (8) and therefore the expression of the entire repertoire of receptors must be accomplished many times during the life of an organism.

Receptor Diversity and the Central Processing of Olfactory Information

The results suggest the existence of a large family of distinct odorant receptors. Individual members of this receptor family are likely to be expressed by only a small set of the total number of olfactory neurons. The primary sensory neurons within the olfactory epithelium will therefore exhibit significant diversity at the level of receptor expression. The question then emerges as to whether neurons expressing the same receptors are localized in the olfactory epithelium. Does the olfactory system employ a topographic map to discriminate among the numerous odorant? The spatial organization of distinct classes of olfactory sensory neurons, as defined by receptor expression, can now be determined by using the procedures of in situ hybridization and immunohistochemistry with probes specific for the individual receptor subtypes. This information should help to distinguish between different models that have been proposed to explain the coding of diverse odorant stimuli (33).

In one model, sensory neurons that express a given receptor and respond to a given odorant may be localized within defined positions within the olfactory epithelium. This topographic arrangement would also be reflected in the projection of olfactory sensory axons into discrete regions (glomeruli) within the olfactory bulb. In this scheme, the central coding to permit the discrimination of discrete odorant would depend, in part, on the spatial segregation of different receptor populations. Attempts to discern the topographic localization of specific receptors at the level of the olfactory epithelium has led to conflicting results. In some studies, electrophysiological recordings have revealed differences in olfactory responses to distinct odorant in different regions of the olfactory epithelium (34, 35). However, these experiments have been difficult to interpret since the differences in response across the epithelium are often small and are not observed in all studies (36).

A second model argues that sensory neurons expressing distinct odorant receptors are randomly distributed in the epithelium but that neurons responsive to a given odorant project to restricted regions within the olfactory bulb. In this instance, the discrimination of odors would be a consequence of the position of second order neurons in the olfactory bulb, but would be independent of the site of origin of the afferent signals within the epithelium. Mapping of the topographic projections of olfactory neurons has been performed by extracellular recordings from different regions of the bulb (37, 38) and by 2-deoxyglucose autoradiography to map regional activity after exposure to different odorant (39). These studies suggest that spatially-localized groups of bulbar neurons preferentially respond to different odorant. The existence of specific odorant receptors, randomly distributed through the olfactory epithelium, which converge on a common target within the olfactory bulb, would raise additional questions about the recognition mechanisms used to guide these distinct axonal subsets to their central targets.

Other sensory systems also spatially segregate afferent input from primary sensory neurons. The spatial segregation of information employed, for example, by the visual and somatosensory systems, is used to define the location of the stimulus within the external environment as well as to indicate the quality of the stimulus. In contrast, olfactory processing does not extract spatial features of the odorant stimulus. Relieved of the necessity to encode information about the spatial localization of the sensory stimulus, it is possible that the olfactory system of mammals uses the spatial segregation of sensory input solely to encode the identity of the stimulus itself. The molecular identification of the genes likely to encode a large family of olfactory receptors should provide initial insights into the underlying logic of olfactory processing in the mammalian nervous system.

### REFERENCES

1. Rushton, W. A. H. (1965) A Foveal pigment in the deuteranope. J. Physiol. 176, 24-37.

2. Rushton, W. A. H. (1955) Foveal photopigments in normal and colour-blind. J. Physiol. 129, 41-42.

3. Wald, G., Brown, P. K., and Smith, P. H. (1955) Iodopsin. J. Gen. Physiol. 38, 623-681.

4. Nathans, J., Thomas, D., and Hogness, D. S. (1986) Molecular genetics of human color vision: The genes encoding blue, green and red pigments. Science 232, 193-202.

5. Lancet, D. (1986) Vertebrate olfactory reception. Ann. Rev. Neurosci. 9, 329-355.

6. Reed, R.R. (1990) How does the nose know? Minireview. Cell 60, 1-2.

7. Moulton, D. G., and Beidler, L. M. (1967) Structure and function in the peripheral olfactory system. Physiol. Rev. 47, 1-52.

8. Graziadei, P. P. C., and Monti Graziadei, G. A. (1979) Neurogenesis and neuron regeneration in the olfactory system of mammals. I. Morphological aspects of differentiation and structural organization of the olfactory sensory neurons. J. Neurocytol. 8, 1-18.

9. Bronshtein, A. A., and Minor, A. V. (1977) Regeneration of olfactory flagella and restoration of the electroolfactogram following application of triton X-100 to the olfactory mucosa of frogs. Tsitologiia 19, 33-39.

10. Rhein, L. D., and Cagan, R. H. (1980) Biochemical studies of olfaction: Isolation, characterization, and odorant binding activity of cilia from rainbow trout olfactory rosettes. Proc. Natl. Acad. Sci. USA 77, 4412-4416.

11. Rhein, L. D., and Cagan, R. H. (1983) Biochemical studies of olfaction: Binding specificity of odorant to a cilia preparation from rainbow trout olfactory rosettes. J. Neurochem. 41, 569-577.

12. Pace, U., Hanski, E., Salomon, Y., and Lancet, D. (1985) Odorant-sensitive adenylate cyclase may mediate olfactory reception. Nature 316, 255-258.

13. Sklar, P. B., Anholt, R. R. H.; and Snyder, S. H. (1986) The odorant-sensitive adenylate cyclase of olfactory receptor cells: Differential stimulation by distinct classes of odorant. J. Biol. Chem. 261, 15538-15543.

14. Breer, H., Boekhoff, I., and Tareilus, E. (1990) Rapid kinetics of second messenger formation in olfactory transduction. Nature 345, 65-68.

15. Boekhoff, I., Tareilus, E., Strotmann, J., and Breer, H. (1990). Rapid activation of alternative second messenger pathways in olfactory cilia from rats by different odorant. EMBO J. 9, 2453

16. Jones, D. T., and Reed, R. R. (1989) G_{olf}: an olfactory neuron specific-G-protein involved in odorant signal transduction. Science 244, 790-795.

17. Nakamura, T. and Gold G. (1987) A cyclic nucleotide-gated conductance in olfactory receptor cilia. Nature 325, 442-444.

18. Dhallan, R. S., Yau, K. -W., Schrader, K. A., and Reed, R. R. (1990) Primary structure and functional expression of a cyclic nucleotide-activated channel from olfactory neurons. Nature 347, 184-187.

19. O'Dowd, B. F., Felkowitz, R. J., and Caron, M. G. (1989) Structure of the adrenergic and related receptors. Ann. Rev. Neurosci. 12, 67-83.

20. Strader, C. D., Sigal, I. S., and Dixon, R. F. (1989) Structural basis of adrenergic receptor function FASEB J. 3, 1825-1832.

21. O'Down, B. F., Hnatowich, M., Caron, M. G., Lefkowitz, R. J., and Bouvier, M. (1989) Palmitoylation of the human β₂-adrenergic receptor. Mutation of CYS³⁴¹ in the carboxyl tail leads to an uncoupled nonpalymitoylated form of the receptor. J. Biol. Chem. 264, 7564-7569.

22. Kobilka, B. KI., Kobilka, T. S., Daniel, K., Regan, J. W., Caron, M. G., and Lefkowitz, R. J. (1988) Chimeric α₂-β₂-ldrenergic receptors: delineation of domains involved in effector coupling and ligand binding specificity. Science 240, 1310-1316.

23. Hamm, H. E., Deretic, D., Arendt, A., Hargrove, P. A., Koenig, B., and Hofmann, K. P. (1988) Site of G protein binding to rhodopsin mapping with synthetic peptides to the a subunit. Science 241, 832-835. (1988).

24. Bouvier, M. W., Hausdorff, A., De Blasi, A., O'Dowd, B. F., Kobilka, B. K., Caron, M. G., and Lefkowitz, R. J. (1988) Removal of phosphorylation sites from the β-adrenergic receptor delays the onset of agonist-promoted desensitization. Nature (London) 333, 370-373.

25. Amoore, J. E. (1982) Odor Theory and Odor Classification, In Fragrance Chemistry (New York: Academic Press, Inc.), pp. 27-73.

26. Maeda, N., and Smithies, 0. (1986) The evolution of multigene families: Human haptoglobin genes. Ann. Rev. Genet. 20, 81-108.

27. Tonegawa, S. (1983) Somatic generation of antibody diversity. Nature 302, 575-581.

28. Hood, L., Kronenberg, M., and Hunkapiller, T. (1985) T cell antigen receptors and the immunoglobulin supergene family. Cell 40, 225-229.

29. Baltimore, D. (1981) Gene conversion: Some implications for immunoglobulin genes. Minireview. Cell 24, 592-594.

30. Egel, R. (1981) Intergenic conversion and reiterated genes. Nature 290, 191-192.

31. Flanagan, J. G., Lefranc, M. -P., and Rabbitts, T. H. (1984) Mechanisms of divergence and convergence of the human immunoglobulin α1 and α2 constant region gene sequences. Cell 36, 681-688.

32. Van der Ploeg, L. H. T. (1991) Antigenic variation in African trypanosomes: genetic recombination and transcriptional control of VSG genes. In Gene Rearrangement. (New York: Oxford University Press), pp 51-98.

33. Shepherd, G. M. (1985) Are there labeled lines in the olfactory pathway? In Taste, Olfaction and the Central Nervous System (New York: The Rockefeller University Press), pp. 307-321.

34. Mackay-Sim, A., Shaman, P., and Moulton, D. G. (1982) Topographic coding of olfactory quality: odorant-specific patterns of epithelial responsivity in the Salamander. J. Neurophysiol. 48, 584-596.

35. Thommesen, G., and Doving, K. B. (1977) Spatial distribution of the EOG in the rat. A variation with odour stimulation. Acta Physiol. Scand. 99, 270-280.

36. Sicard, G. (1985) Olfactory discrimination of structurally related molecules: Receptor cell responses to camphoraceous odorant. Brain Res. 326, 203-212.

37. Thommesen, G. (1978) The spatial distribution of odour induced potentials in the olfactory bulb or char and trout (Salmonidae). Acta Physiol. Scand. 102, 205-217.

38. Doving, K. B., Selset, R., and Thommesen, G. (1980) Olfactory sensitivity to bile acids in salmonid fishes. Acta Physiol. Scand. 108, 123-131.

39. Stewart, W. B., Kauer, J. S., and Shepherd, G. M. (1979) Functional organization of the rat olfactory bulb analyzed by the 2-deoxyglucose method. J. Comp. Neurol. 185, 715-734.

40. Chirgwin, J. M., Przbyla, A. E., MacDonald, R. J., and Rutter, W. J. (1979) Isolation of biologically active ribonucleic acid from sources enriched in ribonuclease. Biochemistry 18, 5294-5299.

41. Maniatis, T., Fritsch, E., and Sambrook, J. (1982) Molecular Cloning: A Laboratory Manual (Cold Spring Harbor, New York: Cold Spring Harbor Laboratory Press).

42. Sambrook, J., Fritsch, E. F., and Maniatis, T. (1989) Molecular Cloning: A Laboratory Manual, Second Edition (Cold Spring Harbor, New York, Cold Spring Harbor Laboratory Press).

43. Julius et al. (1988) Molecular Charatization of a Functional cDNA Encoding a Seritonin 1c Receptor. Science 241, 558-564.

44. Araneda R. C., Kini A. D., and Firestein S. (2000) The molecular receptive range of an odorant receptor. Nature Neuroscience 3(12), 1248-1255.

45. Krautwurst D., Yau K. -W., and Reed R. R. (1998) Identification of ligands for olfactory receptors by functional expression of a receptor library. Cell 95, 917-926.

46. Malnic B., Hirono J., Sato T., and Buck, L. B. (1999) Combinatorial receptor codes for odors. Cell 96, 713-723.

47. Zhao H., Ivic L., Otaki J. M., Hashimoto M., Mikoshiba K., and Firestein S. (1998) Functional expression of a mammalian odorant receptor. Science 279, 237-242.

What is claimed is:

1. An isolated nucleic acid molecule encoding an odorant receptor protein, wherein the receptor protein comprises seven transmembrane domains, and is further characterized by at least one of the following characteristics:
(a) the loop between the first transmembrane domain and the second transmembrane domain, and the second transmembrane domain together comprise consecutive amino acids having the following sequence:
-L, X, X, P, M, Y, X, F, L- (SEQ ID NO: 55);
(b) the third transmembrane domain, and the loop between the third transmembrane domain and the fourth transmembrane domain together comprise consecutive amino acids having one of the following sequences:
-M, X, Y, D, R, X, X, A, I, C- (SEQ ID NO: 57); or
-D, R, X, X, A, I, C- (SEQ ID NO: 59);
(c) the loop between the fifth transmembrane domain and the sixth transmembrane domain, and the sixth transmembrane domain together comprise consecutive amino acids having one of the following sequences:
-K or R, X, F, S, T, C, X, S, H- (SEQ ID NO: 61); or
-F, S, T, C, X, S, H- (SEQ ID NO: 63); or
(d) the seventh transmembrane domain and the C-terminal domain together comprise consecutive amino acids having one of the following sequences:
-P, X, X, N, P, X, I, Y, X, L, R, N- (SEQ ID NO: 65); or
-P, X, X, N, P, X, I, Y- (SEQ ID NO: 67); or
-N, P, X, I, Y, X, L, R, N- (SEQ ID NO: 69);
wherein X is any amino acid.

2. The isolated nucleic acid molecule of claim 1 wherein:
(a) the loop between the first transmembrane domain and the second transmembrane domain, and the second transmembrane domain together comprise consecutive amino acids having the following sequence:
-L, H or Q, K or M or T, PMY, F or L, FL- (SEQ ID NO: 56);
(b) the third transmembrane domain, and the loop between the third transmembrane domain and the fourth transmembrane domain together comprise consecutive amino acids having one of the following sequences:
-M, A or S, YDR, F or Y, L or V, AIC- (SEQ ID NO: 58); or
-DR, F or Y, L or V, AIC- (SEQ ID NO: 60);
(c) the loop between the fifth transmembrane domain and the sixth transmembrane domain, and the sixth transmembrane domain together comprise consecutive amino acids having one of the following sequences:
-K or R, A or I or S or V, FSTC, A or G or S, SH- (SEQ ID NO: 62); or
-FSTC, A or G or S, SH- (SEQ ID NO: 64); or
(d) the seventh transmembrane domain and the C-terminal domain together comprise consecutive amino acids having one of the following sequences:
-P, M or L or V, F or L or V, NP, F or I, IY, C or S or T, LRN- (SEQ ID NO: 66); or
-P, M or L or V, F or L or V, NP, F or I, IY- (SEQ ID NO: 68); or
-NP, F or I, IY, C or S or T, LRN- (SEQ ID NO: 70).

3. The isolated nucleic acid molecule of claim 1, wherein the receptor protein is characterized by at least two of the characteristics (a) through (d).

4. The isolated nucleic acid molecule of claim 1, wherein the receptor protein is characterized by at least three of the characteristics (a) through (d).

5. The isolated nucleic acid molecule of claim 1, wherein the receptor protein is characterized by all of the characteristics (a) through (d).

6. An isolated nucleic acid molecule encoding an odorant receptor protein, wherein the nucleic acid molecule encodes a protein selected from the group consisting of:
(a) an odorant receptor protein comprising consecutive amino acids having a sequence identical to that beginning with methionine at position 1 and ending with tyrosine at position 333 as set forth in row F3 of FIGS. 4A to 4M (SEQ ID NO: 71),
(b) an odorant receptor protein comprising consecutive amino acids having a sequence identical to that beginning with methionine at position 1 and ending with glutamine at position 313 as set forth in row F5 of FIGS. 4A to 4L (SEQ ID NO: 72),
(c) an odorant receptor protein comprising consecutive amino acids having a sequence identical to that beginning with methionine at position 1 and ending with lysine at position 311 as set forth in row F6 of FIGS. 4A to 4L (SEQ ID NO: 73),
(d) an odorant receptor protein comprising consecutive amino acids having a sequence identical to that beginning with methionine at position 1 and ending with glycine at position 317 as set forth in row F12 of FIGS. 4A to 4L (SEQ ID NO: 74),
(e) an odorant receptor protein comprising consecutive amino acids having a sequence identical to that beginning with methionine at position 1 and ending with leucine at position 310 as set forth in row I3 of FIGS. 4A to 4L (SEQ ID NO: 75),
(f) an odorant receptor protein comprising consecutive amino acids having a sequence identical to that beginning with methionine at position 1 and ending with glycine at position 327 as set forth in row I7 of FIGS. 4A to 4L (SEQ ID NO: 76),
(g) an odorant receptor protein comprising consecutive amino acids having a sequence identical to that beginning with methionine at position 1 and ending with tryptophan at position 312 as set forth in row I8 of FIGS. 4A to 4L (SEQ ID NO: 77),
(h) an odorant receptor protein comprising consecutive amino acids having a sequence identical to that beginning with methionine at position 1 and ending with leucine at position 314 as set forth in row I9 of FIGS. 4A to 4L (SEQ ID NO: 78),
(i) an odorant receptor protein comprising consecutive amino acids having a sequence identical to that beginning with methionine at position 1 and ending with leucine at position 312 as set forth in row I14 of FIGS. 4A to 4L (SEQ ID NO: 79),
(j) an odorant receptor protein comprising consecutive amino acids having a sequence identical to that beginning with methionine at position 1 and ending with leucine at position 314 as set forth in row I15 of FIGS. 4A to 4L (SEQ ID NO: 80), and
(k) an odorant receptor protein that shares from 40-80% amino acid identity with any one of the proteins of (a)-(j), comprises seven transmembrane domains, and is further characterized by at least one of the following characteristics:
(i) the loop between the first transmembrane domain and the second transmembrane domain, and the second transmembrane domain together comprise consecutive amino acids having the following sequence: -L, X, X, P, M, Y, X, F, L- (SEQ ID NO: 55);
(ii) the third transmembrane domain, and the loop between the third transmembrane domain and the fourth transmembrane domain together comprise consecutive amino acids having one of the following sequences:
-M, X, Y, D, R, X, X, A, I, C- (SEQ ID NO: 57); or
-D, R, X, X, A, I, C- (SEQ ID NO: 59);
(iii) the loop between the fifth transmembrane domain and the sixth transmembrane domain, and the sixth transmembrane domain together comprise consecutive amino acids having one of the following sequences:
-K or R, X, F, S, T, C, X, S, H- (SEQ ID NO: 61); or
-F, S, T, C, X, S, H- (SEQ ID NO: 63); or
(iv) the seventh transmembrane domain and the C-terminal domain together comprise consecutive amino acids having one of the following sequences:
-P, X, X, N, P, X, I, Y, X, L, R, N- (SEQ ID NO: 65); or
-P, X, X, N, P, X, I, Y- (SEQ ID NO: 67); or
-N, P, X, I, Y, X, L, R, N- (SEQ ID NO: 69);
wherein X is any amino acid.

7. The isolated nucleic acid molecule of claim 6 wherein:
(i) the loop between the first transmembrane domain and the second transmembrane domain, and the second transmembrane domain together comprise consecutive amino acids having the following sequence:
-L, H or Q, K or M or T, PMY, F or L, FL- (SEQ ID NO: 56);
(ii) the third transmembrane domain, and the loop between the third transmembrane domain and the fourth transmembrane domain together comprise consecutive amino acids having one of the following sequences:
-M, A or S, YDR, F or Y, L or V, AIC- (SEQ ID NO: 58); or
-DR, F or Y, L or V, AIC- (SEQ ID NO: 60);
(iii) the loop between the fifth transmembrane domain and the sixth transmembrane domain, and the sixth transmembrane domain together comprise consecutive amino acids having one of the following sequences:
-K or R, A or I or S or V, FSTC, A or G or S, SH- (SEQ ID NO: 62); or
-FSTC, A or G or S, SH- (SEQ ID NO: 64); or
(iv) the seventh transmembrane domain and the C-terminal domain together comprise consecutive amino acids having one of the following sequences:
-P, M or L or V, F or L or V, NP, F or I, IY, C or S or T, LRN- (SEQ ID NO: 66); or
-P, M or L or V, F or L or V, NP, F or I, IY- (SEQ ID NO: 68); or
-NP, F or I, IY, C or S or T, LRN- (SEQ ID NO: 70).

8. An isolated nucleic acid molecule encoding an odorant receptor protein, wherein the nucleic acid molecule comprises a nucleic acid sequence which can be amplified by polymerase chain reaction using:
(a) any one of 5′ primers Al (SEQ ID NO: 37), A2 (SEQ ID NO: 38), A3 (SEQ ID NO: 39), A4 (SEQ ID NO: 40), or A5 (SEQ ID NO: 41); and
(b) any one of 3′ primers E1 (SEQ ID NO: 42), B2 (SEQ ID NO: 43), B3 (SEQ ID NO: 44), B4 (SEQ ID NO: 45), B5 (SEQ ID NO: 46), or B6 (SEQ ID NO: 47).

9. An isolated nucleic acid molecule encoding an odorant receptor protein, wherein the nucleic acid molecule comprises:
(a) a nucleic acid sequence given in any one of FIGS. 9 to 31 (SEQ ID Nos: 1-10, 11, 13, 15, 17, 19, 21, 23, 25, 27, 29, 31, 33, or 35); or
(b) a nucleic acid sequence degenerate to a sequence of (a) as a result of the genetic code.

10. The isolated nucleic acid molecule of claim 8 or 9, wherein the odorant receptor protein comprises seven transmembrane domains.

11. The isolated nucleic acid molecule of claim 1, wherein the loop between the fifth and sixth transmembrane domains consists of 17 amino acids.

12. The isolated nucleic acid molecule of claim 10, wherein the loop between the fifth and sixth transmembrane domains consists of 17 amino acids.

13. The isolated nucleic acid molecule of any one of claims 1, 6, 8, or 9, wherein the odorant receptor is a vertebrate odorant receptor.

14. The isolated nucleic acid molecule of claim 13, wherein the vertebrate odorant receptor is a fish odorant receptor or a mammalian odorant receptor.

15. The isolated nucleic acid molecule of claim 14, wherein the mammalian odorant receptor is a human odorant receptor, a rat odorant receptor, a mouse odorant receptor or a dog odorant receptor.

16. The isolated nucleic acid molecule of claim 1, 6, 8, or 9, wherein the nucleic acid is DNA.

17. The isolated nucleic acid molecule of claim 16, wherein the DNA is cDNA.

18. A vector comprising the isolated nucleic acid molecule of claim 1, 6, 8, or 9.

19. The vector of claim 18, wherein the vector additionally comprises elements necessary for replication and expression in a suitable host.

20. A purified odorant receptor protein encoded by the isolated nucleic acid molecule of claim 1, 6, 8, or 9.

21. A cell transfected with the vector of claim 19.

22. The cell of claim 21, wherein the cell is an olfactory cell.

23. The cell of claim 21, wherein the cell is a non-olfactory cell.

24. The cell of claim 23, wherein prior to being transfected with the vector the non-olfactory cell does not express an odorant receptor protein.

25. A method of identifying a desired odorant ligand, which comprises contacting a non-olfactory cell of claim 23, which express on its cell surface a known odorant receptor, with a series of odorant ligands and determining which ligands bind to the known odorant receptor on the non-olfactory cell.

26. A method of identifying a desired odorant receptor, which comprises contacting a series of non-olfactory cells of claim 23 with a known odorant ligand and determining which odorant receptor binds with the odorant ligand.

27. A method of detecting an odor which comprises:
(a) identifying an odorant receptor which binds the desired odorant ligand identified by the method of claim 26; and
(b) imbedding the receptor in a membrane such that when the odorant ligand binds with the receptor identified in (a) above, a detectable signal is produced.

28. The method of claim 27 wherein the desired odorant ligand is a pheromone.

29. The method of claim 27 wherein the desired odorant ligand is the vapor emanating from cocaine, marijuana, heroin, hashish, angel dust, gasoline, natural gas, alcohol, decayed human flesh, gun powder, an explosive, a plastic explosive, or a firearm.

30. The method of claim 27 wherein the desired odorant ligand is a toxic fume, a noxious fume or a dangerous fume.

31. The method of claim 27 wherein the membrane is a cell membrane, an olfactory cell membrane, or a synthetic membrane.

32. The method of claim 27 wherein the detectable signal is a color change, a phosphorescence, a radioactivity, a visual signal, or an auditory signal.

33. A method of quantifying the amount of an odorant ligand present in a sample which comprises the method of claim 27 wherein the detectable signal is quantified.

34. A method of developing fragrances, which comprises identifying a desired odorant receptor by the method of claim 26, then contacting a non-olfactory cell, which has been transfected with an expression vector comprising an isolated nucleic acid molecule encoding the desired odorant receptor such that the receptor is expressed upon the surface of the non-olfactory cell, with a series of compounds to determine which compounds bind with the receptor.

35. A method of identifying an odorant fingerprint, which comprises contacting a series of cells, which have been transformed such that each express a known odorant receptor encoded by a nucleic acid molecule of any one of claims 1, 6, 8, or 9, with a desired sample containing one or more odorant ligand and determining the type and quantity of the odorant ligands present in the sample.

36. A method of identifying a compound which inhibits an odorant receptor, which comprises contacting an odorant receptor encoded by the nucleic acid molecule of any one of claims 1, 6, 8, or 9 with a series of compounds and determining which compound inhibits interaction between the odorant receptor and an odorant ligand known to bind to the odorant receptor.

37. A method for identifying an appetite suppressant compound, which comprises identifying a compound by the method of claim 36 wherein the odorant receptor is associated with the perception of food.

38. A pharmaceutical composition comprising a compound identified by the method of claim 37 and a pharmaceutically acceptable carrier.

39. A nasal spray for controlling appetite, which comprises a compound identified by the method of claim 37 in a suitable carrier.

40. A method for controlling appetite in a subject, which comprises administering to the subject an amount of a compound identified by the method of claim 37 effective to control the subject's appetite.

41. The method of claim 40, which comprises administering the compound to the subject's olfactory epithelium.

42. A method of trapping odors, which comprises contacting a membrane comprising a plurality of a desired odorant receptor encoded by the nucleic acid molecule of any one of claims 1, 6, 8, or 9 with a sample comprising a desired odorant ligand such that the desired odorant ligand is absorbed by the binding of the odorant ligand to the odorant receptor.

43. An odor trap, which comprises a membrane comprising a plurality of a desired odorant receptor encoded by the nucleic acid molecule of any one of claims 1, 6, 8, or 9, such that a desired odorant ligand is absorbed by the binding of the odorant ligand to the odorant receptor.

44. A method for controlling a pest population in an area, which comprises spraying the area with an odorant receptor ligand identified by the method of claim 25.

45. The method of claim 44, wherein the odorant ligand is an alarm odorant ligand.

46. The method of claim 44, wherein the odorant ligand interferes with an interaction between an odorant ligand and an odorant receptor associated with fertility.

47. The method of claim 44, wherein the pest population is a population of rodents, mice, or rats.

48. A pharmaceutical composition comprising an odorant ligand identified by the method of claim 25 and a pharmaceutically acceptable carrier.

49. A method of promoting fertility in a subject which comprises administering to the subject an amount of an odorant ligand identified by the method of claim 25 effective to promote the subject's fertility.

50. The method of claim 49, wherein the odorant ligand interacts with an odorant receptor associated with fertility.

51. A method of inhibiting fertility in a subject which comprises administering to the subject an amount of an odorant ligand identified by the method of claim 25 effective to inhibit the subject's fertility.

52. The method of claim 51, wherein the odorant ligand inhibits an interaction between an odorant ligand and an odorant receptor associated with fertility.

53. The method of claim 49 or 51, which comprises administering the odorant ligand to the subject's olfactory epithelium.

54. Use of an odorant ligand identified by the method of claim 25 for the preparation of a pharmaceutical composition for controlling a pest population in a desired area by spraying the desired area with the identified odorant ligand.

55. The use of claim 54, wherein the odorant ligand is an alarm odorant ligand.

56. Use of an odorant ligand identified by the method of claim 25 for the preparation of a pharmaceutical composition for controlling a pest population.

57. The use of claim 56, wherein the odorant ligand interferes with the interaction between odorant ligands and odorant receptors which are associated with fertility.

58. Use of any one of claims 54 to 57 wherein the pest population is a population of rodents, mice, or rats.

59. Use of an odorant ligand identified by the method of claim 25 for the preparation of a pharmaceutical composition for promoting fertility.

60. The use of claim 59, wherein the odorant ligand interacts with odorant receptors associated with fertility.

61. Use of an odorant ligand identified by the method of claim 25 for the preparation of a pharmaceutical composition for inhibiting fertility.

62. The use of claim 61, wherein the odorant ligand inhibits the interaction between odorant ligands and odorant receptors associated with fertility.

63. Use of the compound identified by the method of claim 37 for the preparation of a pharmaceutical composition for controlling appetite in a subject.
